SEQUENCE LISTING (2 sequences)

SEQ ID NO: 1 - protein, 17 aa, Artificial Sequence
  synthetic substrate for kinase
AAAGGGGGRPRAATFAE

SEQ ID NO: 2 - protein, 14 aa, Artificial Sequence
  synthetic substrate for kinase
AAARARTSSFAEPG

(19) United States
(12) Patent Application Publication
(10) Pub. No.: US 2010/0298377 A1
(43) Pub. Date: Nov. 25, 2010

(54) NOVEL SUBSTITUTED INDAZOLES, THE PREPARATION THEREOF AND USE OF SAME IN THERAPEUTICS

(75) Inventors: Michel ALETRU, Paris (FR); Dominique DAMOUR, Orly (FR); Patrick MOUGENOT, Paris (FR); Frederico NARDI, Paris (FR); Patrick NEMECEK, Orveau (FR); Christophe PHILIPPO, Gif sur Yvette (FR); Catherine MONSEAU, Le Vesinet (FR); Claudie NAMANE, Villiers-sur-Marne (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/636,357

(22) Filed: Dec. 11, 2009

Related U.S. Application Data
(63) Continuation of application No. PCT/FR2008/000843, filed on Jun. 18, 2008.

(30) Foreign Application Priority Data
Jun. 21, 2007 (FR) 0704422

(51) Int. Cl. A61K 31/4545; C07D 401/10; A61K 31/454; C07D 401/14; A61P 35/00

(52) U.S. Cl. 514/318; 546/199; 514/322; 546/194

(57) ABSTRACT

This disclosure relates to compounds of formula (I):

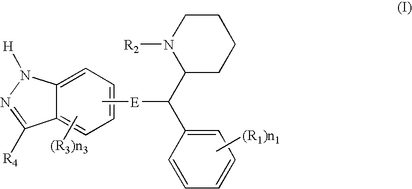

wherein R₁, R₂, R₃, R₄, E, and n₁ are as defined in the disclosure, to compositions containing them, to processes for preparing them, and the use thereof.

This application is a continuation of International Application No. PCT/FR2008/000843, filed Jun. 18, 2008, which is incorporated herein by reference in its entirety; which claims the benefit of priority of French Patent Application No. 0704422, filed Jun. 21, 2007.

The present invention relates to novel chemical compounds of the substituted indazole type, to compositions containing them and also to their use as medicaments, especially anti-cancer medicaments. The invention also relates to the process for preparing these compounds and to certain reaction intermediates.

### TECHNICAL FIELD

To date, most of the commercial compounds used in chemotherapy have major problems of side effects and of patient tolerance. The search for novel anti-cancer agents has been directed in recent years towards treatments targeting enzymes or other biomolecules predominantly expressed and/or activated in cancer cells. One major class of enzymes that has formed the subject of numerous studies is the family of protein kinases.

Protein kinases are a family of enzymes that catalyse the phosphorylation of hydroxyl groups of specific residues of proteins such as tyrosine, serine or threonine residues. Such phosphorylations can widely modulate the function of proteins; thus, protein kinases play a major role in regulating a wide variety of cell processes, especially including metabolism, cell proliferation, cell differentiation, cell migration or cell survival. Among the various cellular functions in which the activity of a protein kinase is involved, certain processes represent attractive targets for treating cancer diseases and also other diseases.

AGC denotes the group of cAMP-dependent protein kinases/G protein kinases/C protein kinases. The AGC subfamily of kinases phosphorylates its substrates on serine and threonine residues and participates in numerous well-known signalling pathways, such as the cyclic AMP (cAMP) signalling pathways, diacylglycerol signalling, the response to insulin and to other growth factors, apoptosis and the control of protein translation (Peterson et al., Curr. Biol. 1999, 9, R521). This AGC subfamily includes the proteins ROCK, PKA, PKB, PKC, PRK, P70S6K, SGK, RSK, GRK, MSK, PDK1 and PKG.

The ribosomal protein kinases p70S6K (1 and 2) belong to the AGC subfamily. The kinases p70S6K catalyse the phosphorylation of various substrates, and in particular the phosphorylation and activation of the ribosomal protein S6 that is involved in the positive regulation of the translation of the TOP mRNAs. These mRNAs contain an extended oligopyrimidine at the 5′ end, known as 5′TOP, and code for essential components of the protein translation machinery (Volarevic et al. Prog. Nucleic Acid Res. Mol. Biol. 2001, 65, 101-186). Phosphorylation of the ribosomal protein S6 is directly associated with regulation of the size of cells. p70S6K is activated in response to numerous extracellular signals including the nutrient pathway and the pathway of translation of the signal from the receptors of growth factors PI3K/mTOR (Hay and Sonenberg Genes Dev. 2004, 18, 1926-1945). The protein p70S6K is activated and/or amplified in various types of cancer, especially including breast cancer, thyroid cancers and cancers presenting mutations that inactivate the tumour suppressors TSC1 and/or TSC2 (Miyakawa et al. Endocrin. J. 2003, 50, 77-83; Van der Hage et al., Br J. Cancer 2004, 90, 1543-50; McManus E. J. & Alessi D. R. Nature Cell Biol. 2002, 4, E241-E216). The key role of p70S6K in human cancers is also demonstrated by the clinical inhibition of activation of p70S6K observed in kidney carcinoma in the case of patients treated with the inhibitor of mTOR CCI-779 (rapamycin ester): a significant linear association between the progress of the disease and inhibition of the activity of p70S6K has been reported (Peralba et al. Clinical Cancer Research 2003, 9, 2887-2892).

The protein kinase AKT (also known as PKB or Rac-PK β) also belongs to the AGC subfamily. It is a kinase of the subfamily of serine/threonine kinases (Hemmings, Science 1997, 275, 628). Three isoforms of human AKT, showing very strong homology between them, have been reported, AKT-1, -2 and -3, also known as PKBα, PKBβ and PKBγ (Cheng et al., Proc. Natl. Acad. Sci. USA 1992, 89, 9267-9271).

The PI3K/AKT pathway is activated via numerous factors, such as growth factors, for instance platelet-derived growth-factor and the growth factor IGF-1 (Insulin-like Growth Factor). The activation of PI3K increases the levels of phosphatidylinositol (3,4,5)-triphosphate (PIP3) at the plasma membrane, and thus promotes the recruitment of AKT at the membrane via its PH domain (pleckstrin homology). This enables its activation via phosphorylation with PDK1 on T308, T309 and T305 and via PDK2 on S473, S474 and S472 for the three isoforms AKT-1, -2 and -3, respectively (Hemmings, Science 1997, 275, 628; Bellacosa et al., Oncogene 1998, 17, 313-325). Several candidate kinases have been proposed for PDK2, including, recently, the mTOR-Rictor complex (Sarbassov dos D et al., Science 2005, 307, 1098-1101).

AKT plays a key role in transduction of the extracellular signals originating especially from receptors of growth factors with tyrosine kinase activity, via PI3K. By phosphorylating a wide variety of substrates, AKT is involved in numerous cell functions, including the survival and proliferation of cells, protein translation, angiogenesis, chemoresistance and radioresistance (Alessi et al., Curr. Opin. Genet. Dev. 1998, 8, 55-62).

Genetic anomalies in the PI3K/AKT pathway are common in human cancers and play an important role in cell transformation. In particular, the frequent deficiency of phosphatase PTEN, which is a negative regulator of the pathway, in a very large number of human cancers, induces the constitutive activation of AKT. Inactivating mutations or deletions of PTEN have been reported in a wide variety of tumour types, including glioblastomas, melanomas and breast, prostate, kidney and endometrial tumours. AKT-2 has been found genetically amplified in human ovarian, breast and pancreatic carcinomas (Testa J R. and Bellacosa A. Proc. Natl. Acad. Sci. USA 2001, 98, 10983-10985; Cheng et al., Proc. Natl. Acad. Sci. USA 1992, 89, 9267-9271; Bellacosa et al., Int. J. Cancer 1995, 64, 280-285; Cheng et al., Proc. Natl. Acad. Sci. USA 1996, 93, 3636-3641; Yuan et al. Oncogene 2000, 19, 2324-2330). Amplifications of AKT-1 have been found in human gastric cancers (Staal et al., Proc. Natl. Acad. Sci. USA 1987, 84, 5034-5037). The kinase activity of AKT-1 is found to be increased in prostate and breast cancers, and this is associated with a poor prognosis (Sun et al. Am. J. Pathol. 2001, 159, 431-437). The kinase activity of AKT-3 is found to be increased in various types of cancer, especially including breast cancers deficient in oestrogen receptors, and androgen-insensitive prostate cancers (Nakatani et al. J. Biol. Chem. 1999, 274, 21528-21532).

The experimental results indicate that the AKT protein kinases play a key role in the biology of a very large number of tumours and in particular in diseases presenting genetic anomalies of the PI3K/AKT signal transduction pathway. Consequently, the selective inhibition of one or more AKT isoenzyme(s) appears to be a promising approach for the treatment of cancer. Blocking the AKT kinase should inhibit the proliferation of tumour cells, make them sensitive to apoptosis and make them more sensitive to chemotherapy and radiotherapy.

The resistance of many types of cancer to conventional chemotherapy is a major factor limiting the success of cancer treatments; the PI3K/AKT pathway might be targeted to overcome chemotherapeutic resistance (McCormick Nature 2004, 428, 267-269; Bellacosa et al. Canc. Biol. Therap. 2004, 3, 268-275; West et al., Drug Resistance Update 2002, 5, 234-248; Bianco et al. Oncogene 2003, 22, 2812-2822). Thus, conventional targeted cytotoxic and anti-angiogenic antiproliferative therapies could complement the pro-apoptotic mechanism of an AKT inhibitor.

Thus, it is particularly desirable to have available novel protein kinase inhibitors, especially of the AGC kinases. In this respect, AKT (PKB) and/or S6K inhibitors would be most particularly advantageous. Such inhibitors might prove to be particularly useful in cancer treatment as antiproliferative, apoptosis-inducing, anti-metastasic, anti-invasive, anti-angiogenic, radio-sensitizing and chemo-sensitizing agents. Moreover, it is desirable to have available novel combinations including the compounds of the present invention and an inhibitor of other kinases of the PI3K/AKT pathway, such as IGF1R, PI3K, PDK1, mTOR and EIF4A.

International patent application WO 02/10137 describes compounds of formula (a):

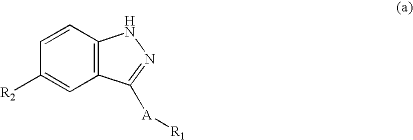

for which A may represent a single bond, (CH₂)ₐ, (CH₂)_{b}CH═CH(CH₂)_{c} or (CH₂)_{b}C≡C(CH₂)_{c} and R₁ an aryl or heteroaryl group or a heterocycle fused to a phenyl. R₂ may represent, among other possibilities, a group —(CH₂)_{b}NR₆C(═O)NR₆R₇, R₆ and R₇ possibly being an alkyl, aryl, arylalkyl, heterocycle or heterocycloalkyl optionally substituted with 1 to 4 groups R₃ which may be a halogen atom or an OH, carboxyl, alkyl, alkoxy, haloalkyl, acyloxy, . . . , aryl, substituted aryl, arylalkyl, heterocycle, substituted heterocycle, etc. group.

International patent application WO 03/078402 describes anti-cancer compounds of formula (b):

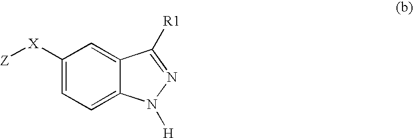

for which X represents SO₂NH, SO₂O, NHSO₂ or OSO₂ on the indazole ring and Z is an optionally substituted alkyl, aryl, heteroaryl, heterocycloalkyl or cycloalkyl group. The preceding unit is therefore not described either.

International patent application WO 2006/135383 describes antiviral compounds of formula (c):

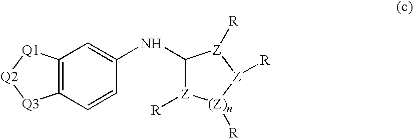

In the abovementioned patent applications, the following unit that characterizes the compounds of the invention:

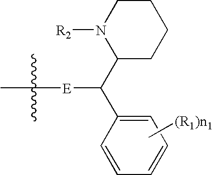

is not described. In particular, for WO 02/10137, the preceding unit results from a multiple selection for the group R₂.

International patent application WO 2005/037792 describes compounds of formula (d) pertaining to a different therapeutic field (treatment of behavioral disorders, psychoses, forms of anxiety, phobias, etc.):

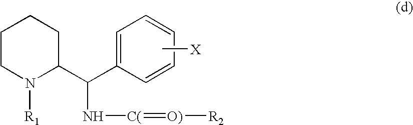

R₂ may be a naphthyl, pyridyl, pyrimidinyl, pyrazinyl, pyridazinyl, benzimidazolyl, indazolyl, triazolyl, etc. group. The group E and position 5 or 6 on the indazole nucleus are not described therein.

The article in Current Opinion in Drug Discovery & Development 2002, Vol. 5, No. 5, pp. 718-727 describes kinase-inhibiting compounds containing a urea bond between the rings.

None of these documents describes the compounds according to the invention.

### DESCRIPTION OF THE INVENTION

### General Definitions

In the context of the present invention, the following definitions are used in the text, unless otherwise mentioned:
halogen atom: a fluorine, chlorine, bromine or iodine atom;
alkyl group: a linear or branched, saturated aliphatic hydrocarbon-based group, preferably containing from 1 to 20 carbon atoms and preferably from 1 to 5 carbon atoms. Mention may be made especially of the following groups: methyl, ethyl, propyl, butyl, pentyl, hexyl, octyl, nonyl, decyl, dodecyl, hexadecyl, octadecyl, isopropyl, isobutyl, tert-butyl, 2-ethylhexyl,

2-methylbutyl, 2-methylpentyl, 1-methyl pentyl and 3-methylheptyl;
alkenyl group: an alkyl group comprising one or more C═C double bonds. Mention may be made of the following groups: allyl, pentenyl, hexenyl, octenyl;
alkynyl group: an alkyl group comprising one or more C═C triple bonds. Mention may especially be made of the following groups: hexynyl, heptynyl, octynyl;
cycloalkyl group: a cyclic alkyl group containing from 3 to 10 carbon atoms engaged in the ring structure. Mention may especially be made of the following groups: cyclopropyl, cyclopentyl, cyclohexyl;
aryl group: mono- or bicyclic aromatic group of 6 to 10 carbon atoms. Mention may especially be made of the following groups: phenyl, naphthyl, indenyl, fluorenyl;
heteroaryl group: a 5- to 10-membered mono- or bicyclic aromatic group comprising, as atoms forming the ring, one or more heteroatoms chosen from O, S and N. Mention may especially be made of the following groups: pyrazinyl, thienyl, oxazolyl, furazanyl, pyrrolyl, 1,2,4-thiadiazolyl, naphthyridinyl, pyridazinyl, quinoxalinyl, phthalazinyl, imidazo[1,2-a]pyridyl, imidazo[2,1-b]thiazolyl, cinnolinyl, triazinyl, benzofurazanyl, azaindolyl, benzimidazolyl, benzothienyl, thienopyridyl, thienopyrimidinyl, pyrrolopyridyl, imidazopyridyl, benzazaindolyl, 1,2,4-triazinyl, benzothiazolyl, furyl, imidazolyl, indolyl, triazolyl, tetrazolyl, indolizinyl, isoxazolyl, isoquinolyl, isothiazolyl, oxadiazolyl, pyrazinyl, pyridazinyl, pyrazolyl, pyridyl (Pyr), pyrimidinyl, purinyl, quinazolinyl, quinolyl, isoquinolyl, 1,3,4-thiadiazolyl, thiazolyl, triazinyl, isothiazolyl, carbazolyl;
heterocycloalkyl group: a cycloalkyl group as defined above also comprising, as atoms forming the ring, one or more heteroatoms chosen from N, O and S. Among these, mention may be made especially of epoxyethyl, oxiranyl, aziridinyl, tetrahydrofuryl, dioxolanyl, pyrrolidinyl, pyrazolidinyl, imidazolidinyl, tetrahydrothiophenyl, dithiolanyl, thiazolidinyl, tetrahydropyranyl, dioxanyl, morpholinyl, piperidyl, piperazinyl, tetrahydrothiopyranyl, dithianyl, thiomorpholinyl, dihydrofuryl, 2-imidazolinyl, 2,-3-pyrrolinyl, pyrazolinyl, dihydrothiophenyl, dihydropyranyl, pyranyl, tetrahydropyridyl, dihydropyridyl, tetrahydropyrimidinyl, dihydrothiopyranyl, and the corresponding groups derived from fusion with a phenyl nucleus, and more particularly epoxyethyl, oxiranyl, tetrahydrofuryl, dioxolanyl, pyrrolidinyl, tetrahydropyranyl, dioxanyl, morpholinyl, piperidyl, piperazinyl, tetrahydrothiopyranyl, and more particularly tetrahydropyranyl rings.

For all the compounds described in the present invention, the alkyl, alkenyl, alkynyl, aryl, heteroaryl, heterocycloalkyl and cycloalkyl groups mentioned above may be optionally substituted with one or more substituents. These substituents may be chosen from halogen atoms and alkyl, alkenyl, alkynyl, aryl, CN, NRR′, CF₃, OR′, COOR, CONRR′, COR, heteroaryl, heterocycle, cycloalkyl or —SO₂NRR′ groups, these substituents themselves possibly being substituted with one or more substituents chosen from halogen atoms and alkyl, alkenyl, alkynyl, aryl, CN, NRR′, CF₃, OR, COOR, CONRR′, COR, heteroaryl, heterocycle, cycloalkyl or —SO₂NRR′ groups.

According to a first aspect, the invention relates to compounds of formula (I):

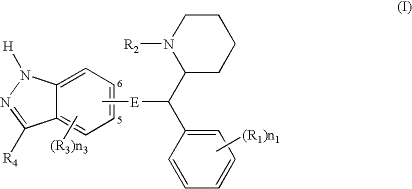

E denotes a group of formula —NT-CO—O— or —NT-CX-NT′- attached in position 5 or 6 via —NT- to the indazole nucleus, in which X denotes ═O or ═S and T and T′, which may be identical or different, are chosen independently from H and an alkyl group. E may more particularly be one of the following groups: —NH—CO—O—, —NH—CO—NH—, —NH—CS—NH—, —NH—CO—N-alkyl-, preferably —NH—CO-NMe-, or —N-alkyl-CO—NH—, preferably —NMe-CO—NH—. Preferentially, E denotes —NH—CO—O— (—NH— being attached to the indazole nucleus).

E may also be in the form of a 5- or 6-membered ring of formula

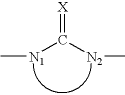

(attached to the indazole nucleus via the nitrogen atom N₁). For example, E may be one of the following groups:

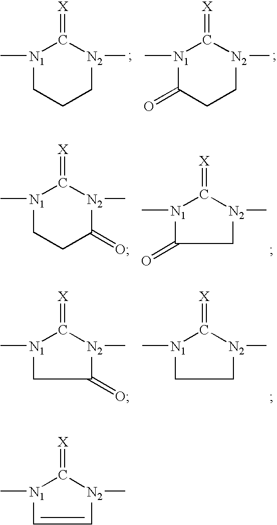

R₁ represents one or more substituents, chosen independently from each other when there are several, from: a halogen atom, an alkyl, alkenyl, alkynyl, haloalkyl, haloalkoxy, aryl, heteroaryl, heterocycloalkyl, cycloalkyl, CN, NRR′, OR, NO₂, COOR, CONRR′, NRCOR′ group. R and R′, which may be identical or different, denote, independently of each other, a hydrogen atom or an alkyl, aryl, heterocycloalkyl, cycloalkyl or heteroaryl group.

As alkyl group, R₁ may especially be a group CH₂NHR. As alkynyl group, R₁ may be a group —C≡C—R. As aryl group, R₁ may be a phenyl group (Ph), optionally substituted with at least one substituent, for example chosen from —CH₂OR, —NHCOR, —NHCH₂R. As group —COOR, R₁ may especially be —COOH or COOalkyl (for example COOEt). As group —CONHR, R₁ may especially be —CONHPh or —CONH—C—C₆H₁₁, a phenyl group possibly being optionally substituted, for example R₁ may be —CONH(4-^{t}Bu)Ph. As group haloalkyl, R₁ may especially be —CF₃. As haloalkoxy group, R₁ may especially be —OCF₃. R₁ may be one of those described in Table I.

R₂ represents a hydrogen atom or an alkyl, alkenyl or alkynyl group. R₂ may be for example a methyl or allyl group —CH₂—CH═CH₂. R₂ may be one of those described in Table I.

R₃ represents one or more substituents, chosen independently from each other when there are several, from: a halogen atom, an alkyl, alkenyl, alkynyl, haloalkoxy (for example —OCF₃), aryl, heteroaryl, cycloalkyl, heterocycloalkyl, —CN, —NRR′, —CF₃, —OR, —NO₂, —COOR, —CONRR′, —NRCOR′ group. R₃ more particularly denotes a halogen atom, especially fluorine, or an alkyl group, especially a methyl group. R₃ may be one of those described in Table I.

R₄ denotes a hydrogen or halogen atom or an alkyl, alkenyl, alkynyl, aryl, heteroaryl, heterocycloalkyl, —NR—CO—R′, —COOR, —NRR′, —CHO or —CONR(OR′) group. R₄ may be one of those described in Table I.

As alkyl group, R₄ may be a methyl or —CH₂CH₂Ph group.

As alkynyl group, R₄ may be —C≡C—R, R denoting an aryl or heteroaryl group. The aryl group is more particularly a phenyl group, optionally substituted with a fluorine atom in position 3 or 4. The heteroaryl group may be a 3-pyridyl group.

As aryl group, R₄ may be a phenyl group, optionally substituted with a group —SO₂NH₂ in position 4.

As heteroaryl group, R₄ may especially be a 2-, 3- or 4-pyridyl group or a benzimidazolyl group.

As group —NR—CO—R′, R₄ may be the group —NH—CO-Ph.

As group —NRR′, R₄ may be —NH₂.

n₁ represents an integer ranging from 0 to 5 and n₃ an integer ranging from 0 to 3 (when n₁ and/or n₃=0, there are no other substituents). Preferably, n₁=0, 1 or 2 and/or n₃=0 or 1.

Mention is made of the compounds of formula (I) for which:
R₁ represents one or more substituents, chosen independently from each other when there are several, from: a halogen atom and a group —COOR or —CONRR′;
R₂ represents a hydrogen atom or an alkyl or alkenyl group;
R₃ represents a halogen atom;
R₄ denotes a hydrogen or halogen atom or an alkyl, aryl, heteroaryl, —NR—CO—R′ or —NRR′ group;
R and R′, which may be identical or different, denote, independently of each other: a hydrogen atom or an alkyl or aryl group;
n₁ represents an integer ranging from 0 to 5 and n₃ an integer ranging from 0 to 3.

Among the compounds of formula (I), a first subgroup is distinguished for which n₁=0, R₂ represents a hydrogen atom or an alkyl or alkenyl group, n₃=0 and R₄ represents a hydrogen atom or an alkyl group. R₂ may be an alkyl or alkenyl group, for example methyl or allyl, and R₄ a hydrogen atom. R₂ and R₄ may both be an alkyl group, for example, respectively, methyl and methyl, or alternatively methyl and —CH₂CH₂Ph.

Among the compounds of formula (I), a second subgroup is distinguished for which R₁ represents a halogen atom, R₂ represents a hydrogen atom or an alkyl group, n₃=0 and R₄ represents a hydrogen atom. Preferably, n₁=2 and R₁ represents a fluorine and/or chlorine atom. R₂ may be a methyl group.

Among the compounds of formula (I), a third subgroup is distinguished for which n₁=0, R₂ represents a hydrogen atom or an alkyl group, n₃=0 and R₄ represents an aryl or heteroaryl group. As aryl group, R₄ may be a phenyl group, optionally substituted with a group —SO₂NH₂ in position 4. As heteroaryl group, R₄ may be may be a 2-, 3- or 4-pyridyl group or a benzimidazolyl group. R₂ may be a methyl group.

Among the compounds of formula (I), a fourth subgroup is distinguished for which n₁=0, R₂ represents a hydrogen atom or an alkyl group, n₃=0 and R₄ represents a group —C≡C—R, R denoting an aryl or heteroaryl group. As aryl group, R may be a phenyl group, optionally substituted with a fluorine atom in position 3 or 4. As heteroaryl group, R may be a 3-pyridyl group. As group alkyl, R₂ may be a methyl group.

Among the compounds of formula (I), a fifth subgroup is distinguished for which n₁=0, R₂ represents a hydrogen atom or an alkyl group, R₃ represents a halogen atom or an alkyl group and R₄ represents a hydrogen atom or a group —NRR′ or —NR—CO—R. As group —NRR′, R₄ may be —NH₂. As group —NR—CO—R′, R₄ may be a group —NH—CO-Ph. Preferably, n₃=1. R₂ may be a methyl group. R₃ may be a fluorine atom.

Among the compounds of formula (I), a sixth subgroup is distinguished for which R₁ is chosen from: an alkyl, alkenyl, alkynyl, haloalkyl, haloalkoxy, heteroaryl, heterocycloalkyl, cycloalkyl, —CN, —NRR′, —OR, —NO₂, —COOR, —CONRR′ or —NRCOR′ group, R₂ represents a hydrogen atom or an alkyl group, n₃=0 and R₄ represents a hydrogen atom. Preferably, n₁=1. R₂ may be a methyl group. R₁ may be one of the following groups: 3-COOEt, 3-CONH-(4-^{t}Bu)Ph or 3-CONHPh.

Among the compounds of formula (I), a seventh subgroup is distinguished for which n₁=0, 1 or 2 and R₁ represents a halogen atom, R₂ represents a hydrogen atom or an alkyl group, n₃=0 or 1 and R₃ represents a halogen atom, R₄ represents —NH₂, —CH₂CH₂Ph, a hydrogen atom or an alkyl, aryl (preferably a phenyl substituted with —SO₂NH₂ in position 4), heteroaryl (preferably a 2, 3 or 4-pyridyl or benzimidazolyl group) or —C≡C—R group, R denoting an aryl (preferably a phenyl group, optionally substituted with a fluorine atom in position 3 or 4) or heteroaryl (preferably a 3-pyridyl group) group.

For all the compounds described, E more particularly denotes —NH—CO—O— or —NH—CO—NH— and is attached via the —NH— to the indazole. Preferably also, it is attached in position 5.

More preferentially, the compounds may be chosen from the following list:
1-(1H-indazol-5-yl)-3-[(1-methylpiperid-2-yl)(phenyl)methyl]urea((S,2S),(R,2R));
1-(1H-indazol-6-yl)-3-[(1-methylpiperid-2-yl)(phenyl)methyl]urea((S,2S),(R,2R));
1-{(S)-(3-chloro-5-fluorophenyl)[(2S)-piperid-2-yl]methyl}-3-(1H-indazol-5-yl)urea;
1-{(S)-(3,4-dichlorophenyl)[(2S)-piperid-2-yl]methyl}-3-(1H-indazol-5-yl)urea;
1-[(3-chloro-5-fluorophenyl)(piperid-2-yl)methyl]-3-(1H-indazol-5-yl)urea;
1-{(R)-(3,4-dichlorophenyl)[(2R)-piperid-2-yl]methyl}-3-(1H-indazol-5-yl)urea;
1-{(S)-(3,5-dichlorophenyl)[(2S)-piperid-2-yl]methyl}-3-(1H-indazol-5-yl)urea;
1-{(S)-(phenyl)[(2S)-piperid-2-yl]methyl}-3-(1H-indazol-5-yl)urea;
1-{(R)-(phenyl)[(2R)-piperid-2-yl]methyl}-3-(1H-indazol-5-yl)urea;
1-{(S)-(phenyl)[(2S)-1-allylpiperid-2-yl]methyl}-3-(1H-indazol-5-yl)urea;
1-[(3-chloro-5-fluorophenyl)(1-methylpiperid-2-yl)methyl]-3-(1H-indazol-5-yl)urea;
4-[5-({[(1-methylpiperid-2-yl)(phenyl)methyl]carbamoyl}amino)-1H-indazol-3-yl]benzenesulfonamide((S,2S),(R,2R));
1-[(1-methylpiperid-2-yl)(phenyl)methyl]-3-(3-pyrid-4-yl-1H-indazol-5-yl)urea((S,2S),(R,2R));
1-[(1-methylpiperid-2-yl)(phenyl)methyl]-3-(3-pyrid-3-yl-1H-indazol-5-yl)urea((S,2S),(R,2R));
1-[(1-methylpiperid-2-yl)(phenyl)methyl]-3-(3-pyrid-2-yl-1H-indazol-5-yl)urea((S,2S),(R,2R));
1-{(S)-(3,4-dichlorophenyl)[(2S)-piperid-2-yl]methyl}-3-(3-pyrid-4-yl-1H-indazol-5-yl)urea;
1-[3-(1H-benzimidazol-2-yl)-1H-indazol-5-yl]-3-[(1-methylpiperid-2-yl)(phenyl)methyl]urea((S,2S),(R,2R));
3-methyl-5-({[(1-methylpiperid-2-yl)(phenyl)methyl]carbamoyl}amino)-1H-indazole((S,2S),(R,2R));
1-(3-amino-7-fluoro-1H-indazol-5-yl)-3-[(1-methylpiperid-2-yl)(phenyl)methyl]urea((S,2S),(R,2R));
1-(7-fluoro-1H-indazol-5-yl)-3-[(1-methylpiperid-2-yl)(phenyl)methyl]urea((S,2S),(R,2R));
1-{3-[(3-fluorophenyl)ethynyl]-1H-indazol-5-yl}-3-[(1-methylpiperid-2-yl)(phenyl)methyl]urea((S,2S),(R,2R));
1-[(1-methylpiperid-2-yl)(phenyl)methyl]-3-[3-(phenylethynyl)-1H-indazol-5-yl]urea((S,2S),(R,2R));
1-{3-[(4-fluorophenyl)ethynyl]-1H-indazol-5-yl}-3-[(1-methylpiperid-2-yl)(phenyl)methyl]urea((S,2S),(R,2R));
1-[(1-methylpiperid-2-yl)(phenyl)methyl]-3-[3-(pyrid-3-ylethynyl)-1H-indazol-5-yl]urea((S,2S),(R,2R));
1-{(S)-(3,4-dichlorophenyl)[(2S)-piperid-2-yl]methyl}-3-[3-(phenylethynyl)-1H-indazol-5-yl]urea;
1-[(1-methylpiperid-2-yl)(phenyl)methyl]-3-[3-(2-phenylethyl)-1H-indazol-5-yl]urea((S,2S),(R,2R));
N-[5-({[(1-methylpiperid-2-yl)(phenyl)methyl]carbamoyl}amino)-1H-indazol-3-yl]benzamide((S,2S),(R,2R));
1-(3-amino-1H-indazol-5-yl)-3-{(S)-(3,4-dichlorophenyl)[(2S)-piperid-2-yl]methyl}urea;
1-(1H-indazol-5-yl)-3-[(1-methylpiperid-2-yl)phenylmethyl]-1,3-dihydroimidazol-2-one((S,2S),(R,2R));
1-{(S)-(3,4-dichlorophenyl)[(2S)-piperid-2-yl]methyl}-3-(1H-indazol-5-yl)thiourea;
(3,5-dichlorophenyl)[piperid-2-yl]methyl 1H-indazol-5-ylcarbamate((S,2S),(R,2R));
(S)-(3-chloro-5-fluorophenyl)[(2S)-piperid-2-yl]methyl 1H-indazol-5-ylcarbamate;
(3-chloro-5-fluorophenyl)(1-methylpiperid-2-yl)methyl 1H-indazol-5-ylcarbamate;
(R)-(3,4-dichlorophenyl)[(2R)-piperid-2-yl]methyl 1H-indazol-5-ylcarbamate;
(S)-(3,4-dichlorophenyl)[(2S)-piperid-2-yl]methyl 1H-indazol-5-ylcarbamate;
(R)-(3,4-dichlorophenyl)[(2S)-piperid-2-yl]methyl 1H-indazol-5-ylcarbamate;
(S)-(3-chloro-5-fluorophenyl)[(2R)-piperid-2-yl]methyl 1H-indazol-5-ylcarbamate;
(R)-(3-chloro-5-fluorophenyl)[(2R)-piperid-2-yl]methyl 1H-indazol-5-ylcarbamate;
[3-(anilinocarbonyl)phenyl][(2S)-piperid-2-yl]methyl 1H-indazol-5-ylcarbamate;
(S)-(3-trifluoromethylphenyl)[(2S)-piperid-2-yl]methyl 1H-indazol-5-ylcarbamate;
(S)-(3-iodophenyl)[(2S)-piperid-2-yl]methyl 1H-indazol-5-ylcarbamate;
(S)-(3-bromophenyl[(2S)-piperidyl]methyl 1H-indazolylcarbamate;
(S)-(3-cyclohexylcarbamoyl-phenyl)-(S)-piperid-2-ylmethyl(1H-indazol-5-yl)carbamate;
(7-fluoro-1H-indazol-5-yl)-(S)-(3,4-dichlorophenyl)-(S)-piperid-2-ylmethyl carbamate;
(7-fluoro-1H-indazol-5-yl)-(S)-(3-chloro-5-fluorophenyl)-(S)-piperid-2-ylmethyl carbamate; and
(6-fluoro-1H-indazol-5-yl)-(S)-(3-chloro-5-fluorophenyl)-(S)-piperid-2-ylmethyl carbamate.

The compounds according to the invention may comprise at least two asymmetric carbons, and may thus exist in the form of enantiomers or diastereoisomers. These enantiomers and diastereoisomers, and also mixtures thereof, also form part of the invention. The compounds according to the invention may also exist in the form of hydrates or solvates, i.e. in the form of associations or combinations with one or more molecules of water or of a solvent. These hydrates and solvates also form part of the invention. The compounds according to the invention may also exist in the form of salts, i.e. of addition compounds of the compounds according to the invention with organic or mineral acids or bases. These are more specifically referred to as “pharmaceutically acceptable salts” when the acids or bases are non-toxic and allow the pharmacological properties of the compounds according to the invention to be preserved. The salts are prepared according to the techniques known to those skilled in the art (see, for example, H. Ansel et al., Pharmaceutical Dosage Forms and Drug Delivery Systems, 6th edition, 1995, pp. 196 and 1456-1457). The compounds according to the invention may also, where appropriate, be in various tautomeric forms, which are included in the invention. Thus, the compounds according to the invention may be (i) in racemic form, or enriched in an enantiomer, and/or (ii) in salified form and/or (iii) in hydrated or solvated form.

According to a second aspect, the compounds according to the invention may be used for the preparation of a medicament, especially a medicament for preventing and/or treating a cancer (anti-cancer agent). The invention also relates to a medicament comprising a compound according to the invention.

According to a third aspect, the invention relates to a pharmaceutical composition comprising, as active principle, a compound according to the invention in combination with a pharmaceutically acceptable excipient (according to the chosen mode of administration). The dose of active principle administered will be adapted by the practitioner as a function of the route of administration to the patient and of the patient's condition.

The pharmaceutical composition may be in solid or liquid form or in the form of liposomes according to the chosen mode of administration. The solid forms are constituted of powders, gel capsules or tablets. The supports used for the solid forms are constituted especially of mineral or organic supports. The liquid forms are constituted of solutions, suspensions or dispersions.

The compounds of the present invention may be administered alone or as a mixture with at least one other anti-cancer agent. The latter agent may be chosen from:
chemotherapy agents such as alkylating agents, Pt derivatives, antibiotic agents, antimicrotubule agents, taxoids, anthracyclines, group I and II topoisomerase inhibitors, fluoropyrimidines, cytidine analogues, adenosine analogues, enzymes, and also oestrogen-based and androgenic hormones;
antivascular or anti-angiogenic agents;
other kinase inhibitors and in particular of kinases of the PI3K/AKT transduction pathway (cf. Rapamycin and analogue Temsirolimus), and also inhibitors of receptors with tyrosine kinase activity in particular with EGFR (cf. Tarceva), HER2 and IGFR, inhibitors of various signal transduction pathways, in particular with MAPK pathway inhibitors, for instance MEK1/2 inhibitors, and inhibitors of other pathways that play a key role in the cancerization process, for instance the Notch pathway;
inhibitors of other biomolecules such as histone deacetylase inhibitors, COX-2 inhibitors, MMP inhibitors and proteasome inhibitors.

It is also possible to combine the compounds according to the invention with a radiotherapy. This treatment may be administered simultaneously, separately or sequentially. The treatment will be adapted by the practitioner as a function of the disease to be treated. Combinations of the compounds of the invention with the agents mentioned above or with radiation form another subject of the present invention.

According to a fourth aspect, the invention relates to a process for preparing the compounds according to the invention. These compounds are obtained from the precursor compounds of formula (IA), (IB) or (IC), which are prepared according to one of the Schemes described below. In the Schemes that follow, A represents R₂ (except for H) or a protecting group PG₁. B represents H or a protecting group PG₂.

Depending on the nature of the compounds (IA), (IB) or (IC), one or more deprotection steps, optionally followed or preceded by an alkylation of NH to NR₂, are necessary to obtain the compounds of formula (I):
(i) if A=R₂ and B=H: no additional step is necessary;
(ii) if A=R₂ and B=PG₂: step of deprotection of PG₂ to H;
(iii) if A=PG₁ and B=H: step of deprotection of PG₁ to H, optionally followed or preceded by an alkylation reaction of NH to NR₂;
(iv) if A=PG₁ and B=PG₂: step of deprotection of PG₁ to H, step of deprotection of PG₂ to H, optionally followed or preceded by an alkylation reaction of NH to NR₂.

The protecting groups PG₁ and PG₂ are introduced in the protection step in order to avoid unwanted side reactions during one or more reaction steps, and they are removed during the deprotection step. Examples of protecting groups will be found in T. W. Greene et al. “Protective Groups in Organic Synthesis”, 3^{rd} edition, 1999, Wiley-Interscience or alternatively in J. F. W. McOmie “Protective Groups in Organic Chemistry”, Plenum Press, 1973. Examples of protecting groups that may be mentioned include tert-butyl carbamate (BOC), the allyl group —CH₂—CH═CH₂ or the [2-(trimethylsilyl)ethoxy]methyl (SEM) group. It is understood that, for some of the compounds of the invention, it may be necessary to introduce, into one or more steps of the synthesis, other groups for protecting other chemical functions. The two protecting groups PG₁ and PG₂ may be identical or different. The deprotection step consists in removing the protecting group(s) via application of suitable experimental conditions known to those skilled in the art.

Preparation of Compounds (IA) with E=-NT-CO—O— (Carbamate Function)

Compounds (IA) are prepared according to Scheme 1 via coupling of compounds P₁ and P₂ using an agent for introducing the unit C═O:

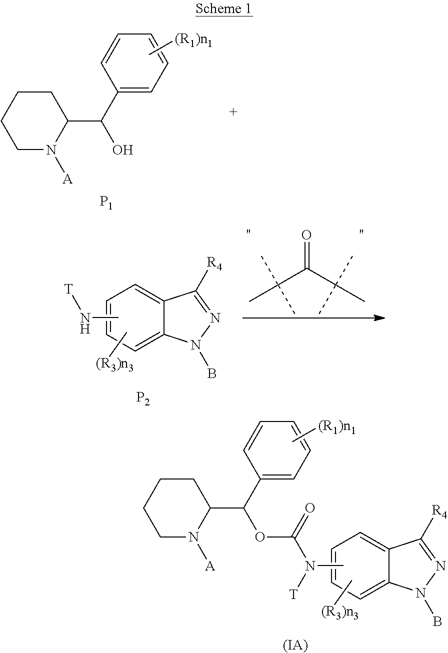

The agent may be, for example, phosgene, triphosgene or N,N′-disuccinimidyl carbonate. The reaction is performed in a solvent such as dichloromethane (DCM) or acetonitrile, preferably in the presence of a base (for example triethylamine). The temperature is preferably between 0° C. and the boiling point of the reaction medium. Preferably, P₁ is placed in contact first with the agent, and P₂ is then added.

Compounds P₁ may be prepared according to the teaching of WO 2003/089411 or according to the process of Scheme 2, starting with an aldehyde (1) and an organomagnesium reagent (2). The reaction is performed in an inert solvent (for example diethyl ether or tetrahydrofuran) at a temperature of between −70° C. and 20° C.:

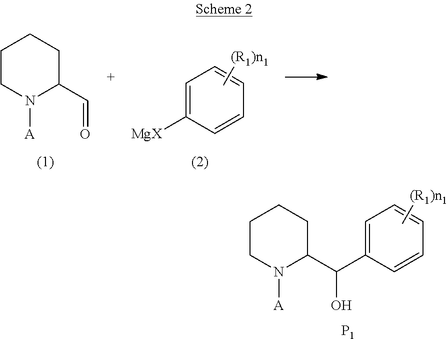

The aldehydes (1) may be commercially available or prepared by application for adaptation of the methods described by Molander, Gary A. et al. Tetrahedron 2005, 61(10), 2631-2643 or Yan, Lin et al., Bioorganic & Medicinal Chemistry Letters 2004, 14(19), 4861-4866 or Balboni, Gianfranco et al., European Journal of Medicinal Chemistry 2000, 35(11), 979-988 or Alibes, Ramon et al., Organic Letters 2004, 6(11), 1813-1816. The organomagnesium reagents (2) may be commercially available or prepared by application or adaptation of the usual methods known to those skilled in the art.

Preparation of Compounds (IB) with E=-NT-CX-NT′-

1st route: compounds (IB) are prepared according to Scheme 3 via coupling of compounds P₃ and P₂ using an agent for introducing the unit C═O or C═S:

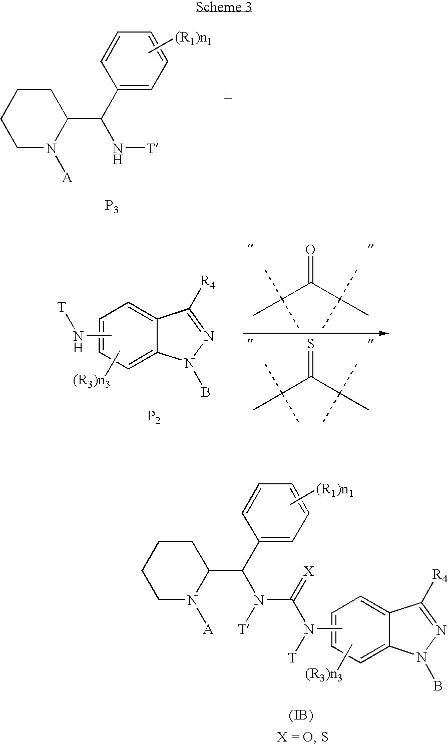

When X═O, the agent may be, for example, phosgene, triphosgene or N,N′-disuccinimidyl carbonate. The reaction is performed in a solvent such as DCM or acetonitrile, preferably in the presence of a base (for example triethylamine). The temperature is preferably between 0° C. and the boiling point of the reaction medium. When X═S, the agent may be carbon disulfide (CS₂). The reaction is performed in a solvent such as ethanol, preferably in the presence of a base such as potassium hydroxide. The temperature is between 0° C. and the boiling point of the reaction medium. Inspiration may also be taken from the methods described by Patel, H. et al., Indian Journal of Heterocyclic Chemistry 2006, 15(3), 217-220. Preferably, P₂ is first placed in contact with the agent, followed by addition of P₃.

2nd route: according to one variant, compounds (IB) for which X═O may also be obtained according to Scheme 4 via coupling of compounds P₃ and P′₂:

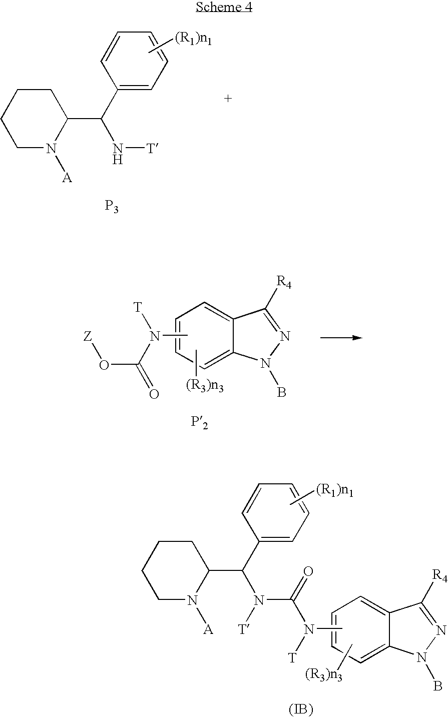

Z represents a phenyl group, optionally substituted with NO₂. The compounds P′₂ may be prepared from P₂ via conversion of the amine function —NHT of the compounds P₂ into a carbamate function —NT-CO—O—Z using chloroformate Z—O—COCl, preferably in the presence of a base (for example triethylamine). The conversion reaction is performed in a solvent such as THF at a temperature of between 0° C. and the boiling point of the reaction medium. It is not necessary to isolate the compounds P′₂ for the subsequent step. The coupling between compounds P′₂ and P₃ may be performed in a solvent such as acetonitrile or THF at a temperature of between 20° C. and the boiling point of the reaction medium. It is also possible to use microwaves for this final step, as described by Castelhano, Arlindo L. et al. Bioorganic & Medicinal Chemistry Letters 2005, 15(5), 1501-1504.

3rd route: compounds (IB) for which T=H may also be obtained via coupling of compounds P₃ and P₄ (Scheme 5). This reaction is performed in a solvent such as DCM at a temperature of between 0° C. and the boiling point of the reaction medium.

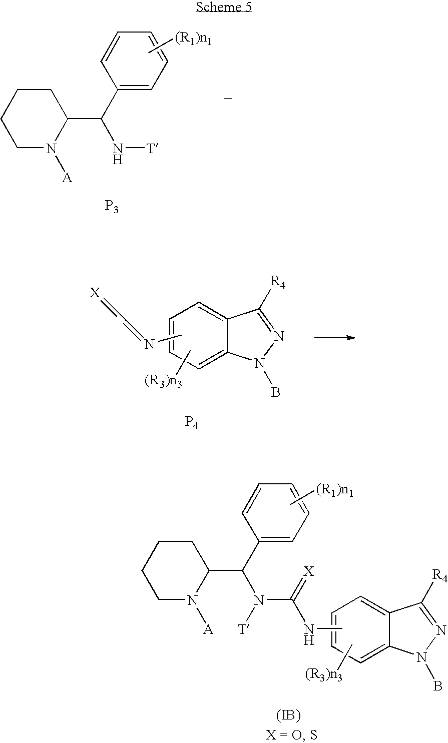

The compounds P₄ with X═O may be prepared by application of the methods described by I. Drizin et al., Bioorganic & Medicinal Chemistry 2006, 14(14), 4740-4749 and those with X═S by application of the methods described in WO 2002081453 or CH 605858.

The compounds P₃ with T′=H may be prepared according to the teaching of WO 2003/089411. They may also be prepared according to the process of Scheme 6 starting with the compounds P₁ for which A denotes an alkyl or allyl group, introduced via N-alkylation:

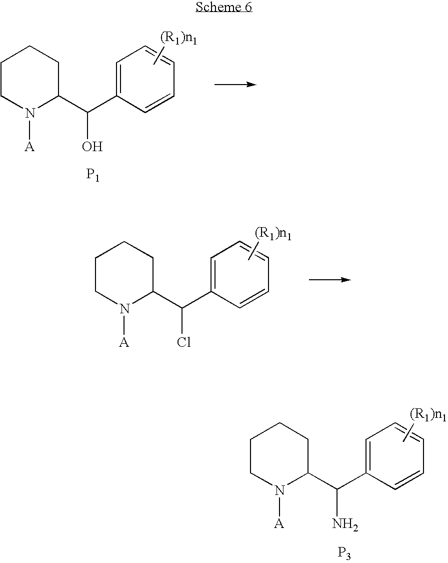

The N-alkylation is performed using a halide of A, in the presence of a base such as potassium carbonate, in a polar solvent such as acetonitrile at a temperature of between 0° C. and the boiling point of the reaction medium. The alcohol function of P₁ may then be converted into a nucleofugal group, for example a chlorine, via the action of mesyl chloride in the presence of potassium carbonate in a chlorinated solvent such as DCM at a temperature of between 0° C. and the boiling point of the reaction medium. The nucleofugal group is then reacted with aqueous ammonia dissolved in methanol, at a temperature of between 0° C. and the boiling point of the reaction medium.

The compounds P₃ with T′ other than H may be prepared via N-monoalkylation by application of the usual methods known to those skilled in the art. For example, the primary amine function NH₂ may be converted into a secondary amine NH-T′ (Scheme 7) via the action of a carbonyl derivative T″-CHO in the presence of a reducing agent such as lithium aluminium hydride (LiAlH₄) or sodium triacetoxyborohydride (NaBH(OAc)₃) in a solvent such as DMF or THF at a temperature of between 0° C. and the boiling point of the reaction medium.

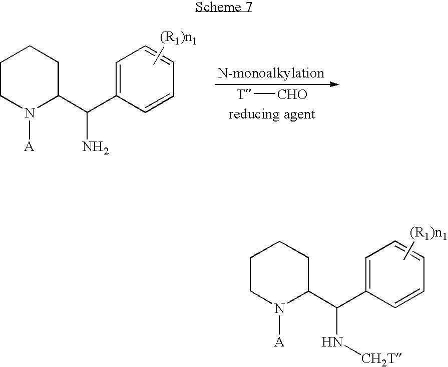

Preparation of Compounds (IC) with E in the Form of a 5- or 6-Membered Ring

Compounds (IC) may be prepared according to Scheme 8 via one of the three routes indicated. F and F′ represent two identical or different functions capable of reacting with an amine function to form the ring.

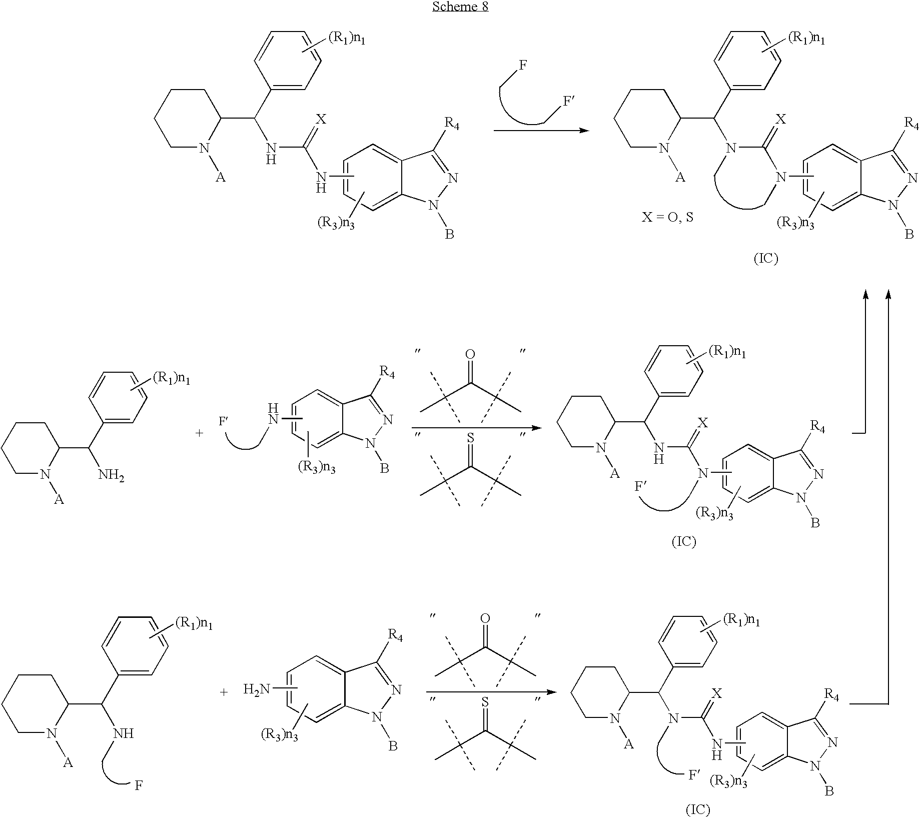

E:

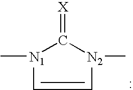

reference may be made to: WO 2006/136553 (cf. pages 17-18, reaction (XIII) [symbol] (V-B)) or WO 2005/121122 (cf. intermediates (X) and (XII)). The following intermediate compounds may be used:

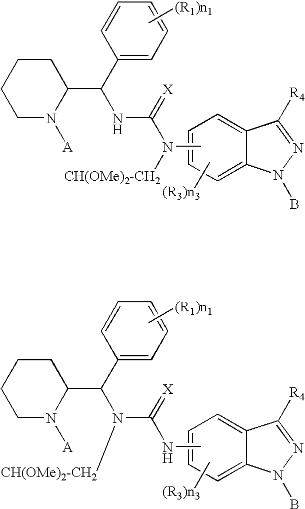

E:

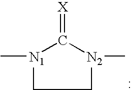

reference may be made to: Yasuda, Nobuyoshi et al., J. Org. Chem. 2004, 69(6), 1959-1966; Mayer, Patrice et al., J. Med. Chem. 2000, 43(20), 3653-3664; Yang, Dan et al. Organic Letters 2004, 6(10), 1577-1580. The preceding group E may thus be hydrogenated.

E:

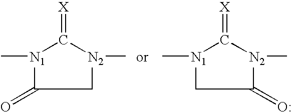

reference may be made to: Sigachev, Andrey S. et al., Journal of Heterocyclic Chemistry 2006, 43(5), 1295-1302; Randolph, John T. et al., Bioorganic & Medicinal Chemistry 2006, 14(12), 4035-4046; Kawato, Haruko C. et al. Organic Letters 2001, 3(22), 3451-3454.

E:

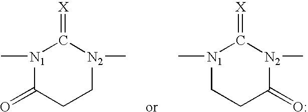

reference may be made to: Deck, L. M. et al., Journal of Heterocyclic Chemistry 2000, 37(4), 675-680; Lee, Chang Kiu et al., Bulletin of the Korean Chemical Society 1991, 12(3), 343-7; Soliman, Raafat et al., Journal of Pharmaceutical Sciences 1981, 70(8), 952-6,

E:

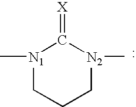

reference may be made to: Moustafa, Ahmed H. et al., Journal of Chemical Research 2005, 5, 328-331; Juaristi, Eusebio et al., Helvetica Chimica Acta 2002, 85(7), 1999-2008; Chapoteau, Eddy et al., J. Org. Chem. 1992, 57(10), 2804-8.

Preparation of the Indazoles

Compound P₂ with T=H may be obtained according to Scheme 9 via reduction of the corresponding nitro compound P₅:

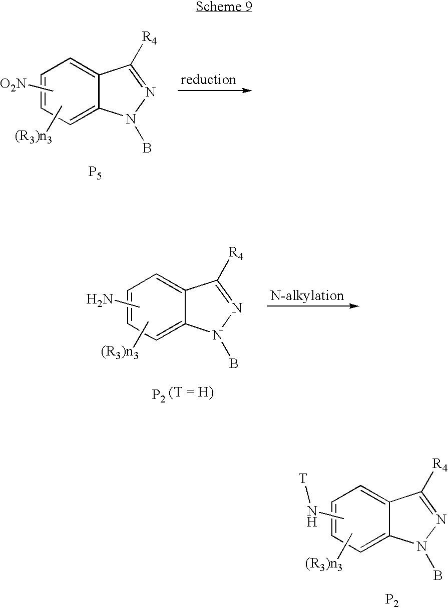

The reduction may be performed according to a method common to those skilled in the art, for example using ammonium formate in the presence of a palladium-on-charcoal catalyst (Ram, S. Tetrahedron Lett. 1984, 25, 3415), using ferrous sulfate (Castellano, S. J. Het. Chem. 2000, 37(6), 949) or using tin chloride, or using hydrogen in the presence of a catalyst such as palladium-on-charcoal or Raney nickel. Inspiration may also be taken from the reduction conditions given in the examples of patent application FR 2 836 914. Compound P₂ with T other than H is prepared via N-monoalkylation (cf. Scheme 7) using compound P₂ with T=H.

Compound P₅ is itself obtained via nitration of compound P₆ according to Scheme 10:

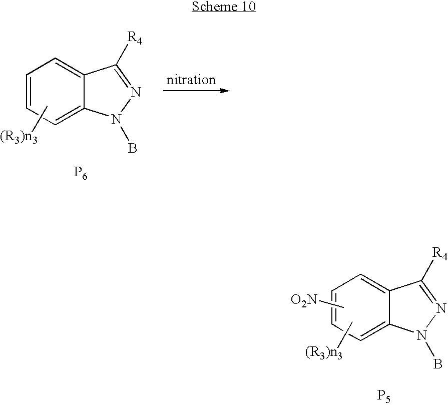

The nitration may be performed according to a method common to those skilled in the art, for example using a nitric acid/sulfuric acid mixture at a temperature of between 20° C. and the boiling point of the reaction medium. For further details regarding the nitration reaction, reference may be made to the following publication: “Nitration: methods and mechanisms, Olah et al., VCH, New-York, 1989”. The conditions of the examples of patent application FR 2 836 914 may also be adapted.

Preparation of Compounds P₅ and P₆

Compounds P₅ and P₆ may be obtained according to different synthetic routes. One of them is a cyclization, respectively, of compounds P₇ and P₈ in the presence of hydrazine, optionally followed by introduction of the protecting group PG₂ (Scheme 11):

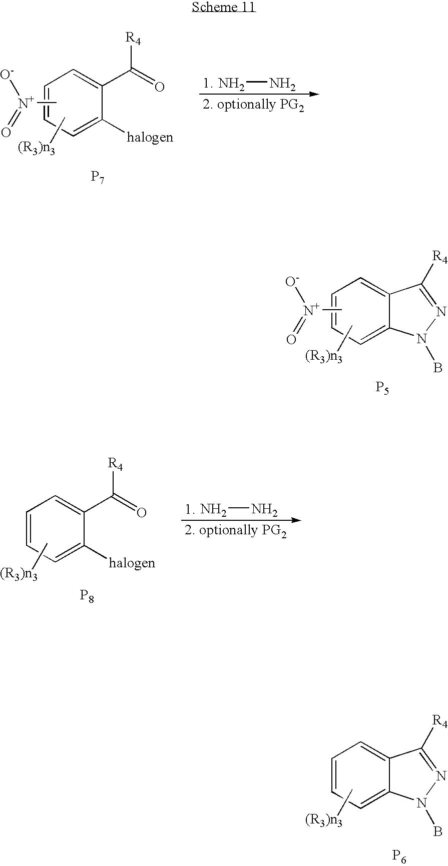

The cyclization reaction is preferably performed in an inert solvent such as an alcohol (for example methanol or ethanol) at a temperature of between 0° C. and the boiling point of the reaction medium. The compounds P₈ may be commercially available or prepared by application or adaptation of the methods described in the following articles: Chem. Pharm. Bull. 1997, 45(9), 1470, Kumazawa, E.; J. Med. Chem. 1991, 34(5), 1545, Bellamy, F. D.; Synth. Commun. 1991, 21(4), 505, Deutsch, J.; J. Het. Chem. 1996, 33(3), 831, Varvarescou, A. or alternatively in WO 93/22287.

Another method for obtaining compounds P₅ or P₆ consists in reacting, respectively, compounds P₉ or P₁₀ with a nitrite RONO (sodium nitrite, tert-butyl nitrite or isoamyl nitrite, for example) in the presence of an acid (for example acetic acid) or an acid anhydride (for example acetic anhydride), preferably at a temperature of between 0° C. and the boiling point of the reaction medium (Scheme 12).

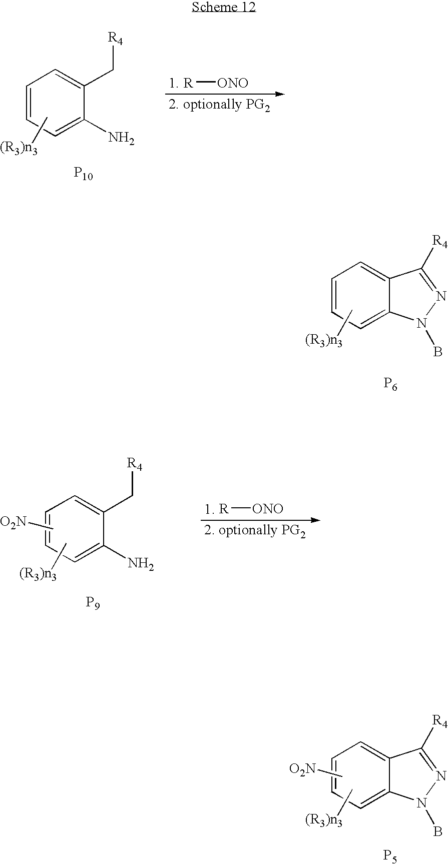

Compounds P₅ and P₆ with R₄═NH₂ may be obtained via cyclization, respectively, of compounds P₁₁ and P₁₂ in the presence of hydrazine (Stocks, M. J. Bioorganic & Medicinal Chemistry Letters 2005, 15(14), 3459-3462; Lukin, K. J. Org. Chem. 2006, 71(21), 8166-8172), optionally followed by introduction of PG₂ (Scheme 13).

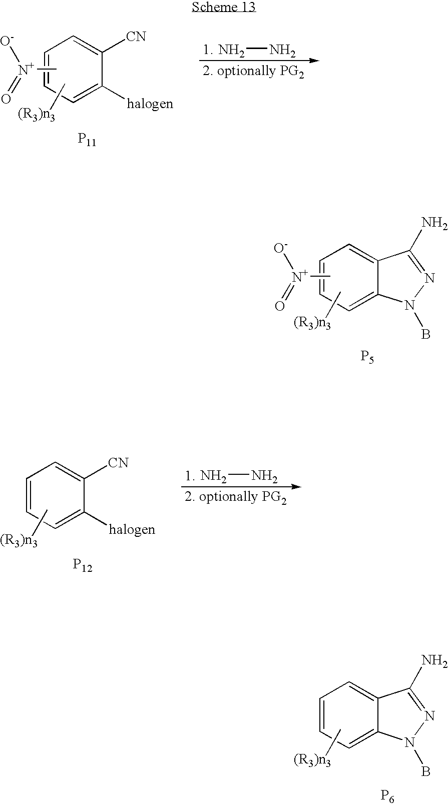

It is understood that for ease of synthesis, and for some of the compounds, R₄ may be introduced before or after coupling, using a precursor of R₄, referenced as R′₄ (Scheme 14). Thus, R′₄ may first be converted into R₄, followed by the coupling (route 1). It is also possible to perform the coupling first, and then to convert R′₄ into R₄ (route 2). In this scheme, C denotes one of the following groups: THN—; Z—O—CO—NT-; XCN— or NO₂—.

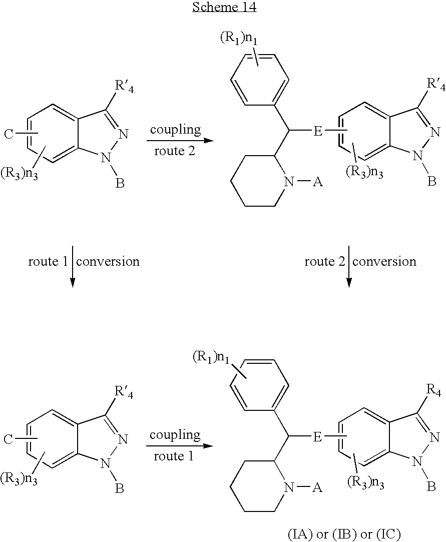

Thus, it is possible to convert R′₄═NH₂ into:
R₄═NRR′: via alkylation using a halide (R)(R′)-Hal. The reaction is performed in the presence of a base at a temperature of between 0° C. and the boiling point of the reaction medium (cf. H. Kawakubo et al., Chem. Pharm. Bull. 1987, 35(6), 2292);
R₄═NHR, R representing a disubstituted alkyl group: via a reaction using an aldehyde or a ketone in the presence of a reducing agent (cf. M. B. Smith and J. March, Wiley Interscience, “Advanced Organic Chemistry”, 5th edition, p. 1185);
R₄═NH—C(═O)—R′: via acylation using an acylating agent allowing the introduction of the group R′CO—; this group may be an acid chloride R′—C(═O)Cl (preferably in the presence of a base such as pyridine, triethylamine or diisopropylethylamine; reaction in an inert solvent such as DMF or THF; cf. G. Daidone et al. “Heterocycles” 1996, 43(11), 2285), an acid anhydride (R′CO)2O (reaction in an inert solvent such as DMF or THF or in the anhydride itself (cf. F. Albericio Synth. Commun. 2001, 41(2), 225; G. Procter Tetrahedron 1995, 51(47), 12837) or an acid R′COOH (cf. M. Bodanszky et al. “Principles of Peptide Synthesis”, Springer-Verlag, New York, 1984, 9-58).

Similarly, R′₄═CHO may be converted into R₄=benzimidazole via the action of o-phenylene-diamines by application of methods that are common to those skilled in the art. And, R₄=alkyl, alkylene, alkynyl, aryl, heteroaryl, heterocycloalkyl, NR—CO—R′, COOR, NRR′, CHO or CONR(OR′) group, may be obtained via reactions using palladium chemistry (cf. A. Suzuki, Pure Appl. Chem. 1991, 63, 419; J. Stille, Angew. Chem. Int. Ed. 1986, 25, 508; R. F. Heck, Org. React. 1982, 27, 345; K. Sonogashira, Synthesis 1977, 777; S. L. Buchwald, Acc. Chem. Rev. 1998, 31, 805) or copper chemistry (Buchwald, Organic Letters 2002, 4(4), 581) starting with the corresponding halo, triflate and mesylate derivatives.

Preparation of an Enantiomerically Enriched Compound According to the Invention

Each coupling described previously may be performed with the enantiomers or diastereoisomers, or mixtures thereof, of compounds P₁ or P₃, optionally followed by a separation step (for example chiral chromatography or recrystallization).

The diastereoisomers (2S, RS) and (2R, RS) of compounds P₁ may also be obtained, on the one hand, by separation of the racemic mixtures, for example by chromatography on a column of silica. They may also be obtained using the process of Scheme 15 starting with an enantiomerically pure aldehyde (1) (R or S).

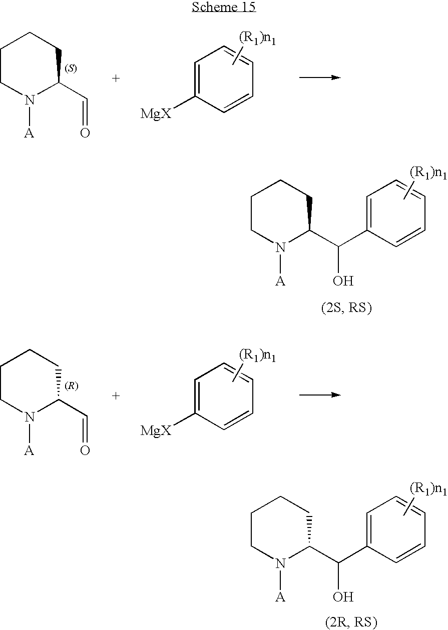

The enantiomers (2S,S), (2S,R), (2R,S) and (2R,R) of compounds P₁ may be obtained via separation of the racemic mixtures or of the diastereoisomers (for example by chromatography on a column of silica) (Scheme 16).

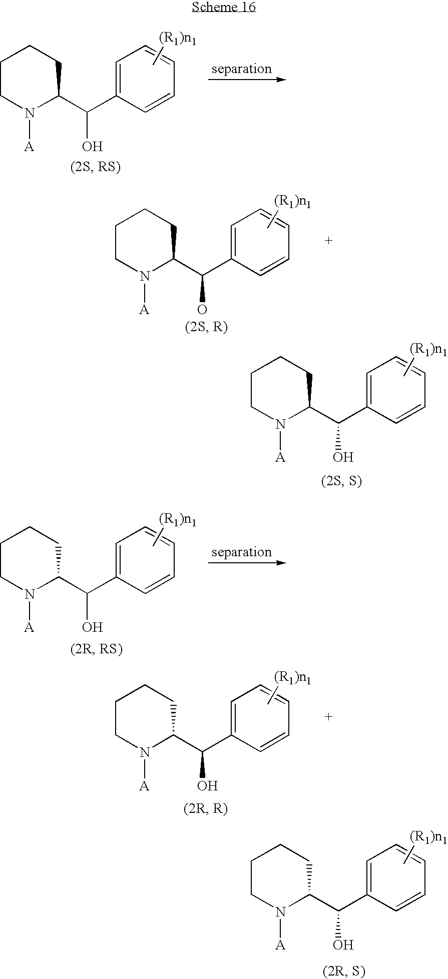

The enantiomers (2S,S) and (2R,R) of compounds P₁ may also be obtained from the diastereoisomers via oxidation of the alcohol function, followed by an enantioselective reduction (Scheme 17):

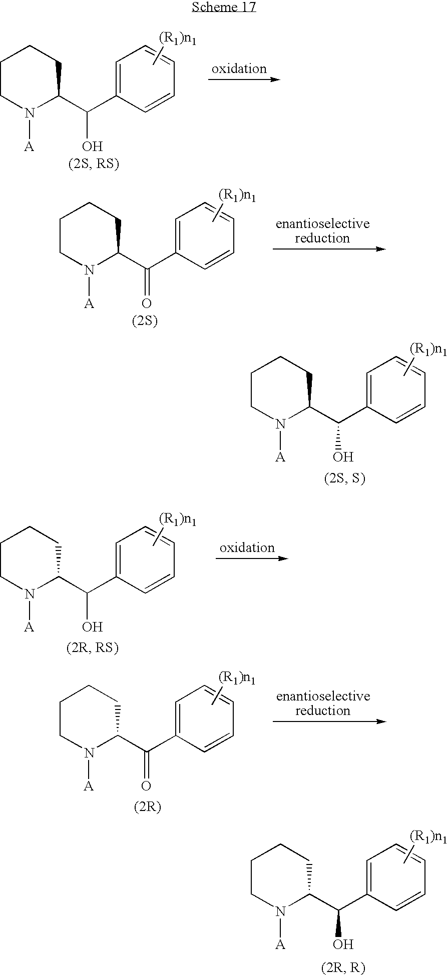

The compounds P₁ of configuration (2S, RS) or (2R, RS) are oxidized to the ketone (2S) or (2R), respectively, according to methods that are common to those skilled in the art, using an oxidizing agent, for instance oxalyl chloride in the presence of dimethyl sulfoxide in a chlorinated solvent such as dichloromethane (DCM) at a temperature of between −70° C. and 20° C. The ketones are then enantioselectively reduced to the alcohols (2S,S) or (2R,R), respectively, via a reducing agent such as K-Selectride® or L-Selectride® (potassium or lithium tri-sec-butylborohydride) in an ether solvent such as THF at a temperature of between −70° C. and 20° C.

All the schemes 1 to 14 apply to the enantiomers and diastereoisomers, and also to mixtures thereof.

### EXAMPLES

The examples that follow illustrate the invention without, however, limiting it.

LC/MS Analyses (1st Method)

The LC/MS analyses were performed using a Waters ZQ model machine connected to an Alliance 2695 machine. The abundance of the products was measured using a Waters 996 PDA diode array detector over a wavelength range of 210-650 nm and a Sedex 85 light-scattering detector. The mass spectra were acquired over a range from 100 to 1000. The data were analysed using the Waters MassLynx software. The separation was performed on a Kromasil C18, 3.5 μm column (50×2.0 mm), eluting with a linear gradient from 0 to 100% of acetonitrile containing 0.05% (v/v) of trifluoroacetic acid (TFA) in water containing 3% acetonitrile (v/v) and 0.05% (v/v) TFA over 13 minutes at a flow rate of 0.5 mL/minute. An isocratic stage of 3 minutes at 100% B followed by equilibration before the next injection at 0% B allows a total analysis time of 20 minutes. The column is at 40° C. The MS spectra were acquired by electrospray-chemical ionization (ESCl+) on the ZQ machine (Waters). The main ions observed are described.

LC/MS Analyses (2nd Method)

The spectra were obtained by LC/MS coupling in electrospray + and − mode (ES+ and ES−) on a ZQ (Waters) machine or a Quattro Premier spectrometer (-Waters). The chromatographic conditions are as follows: ZQ: ZQ X-Bridge C18 2.5 μm 3×50 mm column; flow rate: 1100 μl/minute; gradient: from 5 to 100% of B (CH₃CN) over 5 minutes (A: H₂O+0.1% of formic acid); Quattro Premier: Acquity C18 1.7 μm 2.1×100 mm column; flow rate: 600 μl/minute; gradient: from 5 to 100% of B (MeOH) over 9 minutes (A: H₂O+0.1% of formic acid).

NMR Analyses (1st Method)

Brüker DPX-200 spectrometer (200 MHz), solvent: DMSO-d6 or CDCl₃.

NMR Analyses (2nd Method)

Brüker Avance DX-400 spectrometer (400 MHz), solvent: DMSO-d6 referenced to 2.50 ppm at a temperature of 303 K

### Preparation of Compounds P₁

### Prep 1: tert-Butyl 2-[(3,5-dichlorophenyl)(hydroxy)methyl]piperidine-1-carboxylate

In a three-necked flask equipped with a magnetic stirrer and placed under a nitrogen atmosphere, tert-butyl 2-formylpiperidine-1-carboxylate (4.9 g, 22.98 mmol) is dissolved in tetrahydrofuran (150 mL). (3,5-Dichlorophenyl) bromide (0.5 M solution in THF) (55.1 mL, 27.6 mmol) is added dropwise at −70° C. The mixture is stirred at −70° C. for 5 hours and then hydrolysed by addition of water at 0° C. The medium is diluted with EtOAc, washed with water and with saturated NaCl solution and then dried over Na₂SO₄ and concentrated under vacuum. The residue is purified by chromatography on silica gel eluted with a 9/1 to 4/1 heptane/EtOAc gradient. 2.65 g of tert-butyl 2-[(3,5-dichlorophenyl)(hydroxy)methyl]piperidine-1-carboxylate (50/50 mixture of diastereoisomers) are obtained in the form of an oil. (M+H)⁺=360.5

### Prep 2: tert-Butyl (2R)-2-[(3,4-dichlorophenyl)(hydroxy)methyl]piperidine-1-carboxylate

The compound is prepared according to Preparation 1 starting with tert-butyl (R)-2-formylpiperidine-1-carboxylate and (3,4-dichlorophenyl) bromide. (M+H)⁺=360.6

### Prep 3: tert-Butyl (2S)-2-[(3,4-dichlorophenyl)(hydroxy)methyl]piperidine-1-carboxylate

The compound is prepared according to Preparation 1 starting with tert-butyl (S)-2-formylpiperidine-1-carboxylate and (3,4-dichlorophenyl)magnesium bromide. (M−H+HCO₂H]⁻=404

### Prep 4: tert-Butyl (2R)-2-[(S)-(3-chloro-5-fluorophenyl)(hydroxy)methyl]piperidine-1-carboxylate and tert-butyl (2R)-2-[(R)-(3-chloro-5-fluorophenyl)(hydroxy)methyl]piperidine-1-carboxylate

In a three-necked flask equipped with a magnetic stirrer and placed under a nitrogen atmosphere, tert-butyl (R)-2-formylpiperidine-1-carboxylate (3.7 g, 17.3 mmol) is dissolved in tetrahydrofuran (50 mL). (3-Chloro-5-fluorophenyl) bromide (0.5 M solution in THF) (41.6 mL, 20.8 mmol) is added dropwise at −70° C. The mixture is stirred at −70° C. for 5 hours and then hydrolysed by addition of water at 0° C. The medium is diluted with EtOAc, washed with water and with saturated NaCl solution and then dried over MgSO₄ and concentrated by evaporation under reduced pressure (RP). The residue is purified by chromatography on silica gel eluted with a 6/2 cyclohexane/EtOAc mixture. 1.28 g of tert-butyl (2R)-2-[(S)-(3-chloro-5-fluorophenyl)(hydroxy)methyl]piperidine-1-carboxylate are thus obtained in the form of an oil that crystallizes, and 1.27 g of tert-butyl (2R)-2-[(R)-(3-chloro-5-fluorophenyl)(hydroxy)methyl]piperidine-1-carboxylate are obtained in the form of an oil that crystallizes. tert-Butyl (2R)-2-[(S)-(3-chloro-5-fluorophenyl)(hydroxy)methyl]piperidine-1-carboxylate: (M+Na)⁺=366; [α]_{D}^{20° C.}=+16.4°±0.6 (MeOH) and tert-butyl (2R)-2-[(R)-(3-chloro-5-fluorophenyl)(hydroxy)methyl]piperidine-1-carboxylate: (M+Na)⁺=366; [α]_{D}^{20° C.}=−46.1°±0.9 (MeOH)

### Prep 5: tert-Butyl (2S)-2-[(R)-(3-chloro-5-fluorophenyl)(hydroxy)methyl]piperidine-1-carboxylate and tert-butyl (2S)-2-[(S)-(3-chloro-5-fluorophenyl)(hydroxy)methyl]-piperidine-1-carboxylate

The compounds are obtained according to Preparation 4, replacing the tert-butyl (R)-2-formylpiperidine-1-carboxylate with tert-butyl (S)-2-formylpiperidine-1-carboxylate (19 g, 89 mmol). tert-Butyl (2S)-2-[(R)-(3-chloro-5-fluorophenyl)(hydroxy)methyl]piperidine-1-carboxylate and tert-butyl (2S)-2-[(S)-(3-chloro-5-fluorophenyl)(hydroxy)methyl]piperidine-1-carboxylate are thus obtained. tert-Butyl (2S)-2-[(R)-(3-chloro-5-fluorophenyl)(hydroxy)methyl]piperidine-1-carboxylate: (M−H)⁺=344, m.p. (melting point) ˜110° C.; [α]_{D}^{20° C.}=−8.7°±0.4 (MeOH) and tert-butyl (2S)-2-[(S)-(3-chloro-5-fluorophenyl)(hydroxy)methyl]piperidine-1-carboxylate: (M−H)⁺=344, m.p.=107-109° C., [α]_{D}^{20° C.}=+66.1°±1.4 (MeOH)

### Prep 6: tert-Butyl (2S)-2-[(S)-(3-chloro-5-fluorophenyl)(hydroxy)methyl]piperidine-1-carboxylate

This compound, the preparation of which is described in Preparation 5, may also be obtained in the following manner:

### Prep 6a: tert-Butyl 2(S)-2-(3-chloro-5-fluorobenzoyl)piperidine-1-carboxylate

In a three-necked flask equipped with a magnetic stirrer and placed under a nitrogen atmosphere, oxalyl chloride (3.4 mL, 39.3 mmol) is dissolved in DCM (40 mL) at −70° C. DMSO (4.3 mL) is added dropwise, followed by addition of tert-butyl (S)-2-[(3-chloro-5-fluorophenyl)(hydroxy)methyl]piperidine-1-carboxylate prepared in Preparation 5 (7.5 g, 21.8 mmol) dissolved in DCM (160 mL). The mixture is stirred for 30 minutes and triethylamine is then added (12.8 mL, 91.8 mmol). After stirring for 1 hour 30 minutes between −70° C. and 20° C., water is added. The organic phase is washed with saturated sodium bicarbonate solution, dried over Na₂SO₄ and concentrated under vacuum. tert-Butyl 2(S)-2-(3-chloro-5-fluorobenzoyl)piperidine-1-carboxylate (7.0 g) is obtained in the form of an oil. (M+H)⁺=342.3

### Prep 6b: tert-Butyl (2S)-2-[(S)(3-chloro-5-fluorophenyl)(hydroxy)methyl]piperidine-1-carboxylate

In a three-necked flask equipped with a magnetic stirrer and placed under a nitrogen atmosphere, tert-butyl 2(S)-2-(3-chloro-5-fluorobenzoyl)piperidine-1-carboxylate (7.0 g, 20.5 mmol) is dissolved in THF (150 mL) at −70° C. A 1M solution of L-Selectride in THF is then added dropwise and the reaction medium is stirred for 1 hour. 35 mL of water and then 3 mL of 32% H₂O₂ are added cautiously and successively, while maintaining the temperature <5° C. The reaction medium is then extracted with EtOAc. The organic phase is washed twice with 10% sodium thiosulfate solution and then with water and finally with saturated NaCl solution. It is dried over Na₂SO₄ and concentrated under vacuum. The residue is purified by chromatography on silica gel diluted with a 9/1 and then 5/1 n-heptane/EtOAc mixture. tert-Butyl (2S)-2-[(S)(3-chloro-5-fluorophenyl)(hydroxy)methyl]piperidine-1-carboxylate (5.8 g) is obtained in the form of an oil. (M+H)⁺=344.4

### Prep 7: tert-Butyl (2S)-2-{[3-(ethoxycarbonyl)phenyl](hydroxy)methyl}piperidine-1-carboxylate

Into a three-necked flask equipped with a magnetic stirrer and placed under argon is introduced ethyl 3-iodobenzoate (1.05 mL, 6.33 mmol) dissolved in tetrahydrofuran (10 mL) and the solution is cooled to about −25° C. using an isopropanol-cardice bath. A 1M solution of isopropylmagnesium bromide in THF (6.5 mL, 6.5 mmol) is then added over about 10 minutes, while maintaining the temperature <−25° C. The solution thus obtained is maintained at −30° C. for 1 hour, and a solution of tert-butyl (S)-2-formylpiperidine-1-carboxylate (1.07 g, 5 mmol) in 5 mL of THF is then run in, over 2 minutes and at −25° C., while maintaining the temperature at about −20° C. The mixture is then brought to −70° C. and maintained at this temperature for 2 hours. After cooling to about 0° C., the medium is hydrolysed with saturated aqueous ammonium chloride solution (30 mL). After separating the two phases, the aqueous phase is extracted with twice 20 mL of EtOAc. The combined organic extracts are washed with distilled water (30 mL) and then saturated aqueous NaCl solution (15 mL), dried over MgSO₄, filtered and concentrated to dryness under RP. The isolated yellow oil is chromatographed on 100 g of silica gel 60, particle size 15-40 μm, contained in a column 3 cm in diameter, eluting with a 3/1 v/v cyclohexane/EtOAc mixture, under a positive pressure of 0.6 bar of argon. Evaporation of the fractions gives 1.22 g of tert-butyl (2S)-2-{[3-(ethoxycarbonyl)phenyl](hydroxy)methyl}piperidine-1-carboxylate in the form of a colourless oil. (M+H)⁺=364. ¹H NMR (DMSO, 400 MHz): 50%-50% mixture of isomers, δ (ppm) from 0.98 to 1.83 (m, 17.5H); 2.13 (m, 0.5H); 2.92 (m, 1H); 3.90 (m, 1H); 4.10 (broad m, 1H); 4.31 (q, J=7.5 Hz, 2H); 4.89 (m, 1H); 5.36 (broad s, 0.5H); 5.61 (broad s, 0.5H); 7.40 (t, J=7.5 Hz, 0.5H); 7.48 (t, J=7.5 Hz, 0.5H); 7.53 (broad d, J=7.5 Hz, 0.5H); 7.60 (broad d, J=7.5 Hz, 0.5H); 7.81 (broad d, J=7.5 Hz, 0.5H); 7.86 (td, J=1.5 and 7.5 Hz, 0.5H); 8.00 (broad s, 1H).

### Prep 10: tert-Butyl (2S)-2-[(S)-(3-bromophenyl)hydroxymethyl]piperidine-1-carboxylate

### Prep 10a: tert-Butyl (S)-2-(3-bromobenzoyl)piperidine-1-carboxylate

The compound may be prepared as described in WO2008/018639 on page 105.

### Prep 10b: tert-Butyl (2S)-2-[(S)-(3-bromophenyl)hydroxymethyl]piperidine-1-carboxylate

The compound is prepared according to Preparation 6b starting with tert-butyl (S)-2-(3-bromobenzoyl)piperidine-1-carboxylate. (M+Na)⁺=392.

### Prep 11: tert-Butyl (2S)-2-[(S)-(3-trifluoromethylphenyl)hydroxymethyl]piperidine-1-carboxylate

### Prep 11a: tert-Butyl (2S)-2-(N-methoxy-N-methylcarbamoyl)piperidine-1-carboxylate

The compound may be prepared as described in S. T. Tong et al. Tetrahedron Letters 2006, 47(29), 5017-5020.

### Prep 11b: tert-Butyl (S)-2-(3-trifluoromethylbenzoyl)piperidine-1-carboxylate

In a first three-necked flask equipped with a magnetic stirrer and placed under a nitrogen atmosphere, a solution of 3-bromo-5-trifluoromethylbenzene (7 g, 31.2 mmol) and 60 mL of diethyl ether is cooled to a temperature in the region of −78° C. A solution of n-butyllithium (1.6 M solution in THF) (21.4 mL, 34.3 mmol) is then added dropwise and stirring is continued at −78° C. for 30 minutes. Into a second three-necked flask equipped with a magnetic stirrer and placed under nitrogen are introduced tert-butyl (2S)-2-(N-methoxy-N-methylcarbamoyl)piperidine-1-carboxylate (8.5 g, 31.2 mmol) and 125 mL of diethyl ether. After cooling to −78° C., the solution obtained previously in the first three-necked flask is introduced dropwise. The reaction medium is then maintained at −75° C. for 2 hours, and the temperature is allowed to rise to 0° C., followed by addition of 130 mL of saturated aqueous NaHCO₃ solution. The organic phase is then washed with water, dried over MgSO₄, filtered and concentrated by evaporation under RP. The oil thus obtained is purified by chromatography on silica gel (eluent: 8/2 cyclohexane/EtOAc). 5.3 g of tert-butyl (S)-2-(3-trifluoromethylbenzoyl)piperidine-1-carboxylate are thus obtained in the form of a yellow oil. (M+H−tBuOCO)⁺=258.

### Prep 11c: tert-Butyl (2S)-2-[(S)-(3-trifluoromethylphenyl)hydroxymethyl]piperidine-1-carboxylate

The compound is prepared according to Preparation 6b starting with tert-butyl (S)-2-(3-trifluoromethylbenzoyl)piperidine-1-carboxylate. (M+H)⁺=360.

### Prep 12: tert-Butyl (2S)-2-[(S)-(3-iodophenyl)(hydroxy)methyl]piperidine-1-carboxylate

### Prep 12a: tert-Butyl (S)-2-(3-iodobenzoyl)piperidine-1-carboxylate

The compound is prepared according to Preparation 11b starting with diiodobenzene (5.43 g, 0.0165 mol) and tert-butyl (2S)-2-(N-methoxy-N-methylcarbamoyl)piperidine-1-carboxylate (4.49 g, 0.0165 mol). 2.18 g of tert-butyl (S)-2-(3-iodobenzoyl)piperidine-1-carboxylate are thus obtained in the form of a yellow oil that crystallizes. (M+H−tBuOCO)⁺=316.

### Prep 12b: tert-Butyl (2S)-2-[(S)-(3-iodophenyl)(hydroxy)methyl]piperidine-1-carboxylate

The compound is prepared according to Preparation 6b starting with tert-butyl (S)-2-(3-iodobenzoyl)piperidine-1-carboxylate. (M+H)⁺=418.

### Preparation of Compounds P₃

### Prep 8: (S)-1-[(2S)-(1-Allylpiperid-2-yl)-1-(3,4-dichlorophenyl)methanamine

### Prep 8a: (3,4-Dichlorophenyl)[(2S)-piperid-2-yl)]ethanol

In a three-necked flask equipped with a magnetic stirrer and placed under a nitrogen atmosphere, tert-butyl (2S)-2-[(3,4-dichlorophenyl)(hydroxy)methyl]piperidine-1-carboxylate (6.5 g, 18 mmol) is dissolved in dioxane (20 mL). A 4N solution of HCl in dioxane (55 mL, 220 mmol) is added and the mixture is stirred at room temperature (RT) for 4 hours. The reaction medium is concentrated under vacuum to give 5.4 g of (3,4-dichlorophenyl)[(2S)-piperid-2-yl)]ethanol hydrochloride.

### Prep 8b: (S)-[(2S)-1-Allylpiperid-2-yl](3,4-dichlorophenyl)methanol and (R)-[(2S)-1-allylpiperid-2-yl](3,4-dichlorophenyl)methanol

In a three-necked flask equipped with a magnetic stirrer and placed under a nitrogen atmosphere, (3,4-dichlorophenyl)(piperid-2-(S)-yl)methanol hydrochloride (5.3 g, 17.9 mmol) is dissolved in acetonitrile (180 mL). Potassium carbonate (6.17 g, 44.7 mmol) and allyl bromide (1.85 mL, 21.4 mmol) are successively added. The mixture is stirred for 18 hours and the solvent is then removed by evaporation. The residue is taken up in DCM and washed with water. The organic phase is dried over Na₂SO₄ and concentrated under vacuum. The residue is purified by chromatography on silica gel diluted with a 99/1/0.1 and then 96/4/0.4 DCM/MeOH/NH₄OH mixture. 2.2 g of (S)-[(2S)-1-allylpiperid-2-yl](3,4-dichlorophenyl)-methanol and 2.2 g (R)-[(2S)-1-allylpiperid-2-yl](3,4-dichlorophenyl)methanol are obtained. (M+H)⁺=300.

### Prep 8c: (2S)-1-Allyl-2-[(R)-chloro(3,4-dichlorophenyl)methyl]piperidine

In a three-necked flask equipped with a magnetic stirrer and placed under a nitrogen atmosphere, (S)-[(2S)-1-allylpiperid-2-yl](3,4-dichlorophenyl)methanol (1 g, 3.3 mmol) and potassium carbonate (1 g, 7.3 mmol) are dissolved in DCM (42 mL). Mesyl chloride (0.57 mL, 7.3 mmol) is added dropwise at 4° C. The mixture is stirred for 1 hour 30 minutes at 4° C. and a further amount of potassium carbonate (0.5 g, 3.7 mmol) and mesyl chloride (0.28 mL, 3.7 mmol) is added at 4° C. The medium is stirred for 18 hours at RT and the solvent is evaporated off under vacuum. The residue is taken up in DCM and washed with water. The organic phase is dried over Na₂SO₄ and concentrated under vacuum. 1.2 g of (2S)-1-allyl-2-[(R)-chloro(3,4-dichlorophenyl)methyl]piperidine are obtained. (M+H)⁺=320.

### Prep 8d: (S)-1-[(2S)-1-Allylpiperid-2-yl]-1-(3,4-dichlorophenyl)methanamine

Into a round-bottomed flask equipped with a magnetic stirrer are introduced (2S)-1-allyl-2-[(R)-chloro(3,4-dichlorophenyl)methyl]piperidine (1 g, 3.1 mmol) and a 2N solution of NH₃ in methanol (30 mL, 60 mmol). The reaction medium is then stirred for 18 hours at RT and the solvent is then evaporated off under vacuum. The residue is purified by chromatography on silica gel diluted with a 98/2/0.2 and then 97/3/0.3 DCM/NaOH/NH₄OH mixture. 0.45 g of (S)-1-[(2S)-1-allylpiperid-2-yl]-1-(3,4-dichlorophenyl)methanamine is obtained. (M+H)⁺=299.

### Prep 9: (S)-1-[(2S)-1-Allylpiperid-2-yl]-1-(3-chloro-5-fluorophenyl)methanamine

### Prep 9a: (S)(3-Chloro-5-fluorophenyl)[(2S)piperid-2-(S)-yl]methanol

This compound is prepared according to the procedure described in Prep. 8a starting with tert-butyl (2S)-2-[(S)(3-chloro-5-fluorophenyl)(hydroxy)methyl]piperidine-1-carboxylate described in Preparation 6b (5.8 g, 16.9 mmol). 5.1 g of (S)(3-chloro-5-fluorophenyl)[(2S)piperid-2-(S)-yl]methanol are obtained.

### Prep 9b: (S)-[(2S)-1-Allylpiperid-2-yl](3-chloro-5-fluorophenyl)methanol

This compound is prepared according to the procedure described in Preparation 8b starting with (S)(3-chloro-5-fluorophenyl)[(2S)piperid-2-(S)-yl]methanol (4.6 g, 16.4 mmol). 4.6 g of (S)-[(2S)-1-allylpiperid-2-yl](3-chloro-5-fluorophenyl)methanol are obtained. (M+H)⁺=284

### Prep 9c: (2S)-1-Allyl-2-[(R)-chloro(3-chloro-5-fluorophenyl)methyl]piperidine

This compound is prepared according to the procedure described in Prep. 8c starting with (S)-[(2S)-1-allylpiperid-2-yl](3-chloro-5-fluorophenyl)methanol (4.1 g, 14.5 mmol). (M+H)⁺=302

### Prep 9d: 1-[(S)-[(2S)-1-Allylpiperid-2-yl](3-chloro-5-fluorophenyl)methylamine

This compound is prepared according to the procedure described in Prep. 8d starting with (2S)-1-allyl-2-[(R)-chloro(3-chloro-5-fluorophenyl)methyl]piperidine (4.6 g, 15.2 mmol). 2.5 g of 1-[(S)-[(2S)-1-allylpiperid-2-yl](3-chloro-5-fluorophenyl)methylamine are obtained. (M+H)⁺=283

The following amines were obtained according to the process described in Preparations 8 or 9, using the appropriate alcohols: (S)-1-[(2S)-1-allylpiperid-2-yl]-1-phenylmethanamine; (R)-1-[(2R)-1-allylpiperid-2-yl]-1-phenylmethanamine; 1-[1-allylpiperid-2-yl]-1-(3-chloro-5-fluorophenyl)methanamine; (R)-1-[(2R)-1-allylpiperid-2-yl]-1-(3,4-dichlorophenyl)methanamine; (S)-1-[(2S)-1-allylpiperid-2-yl]-1-(3,5-dichlorophenyl)-methanamine; (S)-1-[(2S)-1-allylpiperid-2-yl]-1-(3,4-dichlorophenyl)methanamine; 1-[(1-methylpiperid-2-yl]-1-phenylmethylamine((S,2S),(R,2R)): this compound may be prepared as described in FR 2 842 805.

### Preparation of the Compounds According to the Invention

### Ex 1: 1-(1H-Indazol-5-yl)-3-[(1-methylpiperid-2-yl)(phenyl)methyl]urea((S,2S),(R,2R))

### Ex 1a: 5-Nitro-1-{[2-(trimethylsilyl)ethoxy]methyl}indazole

In a round-bottomed flask equipped with a magnetic bar, 5-nitroindazole (2 g, 12.26 mmol) is dissolved in 60 mL of DCM. Trimethylsilylethoxymethyl chloride (4.34 mL, 24.52 mmol) and, dropwise, diisopropylethylamine (4.27 mL, 24.52 mmol) are added at 0° C. After stirring for 3 hours at RT, water is added and the medium is extracted with DCM. After drying the organic phase over Na₂SO₄ and evaporating off the solvents, the residue is purified by chromatography on silica gel (eluent: 97/3 cyclohexane/EtOAc) to give 1.65 g of 5-nitro-1-{[2-(trimethylsilyl)ethoxy]methyl}indazole. (M+H)⁺=294.

### Ex 1b: 1-{[2-(Trimethylsilyl)ethoxy]methyl}indazole-5-amine

In a Parr flask, 5-nitro-1-{[2-(trimethylsilyl)ethoxy]methyl}indazole (0.52 g, 1.77 mol) is dissolved in 80 mL of MeOH and palladium-on-charcoal (0.05 g, 0.02 mmol) is added under N₂. The mixture is stirred under 3 atm H₂ for 3 hours. After filtering off the catalyst and evaporating off the MeOH, 0.46 g of 1-{[2-(trimethylsilyl)ethoxy]methyl}indazole-5-amine is recovered. (M+H)⁺=264.

### Ex 1c: 1-[1-{[2-(Trimethylsilyl)ethoxy]methyl}indazol-5-yl]3-[(1-methylpiperid-2-yl)(phenyl)methyl]urea((S,2S),(R,2R))

In a round-bottomed flask equipped with a magnetic bar, 1-{[2-(trimethylsilyl)ethoxy]methyl}indazole-5-amine (0.3 g, 1.14 mmol) is dissolved in 20 mL of DCM. Triethylamine (0.16 mL, 1.14) is added. Next, triphosgene (0.113 g, 0.38 mmol) is dissolved in 30 mL of DCM and added dropwise to the reaction medium. After reaction for 5 hours, the reaction medium is poured into water and extracted with DCM. The organic phase is washed with saturated sodium hydrogen carbonate solution. After drying, filtering and evaporating the organic phase, 0.3 g of an oil is recovered, which is dissolved in 100 mL of diethyl ether in a round-bottomed flask equipped with a magnetic bar. 1-[(1-Methylpiperid-2-yl]-1-phenylmethylamine((S,2S),(R,2R)) (0.21 g, 1.03 mmol) dissolved in 10 mL of diethyl ether is added to the reaction medium. After stirring for 3 hours, the organic solvents are evaporated off and the residue is purified by chromatography on silica gel (eluent: 96/4 DCM/MeOH) to give 0.11 g of 1-[1-{[2-(trimethylsilyl)ethoxy]methyl}indazol-5-yl]3-[(1-methylpiperid-2-yl)(phenyl)methyl]urea((S,2S),(R,2R)). (M+H)⁺=494.

### Ex 1d: 1-(1H-Indazol-5-yl)-3-[(1-methylpiperid-2-yl)(phenyl)methyl]urea((S,2S),(R,2R))

In a round-bottomed flask equipped with a magnetic bar, 1-[1-{[2-(trimethylsilyl)ethoxy]methyl}indazol-5-yl]3-[(1-methylpiperid-2-yl)(phenyl)methyl]urea((S,2S),(R,2R)) (0.094 g, 0.19 mmol) is dissolved in 10 mL of DCM. 4N HCl in dioxane (10 mL, 96.3 mmol) is added. After stirring overnight, the solvents are evaporated off and the residue is partitioned between a sodium hydroxide solution and DCM. The organic phase is evaporated and the residue is purified by chromatography on silica gel (eluent: 90/10/1 DCM/MeOH/aqueous ammonia) to give 0.018 g of 1-(1H-indazol-6-yl)-3-[(1-methylpiperid-2-yl)(phenyl)methyl]urea((S,2S),(R,2R)). The product obtained is treated with a molar excess of fumaric acid in ethanol. The fumaric acid salt crystallizes after the addition of diisopropyl ether. (M+H)⁺=480, m.p.=215° C. ¹H NMR (DMSO, 200 MHz): δ (ppm) 8.79 (s, 1H), 7.87 (d, J=1 Hz, 1H), 7.81 (d, J=1 Hz, 1H), 7.41-7.14 (m, 8H), 6.76 (d, J=6.8 Hz, 1H), 6.56 (s, 2H), 4.84 (t, J=6.8 Hz, 1H), 2.87 (m, 1H), 2.25 (s, 3H), 1.74-1.15 (m, 6H).

### Ex 2: 1-(1H-Indazol-6-yl)-3-[(1-methylpiperid-2-yl)(phenyl)methyl]urea((S,2S),(R,2R))

### Ex 2a: 6-Nitro-2-{[2-(trimethylsilyl)ethoxy]methyl}indazole

In a round-bottomed flask equipped with a magnetic bar, 6-nitroindazole (2 g, 12.26 mmol) is dissolved in 60 mL of DCM. Trimethylsilylethoxymethyl chloride (4.34 mL, 24.52 mmol) and, dropwise, diisopropylethylamine (4.27 mL, 24.52 mmol) are added at 0° C. After stirring for 4 hours at RT, water is added and the medium is extracted with DCM. After drying the organic phase over Na₂SO₄ and evaporating off the solvents, the residue is purified by chromatography on silica gel (eluent: 97/3 cyclohexane/EtOAc) to give 0.465 g of 6-nitro-1-{[2-(trimethylsilyl)ethoxy]methyl}indazole. (M+H)⁺=294.

### Ex 2b: 2-{[2-(Trimethylsilyl)ethoxy]methyl}indazole-6-amine

In a Parr flask, 6-nitro-2-{[2-(trimethylsilyl)ethoxy]methyl}indazole (1.1 g, 3.75 mmol) is dissolved in 60 mL of MeOH and palladium-on-charcoal (0.1 g, 0.94 mmol) is added under N₂. The reaction medium is stirred under 3 atm of hydrogen for 1 hour. After filtering off the catalyst and evaporating off the ethanol, 0.85 g of 2-{[2-(trimethylsilyl)ethoxy]methyl}indazole-6-amine is obtained. (M+H)⁺=264.

### Ex 2c: 1-[2-{[2-(Trimethylsilyl)ethoxy]methyl}indazol-6-yl]3-[(1-methyl piperid-2-yl)(phenyl)methyl]urea((S,2S),(R,2R))

In a three-necked flask equipped with a magnetic bar, 2-{[2-(trimethylsilyl)ethoxy]methyl}indazole-6-amine (0.12 g, 0.46 mmol) is dissolved in 10 mL of DCM. Triethylamine (0.08 mL, 0.57 mmol) and triphosgene (0.05 g, 0.57 mmol) are successively added. After stirring for 5 hours at RT, 1-[(1-methylpiperid-2-yl]-1-phenylmethylamine((S,2S),(R,2R)) (0.180 g, 0.88 mmol) dissolved in 10 mL of DCM is added. After stirring overnight, the reaction medium is taken up in water and extracted with DCM. The organic phase is dried and evaporated. The residue is purified by chromatography on silica gel (eluent: 95/5 DCM/MeOH) to give 0.094 g of 1-[2-{[2-(trimethylsilyl)ethoxy]methyl}indazol-6-yl]3-[(1-methylpiperid-2-yl)(phenyl)methyl]urea((S,2S),(R,2R)). (M+H)⁺=494.

### Ex 2d: 1-(1H-Indazol-6-yl)-3-[(1-methylpiperid-2-yl)(phenyl)methyl]urea((S,2S),(R,2R))

In a round-bottomed flask equipped with a magnetic bar, 1-[2-{[2-(trimethylsilyl)ethoxy]methyl}indazol-6-yl]3-[(1-methylpiperid-2-yl)(phenyl)methyl]urea((S,2S),(R,2R)) (0.094 g, 0.19 mmol) is dissolved in 10 mL of DCM. 4N HCl in dioxane (1.9 mL, 7.62 mmol) is added. After stirring overnight, the solvents are evaporated off and the residue is partitioned between a solution of NaOH and DCM. The organic phase is evaporated and the residue is purified by chromatography on silica gel (eluent: 90/10 DCM/MeOH) to give 0.018 g of 1-(1H-indazol-6-yl)-3-[(1-methylpiperid-2-yl)(phenyl)methyl]urea((S,2S),(R,2R)). The product obtained is treated with a molar excess of fumaric acid in ethanol. The fumaric acid salt crystallizes after the addition of diisopropyl ether. (M+H)⁺=480, m.p.=210° C. ¹H NMR (DMSO, 200 MHz): δ (ppm) 9.03 (s, 0.8H), 7.85 (s, 2H), 7.54 (s, 1H), 7.37-7.14 (m, 5H), 6.95-6.75 (m, 2H), 6.58 (s, 1.8H), 4.84 (m, 1H), 2.87 (m, 1H), 2.65 (m, 1H), 2.26 (s, 3H), 1.74-1.14 (m, 6H).

### Ex 3: 1-{(S)-(3-Chloro-5-fluorophenyl)[(2S)-piperid-2-yl]methyl}-3-(1H-indazol-5-yl)urea

### Ex 3a: 1-[(S)-[(2S)-1-Allylpiperid-2-yl](3-chloro-5-fluorophenyl)methyl]-3-(1-{[2-trimethylsilyl)ethoxy]methyl}-1H-indazol-5-yl)urea

In a three-necked flask equipped with a magnetic stirrer and placed under a nitrogen atmosphere, 1-{[2-(trimethylsilyl)ethoxy]methyl}-1H-indazol-5-amine (0.75 g, 2.9 mmol) is dissolved in DCM (20 mL). Triethylamine (0.51 mL, 3.7 mmol) and triphosgene (0.28 g, 0.9 mmol) are successively added at 4° C. The reaction medium is stirred for 3 hours at RT. 1-[(S)-[(2S)-1-Allylpiperid-2-yl](3-chloro-5-fluorophenyl)methylamine (0.8 g, 2.8 mmol) dissolved in DCM (10 mL) is then added at 4° C. The reaction medium is stirred for 18 hours at RT. It is then washed with saturated sodium hydrogen carbonate solution, dried over Na₂SO₄ and concentrated under vacuum. The residue is purified by chromatography on silica gel diluted with a DCM/MeOH/NH₄OH mixture (98/2/0.2 to 96/4/0.4). 0.85 g of 1-[(S)-[(2S)-1-allylpiperid-2-yl](3-chloro-5-fluorophenyl)methyl]-3-(1-{[2-trimethyldilyl)ethoxy]methyl}-1H-indazol-5-yl)urea is obtained. (M+H)⁺=572

### Ex 3b: 1-{(S)-(3-Chloro-5-fluorophenyl)[(2S)-piperid-2-yl]methyl}-3-(1-{[2-trimethylsilyl)-ethoxy]methyl}-1H-indazol-5-yl)urea

In a three-necked flask equipped with a magnetic stirrer, 1,3-dimethylbarbituric acid (0.7 g, 4.5 mmol) and Pd(PPh₃)₄ (0.17 g, 0.15 mmol) are dissolved in DCM (10 mL). The mixture is brought to reflux, 1-[(S)-[(2S)-1-allylpiperid-2-yl](3-chloro-5-fluorophenyl)methyl]-3-(1-{[2-trimethyldilyl)ethoxy]methyl}-1H-indazol-5-yl)urea dissolved in DCM (5 mL) is added, and the resulting mixture is maintained at reflux for 5 hours. After cooling to RT, the medium is washed with saturated sodium hydrogen carbonate solution, dried over Na₂SO₄ and concentrated under vacuum. The residue is purified by chromatography on silica gel diluted with a DCM/MeOH/NH₄OH mixture (98/2/0.2 to 97/3/0.3). 0.67 g of 1-{(S)-(3-chloro-5-fluorophenyl)[(2S)-piperid-2-yl]methyl}-3-(1-{[2-trimethylsilyl)ethoxy]methyl}-1H-indazol-5-yl)urea is obtained. (M+H)⁺=532

### Ex 3c: 1-{(S)-(3-Chloro-5-fluorophenyl)[(2S)-piperid-2-yl]methyl}-3-(1H-indazol-5-yl)urea

Into a round-bottomed flask equipped with a magnetic stirrer and placed under a nitrogen atmosphere is introduced 1-{(S)-(3-chloro-5-fluorophenyl)[(2S)-piperid-2-yl]methyl}-3-(1-{[2-trimethylsilyl)ethoxy]methyl}-1H-indazol-5-yl)urea (0.25 g, 0.5 mmol) in dioxane (5 mL). A 4N solution of HCl in dioxane (5 mL, 20 mmol) and two drops of water are then added. The reaction mixture is stirred for 24 hours at RT and concentrated under vacuum. The residue is purified by chromatography on silica gel diluted with a DCM/MeOH/NH₄OH mixture (99/1/0.1 to 96/4/0.4). 0.1 g of oil is obtained, which is treated with a 0.2N solution of HCl in ethyl ether. After filtration, the 1-{(S)-(3-chloro-5-fluorophenyl)[(2S)-piperid-2-yl]methyl}-3-(1H-indazol-5-yl)urea is obtained in the form of the hydrochloride. (M+H)⁺=401, m.p.=208° C., [α]_{D}^{20° C.}=+38.9° (DMSO) ¹H NMR (DMSO, 200 MHz): δ (ppm) 8.74 (s, 1H), 8.65 (m, 2H), 7.90 (d, J=1 Hz, 1H), 7.80 (dd, J=0.8, 1.8 Hz, 1H), 7.45-7.18 (m, 6H), 4.91 (t, J=8.9 Hz, 1H), 3.28 (m, 1H), 2.81 (m, 1H), 1.78-1.22 (m, 6H).

Examples 4-10 were prepared according to Example 3.

### Ex 4: 1-{(S)-(3,4-Dichlorophenyl)[(2S)-piperid-2-yl]methyl}-3-(1H-indazol-5-yl)urea

(M+H)⁺=418, m.p.=216° C. ¹H NMR (DMSO, 200 MHz): δ (ppm) 12.84 (s, 1H), 8.75-8.51 (m, 3H), 7.90 (d, J=0.8 Hz, 1H), 7.79 (d, J=1.4 Hz, 1H), 7.70-7.63 (m, 2H), 7.37 (m, 2H), 7.21 (m, 2H), 4.89 (t, J=9.1 Hz, 1H), 3.57-3.18 (m, 2H), 2.80 (m, 1H), 1.82-1.19 (m, 6H).

### Ex 5: 1-[(3-Chloro-5-fluorophenyl)(piperid-2-yl)methyl]-3-(1H-indazol-5-yl)urea

(M+H)⁺=402, m.p.=185° C. ¹H NMR (CDCl₃, 400 MHz): δ (ppm) 9.02-8.30 (m, 5H), 7.96-7.78 (m, 3H), 7.56-7.14 (m, 10H), 5.31 (dd, J=9.7, 4.2 Hz, 1H), 4.92 (t, J=9.3 Hz, 1H), 3.01-2.74 (m, 1H), 1.89-1.13 (m, 12H).

### Ex 6: 1-{(R)-(3,4-Dichlorophenyl)[(2R)-piperid-2-yl]methyl}-3-(1H-indazol-5-yl)urea

(M+H)⁺=402, m.p.=220° C. ¹H NMR (DMSO, 200 MHz): δ (ppm) 12.84 (s, 0.6H), 8.81-8.46 (m, 2H), 7.96-7.11 (m, 9H), 4.89 (t, J=9.2 Hz, 1H), 3.57-2.65 (m, 3H), 1.82-1.15 (m, 6H).

### Ex 7: 1-{(S)-(3,5-Dichlorophenyl)[(2S)-piperid-2-yl]methyl}-3-(1H-indazol-5-yl)urea

(M+H)⁺=418. ¹H NMR (DMSO, 200 MHz): δ (ppm) 12.84 (s, 1H), 8.67-8.53 (m, 2H), 7.91 (s, 1H), 7.80 (m, 1H), 7.59 (m, 1H), 7.47 (d, J=1.9 Hz, 2H), 7.43-7.07 (m, 3H), 4.89 (m, 1H), 3.64-2.65 (m, 3H), 1.82-1.23 (m, 6H).

### Ex 8: 1-{(S)-(Phenyl)[(2S)-piperid-2-yl]methyl}-3-(1H-indazol-5-yl)urea

(M+H)⁺=350, m.p.=195° C., [α]_{D}^{20° C.}=+36.1° (DMSO). ¹H NMR (DMSO, 200 MHz): δ (ppm) 9.10 (s, 1H), 7.87 (d, J=1 Hz, 1H), 7.81 (d, J=1 Hz, 1H), 7.51 (m, 1H), 7.41-7.17 (m, 7H), 6.53 (s, 1.7H), 4.75 (t, J=8.1 Hz, 1H), 3.26-2.38 (m, 3H), 1.78-1.14 (m, 6H).

### Ex 9: 1-{(R)-(Phenyl)[(2R)-piperid-2-yl]methyl}-3-(1H-indazol-5-yl)urea

(M+H)⁺=350, m.p.=147-149° C., [α]_{D}^{20° C.}=−67.7° (DMSO). ¹H NMR (DMSO, 200 MHz): δ (ppm) 9.16 (s, 1H), 7.87 (d, J=1 Hz, 1H), 7.82 (m, 1H), 7.64 (d, J=8.4 Hz, 1H), 7.42-7.18 (m, 8H), 6.53 (s, 2.3H), 4.76 (t, J=8.2 Hz, 1H), 3.25-2.52 (m, 3H), 1.74-1.17 (m, 6H).

### Ex 10: 1-{(S)-(Phenyl)[(2S)-1-allylpiperid-2-yl]methyl}-3-(1H-indazol-5-yl)urea

(M+H)⁺=390, m.p.=195° C. ¹H NMR (DMSO, 200 MHz): δ (ppm) 12.80 (m, 1H), 8.75 (s, 1H), 7.86 (d, J=1 Hz, 1H), 7.79 (d, J=1 Hz, 1H), 7.43-7.08 (m, 7H), 6.63 (d, J=6.2 Hz, 1H), 6.58 (s, 2H), 5.80 (m, 1H), 5.09 (m, 2H), 4.85 (t, J=6.7 Hz, 1H), 3.47-2.30 (m, 5H), 1.77-1.07 (m, 6H).

### Ex 11: 1-[(3-Chloro-5-fluorophenyl)(1-methylpiperid-2-yl)methyl]-3-(1H-indazol-5-yl)urea

### Ex 11a: 1-{(S)-(3-Chloro-5-fluorophenyl)[(2S)-1-methyl piperid-2-yl]methyl}-3-(1-{[2-trimethylsilyl)ethoxy]methyl}-1H-indazol-5-yl)urea

Into a round-bottomed flask equipped with a magnetic stirrer and placed under a nitrogen atmosphere, 1-{(S)-(3-chloro-5-fluorophenyl)[(2S)-piperid-2-yl]methyl}-3-(1-{[2-trimethylsilyl)ethoxy]methyl}-1H-indazol-5-yl)urea (Ex. 3b) (0.3 g, 0.56 mmol) is dissolved in MeOH (5 mL). 37% formaldehyde (0.21 mL, 2.82 mmol), sodium cyanoborohydride (0.035 g, 0.556 mmol) and acetic acid (0.03 mL, 0.56 mmol) are successively added. The reaction mixture is stirred for 30 hours and then concentrated under vacuum. The residue is purified by chromatography on silica gel diluted with a DCM/MeOH/NH₄OH mixture (98/2/0.2 to 97/3/0.3). 0.21 g of 1-{(S)-(3-chloro-5-fluorophenyl)[(2S)-1-methylpiperid-2-yl]methyl}-3-(1-{[2-trimethylsilyl)ethoxy]methyl}-1H-indazol-5-yl)urea is obtained. (M+H)⁺=546

### Ex 11b: 1-[(3-Chloro-5-fluorophenyl)(1-methylpiperid-2-yl)methyl]-3-(1H-indazol-5-yl)urea

The compound of Preparation 6b (0.21 g, 0.38 mmol) is treated with a 4N solution of HCl in dioxane (5 mL, 20 mmol) under the conditions described in Ex. 3c. After purification by chromatography on silica gel diluted with a DCM/MeOH/NH₄OH mixture (98/2/0.2 to 96/4/0.4), 0.09 g of 1-[(3-chloro-5-fluorophenyl)(1-methylpiperid-2-yl)methyl]-3-(1H-indazol-5-yl)urea is obtained. The hydrochloride is then prepared after treatment with 0.2N HCl dissolved in ethyl ether and trituration from ethyl ether. (M+H)⁺=416, m.p.=192° C. ¹H NMR (DMSO, 200 MHz): δ (ppm) 9.68 (m, 0.8H), 9.36 (m, 0.3H), 8.94 (m, 1H), 7.91 (d, J=1 Hz, 1H), 7.81 (m, 1H), 7.55-7.17 (m, 6H), 5.40 (m, 0.3H), 4.99 (m, 0.7H), 3.25 (m, 1H), 2.86 (m, 3.5H), 1.92-0.99 (m, 6H).

### Ex 12: 4-[5-({[(1-Methylpiperid-2-yl)(phenyl)methyl]carbamoyl}amino)-1H-indazol-3-yl]benzenesulfonamide((S,2S),(R,2R))

### Ex 12: 3-Iodo-5-nitro-1H-indazole

In a 250 mL round-bottomed flask equipped with a magnetic bar, 5-nitroindazole (2.7 g, 15.55 mmol) is dissolved in 80 mL of THF at 0° C. Iodine (6.3 g, 24.83 mmol) dissolved in 20 mL of THF and KOH (4.97 g, 88.55 mmol) are successively added. After stirring for 18 hours, the mixture is poured into 100 mL of saturated aqueous Na₂SO₃ solution. Extraction is performed with EtOAc. After drying the organic phase over Na₂SO₄ and evaporating off the solvents, the residue is purified by chromatography on silica gel (eluent: 99/1 to 97/3 DCM/EtOAc) to give 2.33 g of 3-iodo-5-nitro-1H-indazole (M+H)⁺=290.

### Ex 12b: tert-Butyl 3-iodo-5-nitroindazole-1-carboxylate

In a round-bottomed flask equipped with a magnetic bar, 3-iodo-5-nitro-1H-indazole (1.34 g, 4.64 mmol) is dissolved in 50 mL of THF, and triethylamine (0.65 mL, 4.64 mmol), 4-dimethylaminopyridine DMAP (0.116 g, 0.93 mmol) and di-tert-butyl dicarbonate (1.012 g, 4.64 mmol) are successively added. After stirring for 2 hours, the reaction mixture is poured into saturated NH₄Cl solution and extraction is performed with EtOAc. After drying the organic phase over Na₂SO₄ and evaporating off the solvents, the residue is purified by chromatography on silica gel (eluent: 97/3 cyclohexane/EtOAc) to give 0.9 g of tert-butyl 3-iodo-5-nitroindazole-1-carboxylate.

### Ex 12c: tert-Butyl 3-iodo-5-aminoindazole-1-carboxylate

In a round-bottomed flask equipped with a magnetic bar, tert-butyl 3-iodo-5-aminoindazole-1-carboxylate (0.18 g, 0.46 mmol) is dissolved in 20 mL of ethanol. Tin chloride dihydrate (1.5 g, 7.91 mmol) is then added. After stirring for 3 hours, the medium is treated with saturated sodium hydrogen carbonate solution and extracted with EtOAc. After drying the organic phase over Na₂SO₄, the solvents are evaporated off to give 0.095 g of tert-butyl 3-iodo-5-aminoindazole-1-carboxylate. (M+H)⁺=360.

### Ex 12d: tert-Butyl 3-iodo-5-({[(1-methylpiperid-2-yl](phenyl)methyl]carbamoyl}amino)indazole-1-carboxylate((S,2S),(R,2R))

In a round-bottomed flask equipped with a magnetic bar, tert-butyl 3-iodo-5-aminoindazole-1-carboxylate (0.395 g, 0.68 mmol) is dissolved in 30 mL of DCM. Triethylamine (0.4 mL, 2.87 mmol). Triphosgene (0.2 g, 0.67 mmol) dissolved in 5 mL of DCM is added. After stirring for 5 hours, the reaction medium is transferred into a solution of 1-[(1-methylpiperid-2-yl]-1-phenylmethylamine((S,2S),(R,2R)) (0.4 g, 1.16 mmol) and triethylamine (0.4 mL, 2.87 mmol) in 40 mL of DCM. After leaving overnight, the DCM is evaporated off and the residue is purified by chromatography on silica gel (eluent: 94/6 DCM/MeOH) to give 0.357 g of tert-butyl 3-iodo-5-({[(1-methyl piperid-2-yl](phenyl)methyl]carbamoyl}amino)indazole-1-carboxylate((S,2S),(R,2R)). (M+H)⁺=590.

### Ex 12e: 4-[5-({[(1-Methylpiperid-2-yl)(phenyl)methyl]carbamoyl}amino)-1H-indazol-3-yl]benzenesulfonamide((S,2S),(R,2R))

In a round-bottomed flask equipped with a magnetic bar, tert-butyl 3-iodo-5-({[(1-methylpiperid-2-yl](phenyl)methyl]carbamoyl}amino)indazole-1-carboxylate((S,2S),(R,2R)) (0.11 g, 0.19 mmol) is dissolved in 15 mL of DME. 4-Methanesulfonylphenylboronic acid (0.071 g, 0.34 mmol) and tetrakis(triphenylphosphine)palladium (0.017 g, 0.01 mmol) are added. Sodium hydrogen carbonate (0.9 g, 10.71 mmol) is dissolved in 1 mL of water and is added. After refluxing overnight, water is added and the mixture is extracted with DCM. The organic phase is dried over Na₂SO₄ and evaporated. The residue is purified by chromatography on silica gel (eluent: 90/10 DCM/MeOH) to give 0.027 g of 4-[5-({[(1-methylpiperid-2-yl)(phenyl)methyl]carbamoyl}amino)-1H-indazol-3-yl]benzenesulfonamide((S,2S),(R,2R)) and 0.08 g of tert-butyl 3-[4-(aminosulfonyl)phenyl]-5-({[(1-methylpiperid-2-yl)(phenyl)methyl]carbamoyl}amino)-1H-indazole-1-carboxylate((S,2S),(R,2R)). The product obtained is treated with a molar excess of fumaric acid in ethanol. The fumaric acid salt crystallizes after the addition of diisopropyl ether. (M+H)⁺=519. m.p.=220° C. ¹H NMR (DMSO, 200 MHz): δ (ppm) 13.23 (m, 0.5H), 9.02 (s, 1H), 8.25 (d, J=1.4 Hz, 1H), 7.97 (m, 4H), 7.48 (m, 1H), 7.39-7.14 (m, 8H), 6.92 (d, J=7.4 Hz, 1H), 6.57 (s, 2H), 4.87 (t, J=6.8 Hz, 1H), 2.91 (m, 1H), 2.70 (m, 1H), 2.30 (s, 3H), 1.75-1.17 (m, 6H).

### Ex 13: 1-[(1-Methylpiperid-2-yl)(phenyl)methyl]-3-(3-pyrid-4-yl-1H-indazol-5-yl)urea((S,2S),(R,2R))

The compound is prepared by the following the process described in Ex. 12e, replacing the 4-methanesulfonylphenylboronic acid with 4-pyridylboronic acid. The product obtained is treated with a molar excess of fumaric acid in ethanol. The fumaric acid salt crystallizes after the addition of diisopropyl ether. (M+H)⁺=441. m.p.=205° C. ¹H NMR (DMSO, 200 MHz): δ (ppm) 13.37 (m, 0.5H), 9.09 (m, 1H), 8.64 (m, 2H), 8.35 (m, 1H), 7.82 (m, 2H), 7.50 (m, 1H), 7.41-7.15 (m, 7H), 6.94 (m, 1H), 6.58 (s, 1.5H), 5.73 (s, 0.3H (solvent)), 4.89 (t, J=6.8 Hz, 1H), 3.03-2.20 (m, 6H), 1.74-1.14 (m, 6H).

### Ex 14: 1-[(1-Methylpiperid-2-yl)(phenyl)methyl]-3-(3-pyrid-3-yl-1H-indazol-5-yl)urea((S,2S),(R,2R))

The compound is prepared by the following the process described in Ex. 12e, replacing the 4-methanesulfonylphenylboronic acid with 3-pyridylboronic acid. The product obtained is treated with a molar excess of fumaric acid in ethanol. The fumaric acid salt crystallizes after the addition of diisopropyl ether. (M+H)⁺=441. m.p.=210° C. ¹H NMR (DMSO, 200 MHz): δ (ppm) 13.20 (s, 0.6H), 9.06 (d, J=1.8 Hz, 1H), 8.99 (s, 1H), 8.54 (dd, J=4.7, 1.4 Hz, 1H), 8.29-8.15 (m, 2H), 7.57-7.13 (m, 8H), 6.86 (m, 1H), 6.56 (s, 2.3H), 4.86 (m, 1H), 3.01-2.17 (m, 6H), 1.74-1.12 (m, 6H).

### Ex 15: 1-[(1-Methylpiperid-2-yl)(phenyl)methyl]-3-(3-pyrid-2-yl-1H-indazol-5-yl)urea((S,2S),(R,2R))

### Ex 15a: tert-Butyl 5-({[(1-methylpiperid-2-yl)(phenyl)methyl]carbamoyl}amino)-3-pyrid-2-yl-1H-indazole-1-carboxylate((S,2S),(R,2R))

In a round-bottomed flask equipped with a magnetic bar, tert-butyl 3-iodo-5-({[(1-methylpiperid-2-yl](phenyl)methyl]carbamoyl}amino)indazole-1-carboxylate((S,2S),(R,2R)) (0.065 g, 0.11 mmol) is dissolved in 3 mL of THF. Tri-n-butyl(2-pyridyl)stannane (0.081 g, 0.22 mmol) and (dichloro)(diphenylphosphine)palladium (0.020 g, 0.03 mmol) are added. After refluxing for one day, the reaction medium is evaporated to dryness and the residue is purified by chromatography on silica gel (eluent: 98/2 DCM/MeOH) to give 0.032 g of tert-butyl 5-({[(1-methylpiperid-2-yl)(phenyl)methyl]carbamoyl}amino)-3-pyrid-2-yl-1H-indazole-1-carboxylate((S,2S),(R,2R)).

### Ex 15b: 1-[(1-Methylpiperid-2-yl)(phenyl)methyl]-3-(3-pyrid-2-yl-1H-indazol-5-yl)urea((S,2S),(R,2R))

In a round-bottomed flask equipped with a magnetic bar, tert-butyl 5-({[(1-methylpiperid-2-yl)(phenyl)methyl]carbamoyl}amino)-3-pyrid-2-yl-1H-indazole-1-carboxylate((S,2S),(R,2R)) (0.032 g, 0.06 mmol) is dissolved in 3 mL of MeOH and 0.5 M sodium methoxide in MeOH (1.18 mL, 0.59 mmol) is added. After stirring overnight, the reaction medium is evaporated to dryness the residue is purified by chromatography on silica gel (eluent: 95/5 DCM/MeOH) to give 0.012 g of 1-[(1-methylpiperid-2-yl)(phenyl)methyl]-3-(3-pyrid-2-yl-1H-indazol-5-yl)urea((S,2S),(R,2R)). The product obtained is treated with a molar excess of fumaric acid in ethanol. The fumaric acid salt crystallizes after the addition of diisopropyl ether. (M+H)⁺=441. m.p. >250° C. ¹H NMR (DMSO, 200 MHz): δ (ppm) 13.16 (m, 0.6H), 8.92 (s, 1H), 8.64 (m, 1H), 8.52 (s, 1H), 8.09 (dt, J=7.9, 1 Hz, 1H), 7.83 (td, J=7.6, 1.9 Hz, 1H), 7.49-7.17 (m, 9H), 6.80 (d, J=8.8 Hz, 1H), 6.59 (s, 1H), 4.93 (t, J=7.7 Hz, 1H), 3.04 (m. 2H), 1.74-1.16 (m, 6H).

### Ex 16: 1-{(S)-(3,4-Dichlorophenyl)[(2S)-piperid-2-yl]methyl}-3-(3-pyrid-4-yl-1H-indazol-5-yl)urea

### Ex 16a: 3-Iodo-5-nitro-1-{[2-(trimethylsilyl)ethoxy]methyl}-1H-indazole

In a round-bottomed flask equipped with a magnetic bar, 3-iodo-5-nitro-1H-indazole (17.7 g, 61.3 mmol) is dissolved in 300 mL of DCM. Trimethylsilylethoxymethyl chloride (11.9 mL, 67.4 mmol) is added at 0° C. and diisopropylethylamine (12.8 mL, 73.6 mmol) is added dropwise at 0° C. After stirring for 24 hours at RT, the mixture is poured into water and extracted with DCM. After drying the organic phase over Na₂SO₄ and evaporating off the solvents, the residue is purified by chromatography on silica gel (eluent: 95/5 to 80/20 heptane/EtOAc) to give 17.6 g of 3-iodo-5-nitro-1-{[2-(trimethylsilyl)ethoxy]methyl}-1H-indazole.

### Ex 16b: 5-Nitro-3-pyrid-4-yl-1-{[2-(trimethylsilyl)ethoxy]methyl}-1H-indazole

In a round-bottomed flask equipped with a magnetic bar, 3-iodo-5-nitro-1-{[2-(trimethylsilyl)ethoxy]methyl}-1H-indazole (2.5 g, 5.9 mmol) is dissolved in 25 mL of DME. 4-(4,4,5,5-Tetramethyl-1.3.2-dioxaborolan-2-yl)pyridine (1.83 g, 8.94 mmol), a 2M solution of potassium carbonate (7.45 mL. 14.9 mmol) and tetrakis(triphenylphosphine)palladium (0.41 g, 0.36 mmol) are successively added. After refluxing overnight, water is added to the medium and extraction is performed with EtOAc. The organic phase is dried over Na₂SO₄ and evaporated. The residue is purified by chromatography on silica gel (eluent: 90/10 to 70/30 heptane/EtOAc) to give 1.3 g of 5-nitro-3-pyrid-4-yl-1-{[2-(trimethylsilyl)ethoxy]methyl}-1H-indazole.

### Ex 16c: 3-Pyrid-4-yl-1-{[2-(trimethylsilyl)ethoxy]methyl}-1H-indazol-5-amine

In a Parr flask, 5-nitro-3-pyrid-4-yl-1-{[2-(trimethylsilyl)ethoxy]methyl}-1H-indazole (1.3 g, 3.5 mmol) is dissolved in 30 mL of ethanol and palladium-on-charcoal (5%) (0.45 g) is added under N₂. The reaction medium is then stirred under a hydrogen atmosphere for 34 hours. After filtering off the catalyst, the ethanol is evaporated off under vacuum to give 1.1 g of 3-pyrid-4-yl-1-{[2-(trimethylsilyl)ethoxy]methyl}-1H-indazol-5-amine. (M+H)⁺=341.

### Ex 16d: 1-{(S)-(3,4-Dichlorophenyl)[(2S)-piperid-2-yl]methyl}-3-(3-pyrid-4-yl-1H-indazol-5-yl)urea

The compound is prepared by following Example 3 and starting with 3-pyrid-4-yl-1-{[2-(trimethylsilyl)ethoxy]methyl}-1H-indazol-5-amine and (S)-1-[(2S)-1-allylpiperid-2-yl]-1-(3,4-dichlorophenyl)methanamine. (M+H)⁺=497. m.p. (hydrochloride)=195° C. ¹H NMR (DMSO, 200 MHz): δ (ppm) 13.85 (m, 1H), 9.03 (s, 1H), 8.81 (d, J=6.4 Hz, 2H), 8.71 (m, 1H), 8.39 (m, 1H), 8.16 (d, J=6.2 Hz, 2H), 7.77-7.27 (m, 7H), 4.95 (m, 1H), 1.89-1.26 (m, 6H).

### Ex 17: 1-[3-(1H-Benzimidazol-2-yl)-1H-indazol-5-yl]-3-[(1-methylpiperid-2-yl)(phenyl)methyl]urea((S,2S),(R,2R))

### Ex 17a: N-Methoxy-N-methyl-5-nitro-1H-indazole-3-carboxamide

In a round-bottomed flask equipped with a magnetic bar, 5-nitro-1H-indazole-3-carboxylic acid (5.4 g, 26.07 mmol) is dissolved in 100 mL of DMF. Dimethylhydroxylamine (3.18 g, 52.14 mmol), EDC (9.99 g, 52.14 mmol), HOBt (7.04 g, 52.14 mmol) and triethylamine (14.53 mL, 104.28 mmol) are successively added and the reaction mixture is then stirred for 8 days. After evaporating off the DMF, the medium is taken up in water and extracted with EtOAc. The precipitate is filtered off to give 2.9 g of N-methoxy-N-methyl-5-nitro-1H-indazole-3-carboxamide. (M+H)⁺=251.

### Ex 17b: N-Methoxy-N-methyl-5-nitro-1-{[2-(trimethylsilyl)ethoxy]methyl}indazole carboxamide

In a round-bottomed flask equipped with a magnetic bar, N-methoxy-N-methyl-5-nitro-1H-indazole-3-carboxamide (0.33 g, 1.32 mmol) is dissolved in 5 mL of DCM. Trimethylsilylethoxymethyl chloride (0.47 mL, 2.64 mmol) and, dropwise, diisopropylethylamine (0.46 mL, 2.64 mmol) are added at 0° C. After stirring for 2 hours at RT, water is added and the medium is extracted with DCM. After drying the organic phase over Na₂SO₄ and evaporating off the solvents, the residue is purified by chromatography on silica gel (eluent: 95/5 DCM/MeOH) to give 0.51 g of N-methoxy-N-methyl-5-nitro-1-{[2-(trimethylsilyl)ethoxy]methyl}indazole-3-carboxamide. (M+H)⁺=381.

### Ex 17c: 5-Nitro-1-{[2-(trimethylsilyl)ethoxy]methyl}indazolecarbaldehyde

In a round-bottomed flask equipped with a magnetic bar, N-methoxy-N-methyl-5-nitro-1-{[2-(trimethylsilyl)ethoxy]methyl}indazole-3-carboxamide (0.51 g, 1.34 mmol) is dissolved in 20 mL of THF. The mixture is cooled to 0° C. and a 1M solution of diisobutylaluminium hydride (2.3 mL, 2.3 mmol) is added. After stirring for 3 hours, the medium is neutralized with 1.8 mL of acetic acid dissolved in 15 mL of water and extracted with EtOAc. The organic phase is dried over Na₂SO₄ and the solvents are evaporated off to give 0.418 g of 5-nitro-1-{[2-(trimethylsilyl)ethoxy]methyl}indazolecarbaldehyde. (M+H)⁺=322.

### Ex 17d: 3-(1H-Benzimidazol-2-yl)-5-nitro-1-{[2-(trimethylsilyl)ethoxy]methyl}indazole

In a round-bottomed flask equipped with a magnetic bar, 5-nitro-1-{[2-(trimethylsilyl)ethoxy]methyl}-1H-indazolecarbaldehyde (0.41 g, 1.28 mmol) is dissolved in 50 mL of DMF. o-Phenylenediamine (0.138 g, 1.28 mmol) and sulfur (0.049 g, 1.2 mmol) are successively added. The medium is maintained at 100° C. for 4 hours. The DMF is evaporated off and the residue is taken up in water and filtered. The filtrate is taken up in DCM. After evaporation, the residue is purified by chromatography on silica gel (eluent: 7/3 heptane/EtOAc) to give 0.385 g of 3-(1H-benzimidazol-2-yl)-5-nitro-1-{[2-(trimethylsilyl)ethoxy]methyl}indazole.

(M+H)⁺=410.

### Ex 17e: 3-(1H-Benzimidazol-2-yl)-5-amino-1-{[2-(trimethylsilyl)ethoxy]methyl}indazole

In a round-bottomed flask equipped with a magnetic bar, 3-(1H-benzimidazol-2-yl)-5-nitro-1-{[2-(trimethylsilyl)ethoxy]methyl}indazole (0.135 g, 0.33 mmol) and tin chloride dihydrate (0.938 g, 4.94 mmol) are dissolved in 10 mL of EtOAc. After stirring for 3 hours, potassium carbonate is added and the reaction mixture is filtered. The organic phase is taken up in DCM and filtered. The organic phase is dried over Na₂SO₄ and evaporated to give 0.12 g of 3-(1H-benzimidazol-2-yl)-5-amino-1-{[2-(trimethylsilyl)ethoxy]methyl}indazole. (M+H)⁺=380.

### Ex 17f: 1-[3-(1H-Benzimidazol-2-yl)-1-{[2-(trimethylsilyl)ethoxy]methyl}indazol-5-yl]-3-[(1-methylpiperid-2-yl)(phenyl)methyl]urea((S,2S),(R,2R))

This compound is prepared according to the process described in Example 12d starting with 0.12 g of 3-(1H-benzimidazol-2-yl)-5-amino-1-{[2-(trimethylsilyl)ethoxy]methyl}indazole. 0.066 g of 1-[3-(1H-benzimidazol-2-yl)-1-{[2-(trimethylsilyl)ethoxy]methyl}indazol-5-yl]-3-[(1-methylpiperid-2-yl)(phenyl)methyl]urea((S,2S),(R,2R)) is obtained. (M+H)⁺=610.

### Ex 17g: 1-[3-(1H-Benzimidazol-2-yl)-1H-indazol-5-yl]-3-[(1-methylpiperid-2-yl)(phenyl)methyl]urea((S,2S),(R,2R))

In a round-bottomed flask equipped with a magnetic bar, 1-[3-(1H-benzimidazol-2-yl)-1-{[2-(trimethylsilyl)ethoxy]methyl-1H-indazol-5-yl]-3-[(S)-[(2S)-1-methylpiperid-2-yl](phenyl)methyl]urea (0.064 g, 0.1 mmol) is dissolved in 2 mL of DCM. 4N HCl in dioxane (0.6 mL, 2.41 mmol) is added. After stirring overnight, the solvents are evaporated off and the residue is partitioned between a solution of NaOH and a mixture of DCM/isopropanol. The organic phase is evaporated and the residue is purified by chromatography on silica gel (eluent: 90/10 DCM/MeOH) to give 0.012 g of 1-[3-(1H-benzimidazol-2-yl)-1H-indazol-5-yl]-3-[(1-methylpiperid-2-yl)(phenyl)methyl]urea((S,2S),(R,2R)). The product obtained is treated with a molar excess of fumaric acid in ethanol. The fumaric acid salt crystallizes after the addition of diisopropyl ether. (M+H)⁺=480. m.p.=250° C. ¹H NMR (DMSO, 200 MHz): 13.40 (s, 1H), 12.81 (s, 1H), 9.08 (s, 1H), 8.47 (s, 1H), 7.76-7.05 (m, 12H), 6.82 (d, J=7.8 Hz, 1H), 6.57 (s, 2H), 4.89 (m, 1H), 3.03-2.20 (m, 6H), 1.76-1.11 (m, 6H).

### Ex 18: 3-Methyl-5-({[(1-methylpiperid-2-yl)(phenyl)methyl]carbamoyl}amino)-1H-indazole ((S,2S),(R,2R))

### Ex 18a: 3-Methyl-5-nitro-1H-indazole

In a round-bottomed flask equipped with a magnetic bar, 1-(2-fluoro-5-nitrophenyl)ethanone (9.72 g, 53.1 mmol) is dissolved in 25 mL of ethylene glycol. After addition of hydrazine (2.71 mL, 87.1 mmol), the reaction medium is stirred for 30 minutes it is then heated for 4 hours at 165° C. The resulting mixture is allowed to cool to RT. The medium is filtered to give a solid. The filtrate is taken up in DCM and washed twice with water. The organic phase is dried over Na₂SO₄ and evaporated to give a solid. The two solids are then combined to give 8.65 g of 3-methyl-5-nitro-1H-indazole. (M+H)⁺=178.

### Ex 18b: tert-Butyl 3-methyl-5-nitroindazole-1-carboxylate

In a round-bottomed flask equipped with a magnetic bar, 3-methyl-5-nitro-1H-indazole (3 g, 16.93 mmol), di-tert-butyl dicarbonate (3.69 g, 16.93 mmol), triethylamine (2.36 mL, 16.93 mmol) and DMAP (0.414 g, 3.39 mmol) are dissolved in 100 mL of THF. After stirring for 3 hours, the reaction medium is taken up in EtOAc and washed with saturated NH₄Cl solution and then with brine. The organic phase is dried over Na₂SO₄ and evaporated to give 4.55 g of tert-butyl 3-methyl-5-nitroindazole-1-carboxylate. (M+H)⁺=278.

### Ex 18c: tert-Butyl 5-amino-3-methylindazole-1-carboxylate

In a Parr flask, tert-butyl 3-methyl-5-nitroindazole-1-carboxylate (0.6 g, 2.16 mmol) is dissolved in 120 mL of MeOH and palladium-on-charcoal (5%) (0.12 g, 1.12 mmol) is then added under N₂. The reaction medium is stirred under 3 atm of hydrogen for 34 hours. After filtration and evaporating off the MeOH, the residue is purified by chromatography on silica gel (eluent: 95/5 DCM/MeOH) to give 0.45 g of tert-butyl 5-amino-3-methylindazole-1-carboxylate. (M+H)⁺=248.

### Ex18d: tert-Butyl 3-methyl-5-({[(1-methylpiperid-2-yl)(phenyl)methyl]carbamoyl}amino)indazole-1-carboxylate((S,2S),(R,2R))

In a round-bottomed flask equipped with a magnetic bar, tert-butyl 5-amino-3-methylindazole-1-carboxylate (0.25 g, 1.01 mmol) is dissolved in 10 mL of DCM at 0° C. Triethylamine (0.18 mL, 1.31 mmol) and triphosgene (0.2 g, 0.67 mmol) are added to the reaction medium. After stirring for 3 hours at RT, 1-(1-methylpiperid-2-yl)-1-phenylmethylamine((S,2S),(R,2R)) (0.310 g, 1.52 mmol) is added as a bolus. After stirring overnight, the reaction medium is partitioned between 100 mL of DCM and 20 mL of saturated NaHCO₃ solution. The organic phase is successively washed with water and brine. After drying the aqueous phase over Na₂SO₄ and evaporation, the residue is purified by chromatography on silica gel (eluent: 93/7 DCM/MeOH) to give 0.23 g of tert-butyl 3-methyl-5-({[1-methylpiperid-2-yl](phenyl)methyl]carbamoyl}amino)indazole-1-carboxylate((S,2S),(R,2R)). (M+H)⁺=478.

### Ex 18e: 3-Methyl-5-({[(1-methylpiperid-2-yl)(phenyl)methyl]carbamoyl}amino)-1H-indazole((S,2S),(R,2R))

In a round-bottomed flask equipped with a magnetic bar, 4 mL of a 4N solution of HCl in dioxane are added to tert-butyl 3-methyl-5-({[(1-methyl piperid-2-yl)(phenyl)methyl]carbamoyl}amino)indazole-1-carboxylate((S,2S),(R,2R)) (0.225 g, 0.47 mmol). After stirring overnight, 0.2 mL of aqueous 30% ammonium hydroxide solution is added. The reaction medium is evaporated with 0.5 g of silica to give a solid deposit, which is used in a chromatography on silica gel (eluent: DCM/MeOH, 100/0 to 90/10) to give 0.117 g of 3-methyl-5-({[(1-methylpiperid-2-yl)(phenyl)methyl]carbamoyl}amino)-1H-indazole((S,2S),(R,2R)). The product obtained is treated with a molar excess of fumaric acid in ethanol. The fumaric acid salt crystallizes after the addition of diisopropyl ether. (M+H)⁺=378. m.p.=200-201° C., ¹H NMR (DMSO, 200 MHz): δ (ppm) 8.89 (s, 1H), 7.79 (m, 1H), 7.39-7.00 (m, 9H), 6.57 (s, 2H), 4.86 (t, J=7.2 Hz, 1H), 2.96 (m, 1H), 2.80 (m, 1H), 2.54 (m, 1H), 2.36 (s, 3H), 1.72-1.15 (m, 6H).

### Ex 19: N-[7-Fluoro-5-({[(1-methylpiperid-2-yl)(phenyl)methyl]carbamoyl}amino)-1H-indazol-3-yl]benzamide((S,2S),(R,2R))

### Ex 19a: 7-Fluoro-1H-indazol-3-ylamine

In a three-necked flask equipped with a magnetic stirrer and placed under argon, 2,3-difluorobenzonitrile (1 g, 7.19 mmol) is dissolved in ethanol (25 mL) and hydrazine hydrate (0.35 mL, 7.19 mmol) is added. The reaction medium is refluxed for 3 hours. Addition of further hydrazine hydrate (0.35 mL, 7.19 mmol) and refluxing for a further 16 hours allow the reaction to be completed. After stopping the heating and cooling to RT, the reaction medium is concentrated to dryness under RP and the residue is taken up in water (35 mL) and EtOAc (50 mL). After separation, the aqueous phase is extracted with EtOAc (35 mL). The combined organic extracts are washed with water (35 mL) and then brine (35 mL), dried over MgSO₄, filtered and concentrated under RP. The solid thus isolated is concreted with isopropyl ether, filtered, washed with isopropyl ether and dried. 0.89 g of 7-fluoro-1H-indazol-3-ylamine is isolated in the form of a glossy beige-coloured solid. m.p.=170° C.

### Ex 19b: N-(7-Fluoro-1H-indazol-3-yl)benzamide

In a three-necked flask equipped with a magnetic stirrer and placed under argon, 7-fluoro-1H-indazol-3-ylamine (0.5 g, 3.33 mmol) is dissolved in pyridine (5 mL). The solution is cooled to about 0° C. using an ice bath. Benzoyl chloride (0.384 mL, 3.33 mmol) is added dropwise while maintaining the temperature between 0° C. and 5° C., and stirring is continued at about 0° C. for 15 minutes after the end of addition. After cooling and stirring for 1 hour at RT, the reaction medium is hydrolysed with 20 mL of water and extracted with twice 20 mL of EtOAc. The combined organic extracts are dried over MgSO₄, filtered and concentrated to dryness the yellow solid isolated is taken up in methylene chloride and the insoluble matter is isolated by filtration, washed with isopropyl ether and dried to give 0.62 g of N-(7-fluoro-1H-indazol-3-yl)benzamide in the form of a pale yellow solid. m.p.=232° C.

### Ex 19c: N-(7-Fluoro-5-nitro-1H-indazol-3-yl)benzamide

In a three-necked flask equipped with a magnetic stirrer and placed under argon, N-(7-fluoro-1H-indazol-3-yl)benzamide (0.15 g, 0.59 mmol) is suspended in acetonitrile (10 mL). The solution is cooled to about 0° C. using an ice bath. Nitronium tetrafluoroborate (0.186 g, 1.17 mmol) is added in a single portion and the reaction medium is stirred for 1 hour at about 0° C. The medium is then hydrolysed with saturated aqueous sodium hydrogen carbonate solution (10 mL) and extracted with twice 20 mL of EtOAc. The combined organic extracts are washed with water (20 mL), dried over MgSO₄, filtered and concentrated to dryness. The isolated solid is taken up in isopropyl ether (5 mL), filtered, washed and dried to give 0.12 g of N-(7-fluoro-5-nitro-1H-indazol-3-yl)benzamide in the form of a beige-coloured solid that melts. m.p. (fumarate)>260° C.

### Ex 19d: N-(5-Amino-7-fluoro-1H-indazol-3-yl)benzamide

An autoclave is successively charged with N-(7-fluoro-5-nitro-1H-indazol-3-yl)benzamide (3.08 g; 10.2 mmol), Raney nickel (500 mg) and ethanol (100 mL). The apparatus is then purged with nitrogen (three times), with hydrogen (twice) and placed under pressure of 5 bar of H₂ at a temperature of 45° C. for 16 hours. After cooling to room temperature and pressure, the reaction medium is filtered through a bed of Celite 545. After rinsing the Celite with 500 mL of ethanol, the filtrate is concentrated to dryness. The isolated solid is purified by chromatography on silica 40-65 μm contained in a column 6 cm in diameter, eluting with pure EtOAc. Collection and evaporation of the fractions containing the product give 1.15 g of N-(5-amino-7-fluoro-1H-indazol-3-yl)benzamide in the form of a brown solid of Rf 0.56 (silica plate, eluent: pure EtOAc). m.p.=208° C.

### Ex19e: N-[7-Fluoro-5-({[(1-methylpiperid-2-yl)(phenyl)methyl]carbamoyl}amino)-1H-indazol-3-yl]benzamide((S,2S),(R,2R))

In a round-bottomed flask equipped with a magnetic bar, N-(5-amino-7-fluoro-1H-indazol-3-yl)benzamide (2.5 g, 9.25 mmol), triethylamine (1.29 mL, 9.25 mmol) and para-nitrophenyl chloroformate (1.864 g, 9.25 mmol) are dissolved in 28 mL of THF. After stirring overnight, the solution is transferred into an 80 mL microwave tube. 1-[(1-Methylpiperid-2-yl]-1-phenylmethylamine((S,2S),(R,2R)) (1.89 g, 9.25 mmol) is then added and the tube is placed in a microwave machine, where it is subjected to an initial power of 150 watts to maintain it at 100° C. for 35 min. A pressure of 2 bar develops in the reactor. The reaction medium is evaporated and purified by chromatography on silica gel (eluent: 95/5/0.5 to 90/10/1 DCM/MeOH/NH₄OH) to give 2.41 g N-[7-fluoro-5-({[(1-methylpiperid-2-yl)(phenyl)methyl]carbamoyl}amino)-1H-indazol-3-yl]benzamide((S,2S),(R,2R)). The product obtained is treated with a molar excess of fumaric acid in ethanol. The fumaric acid salt crystallizes after the addition of diisopropyl ether. (M+H)⁺=501. m.p.=164° C. ¹H NMR (DMSO, 200 MHz): δ (ppm) 13.14 (m, 0.6H), 10.69 (s, 1H), 9.14 (s, 1H), 8.02 (m, 2H), 7.65-7.12 (m, 10H), 6.98 (d, J=7.3 Hz, 1H), 6.56 (s, 2H), 4.81 (t, J=7 Hz, 1H), 2.90 (m, 1H), 2.71 (m, 1H), 2.29 (s, 3H), 1.72-1.10 (m, 6H).

### Ex 20: 1-(3-Amino-7-fluoro-1H-indazol-5-yl)-3-[(1-methylpiperid-2-yl)(phenyl)methyl]urea((S,2S),(R,2R))

In a round-bottomed flask equipped with a magnetic stirrer and a condenser, a solution of N-[7-fluoro-5-({[(1-methylpiperid-2-yl)(phenyl)methyl]carbamoyl}amino)-1H-indazol-3-yl]benzamide((S,2S),(R,2R)) (1 g, 2 mmol) in 6.7 mL of 6N HCl is refluxed. The medium is evaporated, taken up in DCM and MeOH, neutralized to pH=7 with aqueous 30% ammonium hydroxide solution and re-evaporated in the presence of 3 g of silica. The solid deposit is chromatographed on silica gel (eluent: 99/9/1 to 93/7/0.3 DCM/MeOH/NH₄OH) to give 0.37 g of 1-(3-amino-7-fluoro-1H-indazol-5-yl)-3-[(1-methylpiperid-2-yl)(phenyl)methyl]urea((S,2S),(R,2R)). The product obtained is treated with a molar excess of fumaric acid in ethanol. The fumaric acid salt crystallizes after the addition of diisopropyl ether. (M+H)⁺=397. m.p.=221-222° C. ¹H NMR (DMSO, 200 MHz): δ (ppm) 8.90 (s, 1H), 7.43 (d, J=1.5 Hz, 1H), 7.39-7.17 (m, 7H), 6.99 (d, J=7 Hz, 1H), 6.60 (s, 2.2H), 4.87 (t, J=7 Hz, 1H), 2.94 (m, 1H), 2.73 (m, 1H), 2.34 (s, 3H), 1.75-1.21 (m, 6H).

### Ex 21: 1-(7-Fluoro-1H-indazol-5-yl)-3-[(1-methylpiperid-2-yl)(phenyl)methyl]urea((S,2S),(R,2R))

In a round-bottomed flask equipped with a magnetic stirrer, 1-(3-amino-7-fluoro-1H-indazol-5-yl)-3-[(1-methylpiperid-2-yl)(phenyl)methyl]urea((S,2S),(R,2R)) (0.25 g, 0.63 mmol) is dissolved in 5 mL of 30% HCl solution at 0° C. Sodium nitrite (0.043 g, 0.63 mmol) is added in a minimum amount of water. After stirring for 15 minutes, hypophosphorous acid (1.04 mL, 9.46 mmol) is added and stirring is continued overnight. The reaction medium is neutralized with 10% NaOH solution and extracted with a 9/1 DCM/MeOH mixture. After evaporating the organic phase, the residue is chromatographed on silica gel (eluent: 99/9/1 to 92/8/0.2 DCM/MeOH/NH₄OH) to give 0.02 g of 1-(7-fluoro-1H-indazol-5-yl)-3-[(−1-methylpiperid-2-yl)(phenyl)methyl]urea((S,2S),(R,2R)). The product obtained is treated with a molar excess of fumaric acid in ethanol. The fumaric acid salt crystallizes after the addition of diisopropyl ether. (M+H)⁺=382. m.p.=224-227° C. ¹H NMR (DMSO, 200 MHz): δ (ppm) 13.29 (m, 0.7H), 9.04 (s, 1H), 7.99 (d, J=3.4 Hz, 1H), 7.51 (d, J=1.5 Hz, 1H), 7.37-7.13 (m, 7H), 6.94 (d, J=7.6 Hz, 1H), 6.56 (s, 1.2H), 4.83 (t, J=6.5 Hz, 1H), 2.88 (m, 1H), 2.64 (m, 1H), 2.26 (s, 3H), 1.73-1.13 (m, 6H).

### Ex 22: 1-{3-[(3-Fluorophenyl)ethynyl]-1H-indazol-5-yl}-3-[(1-methylpiperid-2-yl)(phenyl)methyl]urea((S,2S),(R,2R))

### Ex 22a: 3-[(3-Fluorophenyl)ethynyl]-5-nitro-1-{[2-(trimethylsilyl)ethoxy]methyl}-1H-indazole

In a round-bottomed flask equipped with a magnetic bar, 3-iodo-5-nitro-1-{[2-(trimethylsilyl)ethoxy]methyl}-1H-indazole (0.263 g, 0.63 mmol) is dissolved in 3 mL of DMF. CuI (0.018 g, 0.09 mmol), PdCl₂(PPh₃)₂ (0.0264 g, 0.04 mmol), triethylamine (0.17 mL, 1.25 mmol) and 3-fluorophenylacetylene (0.12 mL, 0.123 mmol) are successively added. The reaction medium is heated at 90° C. for 15 hours. The DMF is evaporated off and the residue is partitioned between water and EtOAc. The organic phase is dried over Na₂SO₄ and evaporated. The residue is purified by chromatography on silica gel (eluent: 8/2 heptane/EtOAc) to give 0.068 g of 3-[(3-fluorophenyl)ethynyl]-5-nitro-1-{[2-(trimethylsilyl)ethoxy]methyl}-1H-indazole. (M+H)⁺=412.

### Ex 22b: 5-Amino-3-[(3-fluorophenyl)ethynyl]-1-{[2-(trimethylsilyl)ethoxy]methyl}-1H-indazole

Prepared starting with 3-[(3-fluorophenyl)ethynyl]-5-nitro-1-{[2-(trimethylsilyl)ethoxy]methyl}-1H-indazole according to the process described in Ex. 17e. (M+H)⁺=382

### Ex 22c: 1-{3-[(3-Fluorophenyl)ethynyl]-1-{[2-(trimethylsilyl)ethoxy]methyl}1H-indazol-5-yl}-3-[(1-methylpiperid-2-yl)(phenyl)methyl]urea((S,2S),(R,2R))

Prepared starting with 1-[(1-methylpiperid-2-yl]-1-phenylmethylamine((S,2S),(R,2R)) and 5-amino-3-[(3-fluorophenyl)ethynyl]-1-{[2-(trimethylsilyl)ethoxy]methyl}-1H-indazole prepared in Ex. 22b according to the process described in Ex. 1a. (M+H)⁺=612.

### Ex 22d: 1-{3-[(3-Fluorophenyl)ethynyl]-1H-indazol-5-yl}-3-[(1-methylpiperid-2-yl)(phenyl)methyl]urea((S,2S),(R,2R))

Prepared according to the process described in Ex. 1b. The product obtained is treated with a molar excess of fumaric acid in ethanol. The fumaric acid salt crystallizes after the addition of diisopropyl ether. (M+H)⁺=481. m.p.=260° C. ¹H NMR (DMSO, 200 MHz): δ (ppm) 13.38 (s, 1H), 9.09 (s, 1H), 7.98 (d, J=1.3 Hz, 1H), 7.56-7.17 (m, 12H), 7.02 (d, J=8.2 Hz, 1H), 6.59 (s, 0.7H), 4.96 (m, 1H), 2.62 (m, 1H), 1.74-1.11 (m, 6H).

The compounds of Examples 23 to 25 were prepared according to the process described in Ex. 22.

### Ex 23: 1-[(1-Methylpiperid-2-yl)(phenyl)methyl]-3-[3-(phenylethynyl)-1H-indazol-5-yl]urea((S,2S),(R,2R))

Prepared replacing in Ex. 22b the 3-fluorophenylacetylene with phenylacetylene. The product obtained is treated with a molar excess of fumaric acid in ethanol. The fumaric acid salt crystallizes after the addition of diisopropyl ether. (M+H)⁺=464. m.p.=227° C. ¹H NMR (DMSO, 200 MHz): δ (ppm) 13.28 (s, 0.7H), 9.05 (s, 1H), 7.99 (d, J=1.4 Hz, 1H), 7.62-7.13 (m, 13H), 6.89 (d, J=8 Hz, 1H), 6.57 (s, 1.5H), 4.84 (t, J=6.5 Hz, 1H), 3.00-2.18 (m, 6H), 1.77-1.10 (m, 6H).

### Ex 24: 1-{3-[(4-Fluorophenyl)ethynyl]-1H-indazol-5-yl}-3-[(1-methylpiperid-2-yl)(phenyl)methyl]urea((S,2S),(R,2R))

Prepared replacing in Ex. 22b the 3-fluorophenylacetylene with 4-fluorophenylacetylene. The product obtained is treated with a molar excess of fumaric acid in ethanol. The fumaric acid salt crystallizes after the addition of diisopropyl ether. (M+H)⁺=482. m.p.=244° C. ¹H NMR (DMSO, 200 MHz): δ (ppm) 13.28 (s, 0.6H), 9.05 (s, 1H), 7.99 (d, J=1.4 Hz, 1H), 7.63 (m, 1H), 4.45 (m, 1H), 7.38-7.14 (m, 8H), 6.91 (d, J=8.2 Hz, 1H), 6.57 (s, 1H), 4.87 (m, 1H), 3.00-2.19 (m, 6H), 1.74-1.11 (m, 6H).

### Ex 25: 1-[(1-Methylpiperid-2-yl)(phenyl)methyl]-3-[3-(pyrid-3-ylethynyl)-1H-indazol-5-yl]urea((S,2S),(R,2R))

Prepared replacing in Ex. 22b the 3-fluorophenylacetylene with 3-pyridylacetylene. The product obtained is treated with a molar excess of fumaric acid in ethanol. The fumaric acid salt crystallizes after the addition of diisopropyl ether. (M+H)⁺=465. m.p.=230° C. ¹H NMR (DMSO, 200 MHz): δ (ppm) 13.37 (s, 0.6H), 9.06 (s, 1H), 8.77 (s, 1H), 8.58 (d, J=5 Hz, 1H), 8.04-7.96 (m, 2H), 7.45 (m, 2H), 7.38-7.14 (m, 6H), 6.92 (d, J=7.5 Hz, 1H), 6.57 (s, 2.1H), 4.85 (t, J=6.6 Hz, 1H), 3.02-2.18 (m, 6H), 1.75-1.12 (m, 6H).

### Ex 26: 1-{(S)-(3,4-Dichlorophenyl)[(2S)-piperid-2-yl]methyl}-3-[3-(phenylethynyl)-1H-indazol-5-yl]urea

The compound was prepared starting with (S)-1-[(2S)-1-allylpiperid-2-yl]-1-(3,4-dichlorophenyl)methanamine and 5-amino-3-(phenylethynyl)-1-{[2-(trimethylsilyl)ethoxy]methyl}indazole prepared in Ex. 23 according to the process described in Example 3. The product obtained is treated with a molar excess of fumaric acid in ethanol. The fumaric acid salt crystallizes after the addition of diisopropyl ether. (M+H)⁺=518. m.p.=160° C. ¹H NMR (DMSO, 200 MHz): δ (ppm) 13.29 (s, 0.7H), 9.53 (s, 1H), 8.03-7.88 (m, 2H), 7.67-7.22 (m, 10H), 6.56 (s, 3.1H), 4.81 (t, J=7.8 Hz, 1H), 3.25-2.55 (m, 3H), 1.80-1.12 (m, 6H).

### Ex 27: 1-[(1-Methylpiperid-2-yl)(phenyl)methyl]-3-[3-(2-phenylethyl)-1H-indazol-5-yl]urea((S,2S),(R,2R))

### Ex 27a: 5-Amino-1-{[2-(trimethylsilyl)ethoxy]methyl}-3-phenylindazole

In a Parr flask, 5-nitro-1-{[2-(trimethylsilyl)ethoxy]methyl}-3-phenethynylindazole (0.135 g, 0.34 mole) is dissolved in 20 mL of ethanol and palladium-on-charcoal (0.375 g, 3.468 mmol) is added under N₂. The reaction medium is stirred under 1 atm of hydrogen for 24 hours. After filtering off the catalyst and evaporating off the ethanol, 0.12 g of 5-amino-1-{[2-(trimethylsilyl)ethoxy]methyl}-3-phenethylylindazole is recovered. (M+H)⁺=368.

### Ex 27 b: 1-[(1-Methylpiperid-2-yl)(phenyl)methyl]-3-[3-(2-phenylethyl)-1H-indazol-5-yl]urea((S,2S),(R,2R))

The compound was prepared starting with 1-[(1-methylpiperid-2-yl]-1-phenylmethylamine((S,2S),(R,2R)) and 5-amino-1-{[2-(trimethylsilyl)ethoxy]methyl}-3-phenethylindazole prepared in Ex. 27a according to the process described in Ex. 1. The product obtained is treated with a molar excess of fumaric acid in ethanol. The fumaric acid salt crystallizes after the addition of diisopropyl ether. (M+H)⁺=468. m.p.=205° C. ¹H NMR (DMSO, 200 MHz): δ (ppm) 12.40 (m, 1.3H), 8.78 (s, 1H), 7.83 (s, 1H), 7.37-7.09 (m, 12H), 6.76 (d, J=7.4 Hz, 1H), 6.56 (s, 1.4H), 4.85 (t, J=6.4 Hz, 1H), 3.17-2.76 (m, 7H), 2.25 (s, 3H), 1.75-1.15 (m, 6H).

### Ex 28: N-[5-({[(1-Methylpiperid-2-yl)(phenyl)methyl]carbamoyl}amino)-1H-indazol-3-yl]benzamide((S,2S),(R,2R))

### Ex 28a: tert-Butyl 3-(Benzoylamino)-5-nitroindazole-1-carboxylate

In a round-bottomed flask equipped with a magnetic bar, tert-butyl 3-amino-5-nitroindazole-1-carboxylate (1 g, 3.59 mmol) is dissolved in 11 mL of pyridine at 0° C. Benzoyl chloride (0.46 mL, 3.95 mmol) is added and the mixture is stirred overnight. The reaction medium is partitioned between solvent and water. The organic phase is washed with aqueous 0.5 N HCl solution. The organic phase is dried over Na₂SO₄ and evaporated. The residue is purified by chromatography on silica gel (eluent: 7/3 heptane/ethyl acetate EtOAc) to give 0.404 g of tert-butyl 3-(benzoylamino)-5-nitroindazole-1-carboxylate. (M+H)⁺=383.

### Ex 28b: tert-Butyl 5-amino-3-(benzoylamino)indazole-1-carboxylate

This compound is prepared according to the process described in Example 27a starting with tert-butyl 3-(benzoylamino)-5-nitroindazole-1-carboxylate (0.4 g, 1.05 mmol). 0.32 g of tert-butyl 5-amino-3-(benzoylamino)indazole-1-carboxylate are thus obtained. (M+H)⁺=353.

### Ex 28c: tert-Butyl 3-(benzoylamino)-5-({[(1-methylpiperid-2-yl)(phenyl)methyl]carbamoyl}amino)-indazole-1-carboxylate((S,2S),(R,2R))

The compound was prepared starting with 1-[(1-methylpiperid-2-yl]-1-phenylmethylamine ((S,2S),(R,2R)) (0.136 g, 0.67 mmol) and tert-butyl 5-amino-3-(benzoylamino)indazole-1-carboxylate (0.160 g, 0.39 mmol) according to the process of Ex. 18d. 0.025 g of tert-butyl 3-(benzoylamino)-5-({[(1-methylpiperid-2-yl)(phenyl)methyl]carbamoyl}amino)-indazole-1-carboxylate is obtained ((S,2S),(R,2R)). (M+H)⁺=583.

### Ex 28d: N-[5-({[(1-Methylpiperid-2-yl)(phenyl)methyl]carbamoyl}amino)-1H-indazol-3-yl]benzamide((S,2S),(R,2R))

In a round-bottomed flask equipped with a magnetic stirrer, tert-butyl 3-(benzoylamino)-5-({[(1-methylpiperid-2-yl)(phenyl)methyl]carbamoyl}amino)-indazole-1-carboxylate((S,2S),(R,2R)) (0.031 g, 0.05 mmol) is dissolved in 8 mL of dioxane. A 4M solution of HCl in dioxane is added (0.53 mL, 0.5 mmol) and the reaction medium is stirred for 2 hours. The dioxane is evaporated off and the residue is partitioned between a sodium hydroxide solution and a mixture of DCM/isopropanol. The organic phase is evaporated and the residue is purified by chromatography on silica gel (eluent: 95/5 DCM/MeOH) to give 0.023 g of N-[5-({[(1-methylpiperid-2-yl)(phenyl)methyl]carbamoyl}amino)-1H-indazol-3-yl]benzamide((S,2S),(R,2R)). (M+H)⁺=483, m.p. (fumarate)=235° C. ¹H NMR (DMSO, 200 MHz): δ (ppm) 12.55 (s, 1H), 10.57 (s, 1H), 8.84 (s, 1H), 8.03 (m, 2H), 7.71 (s, 1H), 7.63-7.44 (m, 3H), 7.39-7.11 (m, 7H), 6.72 (d, J=7.9 Hz, 1H), 6.56 (s, 1.5H), 4.81 (t, J=6.5 Hz, 1H), 2.88 (m, 1H), 2.23 (s, 3H), 1.73-1.14 (m, 6H).

### Ex 29: 1-(3-Amino-1H-indazol-5-yl)-3-{(S)-(3,4-dichlorophenyl)[(2S)-piperid-2-yl]methyl}urea

### Ex 29a: tert-Butyl 3-[bis(tert-butoxycarbonyl)amino]-5-nitro-1H-indazole-1-carboxylate

In a round-bottomed flask equipped with a magnetic bar, 5-nitro-1H-indazol-3-amine (4.00 g, 22.6 mmol) is dissolved in 50 mL of DCM. Triethylamine (11.0 mL, 79.2 mmol), DMAP (1.4 g, 11.3 mmol) and di-tert-butyl dicarbonate (17.3 g, 79.2 mmol) are successively added to the mixture. After stirring for 18 hours, further DCM is added and the resulting mixture is washed with saturated NH₄Cl solution and then with saturated NaCl solution. After drying the organic phase over Na₂SO₄ and evaporating off the solvents, the residue is purified by chromatography on silica gel (eluent: 9/1 and then 4/1 heptane/EtOAc) to give 9.5 g of tert-butyl 3-[bis(tert-butoxycarbonyl)amino]-5-nitro-1H-indazole-1-carboxylate. (M+H)⁺=479

### Ex 29b: tert-Butyl 5-amino-3-[bis(tert-butoxycarbonyl)amino]-1H-indazole-1-carboxylate

In a Parr flask, tert-butyl 3-[bis(tert-butoxycarbonyl)amino]-5-nitro-1H-indazole-1-carboxylate (9.5 g, 19.9 mmol) is dissolved in 200 mL of ethanol and 5% palladium-on-charcoal (0.845 g) is added under N₂. The reaction medium is stirred under a hydrogen atmosphere for 4 hours. After filtering off the catalyst and evaporating off the ethanol, 8.6 g of tert-butyl 5-amino-3-[bis(tert-butoxycarbonyl)amino]-5-nitro-1H-indazole-1-carboxylate are recovered. (M+H)⁺=449

### Ex 29c: 5-tert-Butyl ({[(S)-[(2S)-1-allylpiperid-2-yl](3,4-dichlorophenyl)methyl]carbamoyl}amino)-3-[bis(tert-butoxycarbonyl)amino]-1H-indazole-1-carboxylate

This compound is prepared starting with (0.35 g, 0.79 mmol) and (S)-1-[(2S)-1-allylpiperid-2-yl]-1-(3,4-dichlorophenyl)methanamine (0.35 g, 1.2 mmol) according to the process described in Example 3a. (M+H)⁺=773

### Ex 29d: tert-Butyl 3-[bis(tert-butoxycarbonyl)amino]-5-[({(S)-(3,4-dichlorophenyl)[(2S)-piperid-2-yl]methyl}carbamoyl)amino]-1H-indazole-1-carboxylate

Prepared starting with tert-butyl 5-({[(S)-[(2S)-1-allylpiperid-2-yl](3,4-dichlorophenyl)methyl]-carbamoyl}amino)-3-[bis(tert-butoxycarbonyl)amino]-1H-indazole-1-carboxylate (0.5 g, 0.64 mmol) according to the process of Ex. 3b. (M+H)⁺=733

### Ex 29e: 1-(3-Amino-1H-indazol-5-yl)-3-{(S)-(3,4-dichlorophenyl)[(2S)-piperid-2-yl]methyl}urea

In a round-bottomed flask equipped with a magnetic stirrer, tert-butyl 3-[bis(tert-butoxycarbonyl)amino]-5-[({(S)-(3,4-dichlorophenyl)[(2S)-piperid-2-yl]methyl}carbamoyl)amino]-1H-indazole-1-carboxylate (0.19 g, 0.25 mmol) is dissolved in dioxane (2.5 mL). A 4N solution of HCl in dioxane (2.5 mL, 10 mmol) is then added. The reaction mixture is stirred for 3 hours at RT and concentrated under vacuum. The residue is purified by chromatography on silica gel diluted with a DCM/MeOH/NH₄OH mixture (98/2/0.2 to 90/10/1). 0.07 g of oil is obtained, which is treated with a 0.2N solution of HCl in ethyl ether. After filtration, 1-(3-amino-1H-indazol-5-yl)-3-{(S)-(3,4-dichlorophenyl)[(2S)-piperid-2-yl]methyl}urea is obtained in the form of the dihydrochloride. (M+H)⁺=432, m.p.=214° C. ¹H NMR (DMSO, 200 MHz): δ (ppm) 8.95 (s, 1H), 8.80-8.50 (m, 2H), 7.90 (s, 1H), 7.70 (m, 2H), 7.50-7.30 (m, 4H), 4.90 (t, 1H), 1.85-1.20 (m, 6H).

### Ex 30: 1-(1H-Indazol-5-yl)-3-[(1-methylpiperid-2-yl)phenylmethyl]-1,3-dihydroimidazol-2-one((S,2S),(R,2R))

### Ex 30a: 2,2-(Dimethoxyethyl)-[(1-methylpiperid-2-yl)phenylmethyl]amine((S,2S),(R,2R))

In a round-bottomed flask equipped with a magnetic bar, 1-[1-methylpiperid-2-yl]-1-phenylmethylamine((S,2S),(R,2R)) (0.4 g, 1.96 mmol) is dissolved in 6 mL of THF. 2,2-Dimethoxyethanal (0.37 mL, 2.45 mmol) and sodium triacetoxyborohydride (0.622 g, 2.94 mmol) are successively added. After stirring overnight, the reaction medium is partitioned between saturated sodium bicarbonate solution and DCM. The organic phase is recovered and chromatographed on silica gel (eluent: 98/2/0.2 DCM/MeOH/NH₄OH) to give 0.363 g of 2,2-(dimethoxyethyl)[(1-methylpiperid-2-yl)phenylmethyl]amine((S,2S),(R,2R)). (M+H)⁺=293.

### Ex 30b: tert-Butyl 5-{3-(2,2-dimethoxyethyl)-3-[(methylpiperid-2-yl)phenylmethyl]ureido}indazole-1-carboxylate((S,2S),(R,2R))

In a round-bottomed flask equipped with a magnetic bar, 2,2-(dimethoxyethyl)-[(1-methylpiperid-2-yl)phenylmethyl]amine((S,2S),(R,2R)) (0.2 g, 0.86 mmol) is dissolved in 9 mL of DCM at 0° C. Triethylamine (0.16 mL, 1.11 mmol) and triphosgene (0.084 g, 0.28 mmol) are successively added. After stirring for 3 hours at RT, the reaction medium is cooled to 0° C. and tert-butyl 5-amino-3-methylindazole-1-carboxylate (0.376 g, 1.29 mmol) is added. After stirring overnight at RT, the reaction medium is partitioned between 100 mL of DCM and 20 mL of saturated NaHCO₃ solution. After drying the aqueous phase over Na₂SO₄ and evaporation, the residue is purified by chromatography on silica gel (eluent: 95/5/0.5 DCM/MeOH/NH₄OH) to give 0.48 g of tert-butyl 5-{3-(2,2-(dimethoxyethyl)-3-[(1-methylpiperid-2-yl)phenylmethyl]ureido}indazole-1-carboxylate((S,2S),(R,2R)). (M+H)⁺=552.

### Ex 30c 1-(1H-Indazol-5-yl)-3-[(1-methylpiperid-2-yl)phenylmethyl]-1,3-dihydroimidazol-2-one((S,2S),(R,2R))

In a round-bottomed flask equipped with a magnetic bar, tert-butyl 5-{3-(2,2-(dimethoxyethyl)-3-[(1-methylpiperid-2-yl)phenylmethyl]-ureido}indazole-1-carboxylate((S,2S),(R,2R)) (0.36 g, 0.65 mmol) is dissolved in 4 mL of trifluoroacetic acid. After stirring overnight, the solution is neutralized with saturated sodium hydrogen carbonate solution. The medium is extracted with DCM and evaporated. The residue is chromatographed on silica gel (eluent: 93/7 DCM/MeOH) to give 0.18 g of 1-(1H-indazol-5-yl)-3-[(1-methylpiperid-2-yl)phenylmethyl]-1,3-dihydroimidazol-2-one((S,2S),(R,2R)). (M+H)⁺=388, m.p. (fumarate)=231-232° C. ¹H NMR (DMSO, 200 MHz): δ (ppm) 8.04 (d, J=1 Hz, 1H), 7.93 (dd, J=1.0, 1.9 Hz, 1H), 7.66-7.22 (m, 8H), 7.08 (d, J=3.1 Hz, 1H), 6.94 (d, J=3.1 Hz, 1H), 6.57 (s, 2H), 5.29 (d, J=10.8 Hz, 1H), 3.55 (m, 1H), 2.93 (m, 1H), 2.63 (m, 1H), 2.33 (s, 3H), 1.73-1.11 (m, 6H).

### Ex 31: 1-{(S)-(3,4-Dichlorophenyl)[(2S)-piperid-2-yl]methyl}-3-(1H-indazol-5-yl)thiourea

In a three-necked flask equipped with a magnetic stirrer and placed under a nitrogen atmosphere, (S)-1-[(2S)-1-allylpiperid-2-yl]-1-(3,4-dichlorophenyl)methanamine (0.10 g, 0.35 mmol) is dissolved in DCM (2.5 mL). 5-Isothiocyanato-1H-indazole (0.06 g, 0.35 mmol) is added portionwise. The mixture is stirred for 24 hours and concentrated under vacuum. 1-[(S)-[(2S)-1-Allylpiperid-2-yl](3,4-dichlorophenyl)methyl]-3-(−1H-indazol-5-yl)thiourea is obtained. In a three-necked flask equipped with a magnetic stirrer, 1,3-dimethylbarbituric acid (0.16 g, 1.0 mmol) and Pd(PPh₃)₄ (0.04 g, 0.04 mmol) are dissolved in DCM (5 mL) and the mixture is refluxed for 15 minutes. It is then added to the 1-[(S)-[(2S)-1-allylpiperid-2-yl](3,4-dichlorophenyl)methyl]-3-(−1H-indazol-5-yl)thiourea dissolved in DCM (2.5 mL). Two drops of water are added to the reaction medium, which is stirred at reflux for 18 hours. After cooling to RT, the medium is concentrated under vacuum. The residue is purified by chromatography on silica gel diluted with a DCM/MeOH/NH₄OH mixture (99.5/0.5/0.05 to 95/5/0.5). 0.06 g of 1-{(S)-(3,4-dichlorophenyl)[(2S)-piperid-2-yl]methyl}-3-(1H-indazol-5-yl)thiourea is obtained and is treated with fumaric acid in an ethanol/ethyl ether mixture to give the fumarate. (M+H)⁺=434, m.p.=145° C. ¹H NMR (DMSO, 200 MHz): δ (ppm) 8.90 (m, 1H), 8.00 (s, 1H), 7.80 (s, 1H), 7.65-7.20 (m, 5H), 6.55 (s, 2H), 5.45 (d, 1H), 3.25-2.90 (m, 2H), 1.80-1.15 (m, 6H).

### Ex 32: 3-(1H-Indazol-5-yl)-1-methyl-1-[(1-methyl piperid-2-yl)phenylmethyl]urea((S,2S),(R,2R))

### Ex 32a: N-[(1-Methylpiperid-2-yl)phenylmethyl]formamide((S,2S),(R,2R))

In a round-bottomed flask equipped with a magnetic bar, ((S,2S),(R,2R))1-[1-methylpiperid-2-yl]-1-phenylmethylamine (1.57 g, 7.68 mmol) is dissolved in 5 mL of DMF. Formic acid (0.49 mL, 13.06 mmol) is then added and the reaction medium is maintained at 105° C. for 30 minutes. After cooling to RT, the medium is diluted par water, neutralized with NaOH and extracted with a mixture of DCM and isopropanol. After evaporating off the solvents, the residue is taken up in EtOAc and washed with water to give 1.25 g of N-[(1-methylpiperid-2-yl)phenylmethyl]formamide((S,2S),(R,2R)). (M+H)⁺=233.

### Ex 32b: Methyl-[(1-methylpiperid-2-yl)phenylmethyl]amine((S,2S),(R,2R))

In a round-bottomed flask equipped with a magnetic bar, N-[(1-methylpiperid-2-yl)phenylmethyl]formamide((S,2S),(R,2R)) (0.4 g, 1.72 mmol) is dissolved in 15 mL of THF at 0° C. Sodium aluminium hydride (0.327 g, 8.61 mmol) is added and the reaction medium is refluxed for 1 hour. The resulting mixture is allowed to cool to RT and the medium is treated with a solution of NaOH. Extraction with a mixture of DCM and isopropanol gives 0.341 g of methyl-[(1-methylpiperid-2-yl)phenylmethyl]amine((S,2S),(R,2R)). (M+H)⁺=219.

### Ex 32c: tert-Butyl 5-{3-methyl-3-[(1-methylpiperid-2-yl)phenylmethyl]ureido}indazole-1-carboxylate((S,2S),(R,2R))

In a three-necked flask equipped with a magnetic bar, tert-butyl 5-amino-3-methylindazole-1-carboxylate (0.1 g, 0.43 mmol) is dissolved in 6 mL of DCM at 0° C. Triethylamine (0.05 mL, 0.34 mmol) and triphosgene (0.064 g, 0.21 mmol) are successively added. After stirring for 6 hours at RT, methyl-[(1-methylpiperid-2-yl)phenylmethyl]amine((S,2S),(R,2R)) (0.168 g, 0.77 mmol) dissolved in 3 mL of DCM is added. After stirring overnight, the reaction medium is evaporated and the residue is purified by chromatography on silica gel (eluent: 97/3 DCM/MeOH) to give 0.18 g of tert-butyl 3-methyl-5-({[1-methylpiperid-2-yl](phenyl)methyl]carbamoyl}amino)indazole-1-carboxylate((S,2S),(R,2R)). (M+H)⁺=478.

### Ex 32d: 3-(1H-Indazol-5-yl)-1-methyl-1-[(1-methylpiperid-2-yl)phenylmethyl]urea((S,2S),(R,2R))

In a round-bottomed flask equipped with a magnetic bar, tert-butyl 3-methyl-5-({[1-methylpiperid-2-yl](phenyl)methyl]carbamoyl}amino)indazole-1-carboxylate((S,2S),(R,2R)) (0.18 g, 1.88 mmol) is dissolved in 10 mL of MeOH and a 0.5 M methanolic solution of sodium methoxide (5 mL, 2.5 mmol) is added. After stirring for 8 hours, the reaction mixture is poured into water and extracted with a mixture of DCM and isopropanol. After evaporating off the solvents, the residue is purified by chromatography on silica gel (eluent: 93/7 DCM/MeOH) to give 0.03 g of 3-(1H-indazol-5-yl)-1-methyl-1-[(1-methylpiperid-2-yl)phenylmethyl]urea((S,2S),(R,2R)). The product obtained is treated with a molar excess of fumaric acid in ethanol. The fumaric acid salt crystallizes after the addition of diisopropyl ether. (M+H)⁺=378, m.p.=194° C. ¹H NMR (DMSO, 200 MHz): δ (ppm) 8.19 (s, 1H), 7.92 (s, 1H), 7.78 (t, J=1.3 Hz, 1H), 7.45-7.19 (m, 8H), 6.50 (s, 1H), 5.56 (d, J=11.3 Hz, 1H), 3.59-2.73 (m, contains water from the DMSO), 1.73-1.09 (m, 6H).

### Ex 33: 1-(1H-Indazol-5-yl)-1-methyl-3-[(1-methylpiperid-2-yl)(phenyl)methyl]urea((S,2S),(R,2R))

### Ex 33a: 5-Amino-1-{[2-(trimethylsilyl)ethoxy]methyl}indazole

In a Parr flask, 5-nitro-1-{[2-(trimethylsilyl)ethoxy]methyl}indazole (1.8 g, 6.14 mol) is dissolved in 15 mL of methanol and 10% palladium-on-charcoal (0.5 g, 0.25 mmol) is added under N₂. The reaction medium is stirred under 40 psi of hydrogen for 24 hours. After filtering off the catalyst and evaporating off the methanol, 1.5 g of 5-amino-1-{[2-(trimethylsilyl)ethoxy]methyl}indazole are recovered. (M+H)⁺=264.

### Ex 33b: N-[1-{[2-(Trimethylsilyl)ethoxy]methyl}indazol-5-yl]formamide

In a three-necked flask equipped with a magnetic bar, acetic anhydride (1.6 mL, 17.08 mmol) is added to a solution of formic acid (0.64 mL, 17.08 mmol) in 20 mL of THF at 0° C. After stirring for 2 hours, the reaction medium is cooled to −20° C. and 5-amino-1-{[2-(trimethylsilyl)ethoxy]methyl}indazole (1.5 g, 5.69 mmol) dissolved in 5 mL of THF is added. After reaction for 4 hours between −10 and −20° C., the reaction medium is evaporated and the residue is taken up in a solution of NaOH and extracted with DCM. The organic phase is dried over Na₂SO₄ and evaporated to give 1.22 g of N-[1-{[2-(trimethylsilyl)ethoxy]-methyl}indazol-5-yl]formamide. (M+H)⁺=292.

### Ex 33c: Methyl-[1-{[2-(trimethylsilyl)ethoxy]methyl}indazol-5-yl]amine

In a three-necked flask equipped with a magnetic bar, N-[1-{[2-(trimethylsilyl)ethoxy]methyl}indazol-5-yl]formamide (0.515 g, 1.77 mmol) is dissolved in 10 mL of THF and 1 M lithium aluminium hydride in THF (8.9 mL, 8.86 mmol) is added. After stirring for 2 hours, the medium is hydrolysed and extracted with EtOAc to give 0.25 g of methyl-[1-{[2-(trimethylsilyl)ethoxy]methyl}indazol-5-yl]amine. (M+H)⁺=278.

### Ex 33d: 1-Methyl-3-[(1-methylpiperid-2-yl)(phenyl)methyl]-1-(1-{[2-(trimethylsilyl)ethoxy]methyl}-(indazol-5-yl)urea((S,2S),(R,2R))

This compound is prepared according to the process described in Ex. 32c starting with methyl-[1-{[2-(trimethylsilyl)ethoxy]methyl}indazol-5-yl]amine (0.25 g, 0.45 mmol). 0.09 g of 1-methyl-3-[(1-methylpiperid-2-yl)(phenyl)methyl]-1-(1-{[2-(trimethylsilyl)ethoxy]methyl}-(indazol-5-yl)urea((S,2S),(R,2R)) is obtained. (M+H)⁺=508.

### Ex 33e: 1-(1H-Indazol-5-yl)-1-methyl-3-[(1-methylpiperid-2-yl)(phenyl)methyl]urea((S,2S),(R,2R))

This compound is prepared according to the process described in Ex. 17g starting with 1-methyl-3-[(1-methylpiperid-2-yl)(phenyl)methyl]-1-(1-{[2-(trimethylsilyl)ethoxy]methyl}-(indazol-5-yl)urea((S,2S),(R,2R)) (0.043 g, 0.08 mmol). 0.014 g of 1-(1H-indazol-5-yl)-1-methyl-3-[(1-methylpiperid-2-yl)(phenyl)methyl]urea((S,2S),(R,2R)) is obtained. The product obtained is treated with a molar excess of fumaric acid in ethanol. The fumaric acid salt crystallizes after the addition of diisopropyl ether. (M+H)⁺=378, m.p.=205° C. ¹H NMR (DMSO, 200 MHz): δ (ppm) 8.05 (d, J=1 Hz, 1H), 7.65 (d, J=1 Hz, 1H), 7.57 (d, J=8.8 Hz, 1H), 7.34-7.10 (m, 7H), 6.51 (s, 2.5H), 6.09 (d, J=6.8 Hz, 1H), 4.59 (t, J=6.2 Hz, 1H), 3.14 (s, 3H), 2.62 (m, 1H), 2.26 (m, 1H), 2.00 (s, 3H), 1.68-1.06 (m, 6H).

### Ex 34: (3,5-Dichlorophenyl)[piperid-2-yl]methyl 1H-indazol-5-ylcarbamate((S,2S),(R,2R))

### Ex 34a: tert-Butyl 2-[(3,5-dichlorophenyl){[(1-{[2-(trimethylsilyl)ethoxy]methyl}-1H-indazol-5-yl)carbamoyl]oxy}methyl]piperidine-1-carboxylate((S,2S),(R,2R))

In a three-necked flask equipped with a magnetic stirrer and placed under a nitrogen atmosphere, tert-butyl 2-[(3,5-dichlorophenyl)(hydroxy)methyl]piperidine-1-carboxylate (0.46 g, 1.3 mmol) is dissolved in DCM (6 mL) at 4° C. Triethylamine (0.23 mL, 1.7 mmol) and triphosgene (0.11 g, 0.4 mmol) are successively added. The reaction medium is stirred for 6 h at RT. 1-{[2-(Trimethylsilyl)ethoxy]methyl}-1H-indazol-5-amine (0.5 g, 1.9 mmol) dissolved in DCM (3 mL) is then added. The reaction medium is stirred 18 h at RT. It is then washed with saturated sodium hydrogen carbonate solution, dried over Na₂SO₄ and concentrated under vacuum. The residue is purified by chromatography on silica gel eluting with a 7/3 and then 2/1 heptane/EtOAc mixture. 0.4 g of tert-butyl 2-[(3,5-dichlorophenyl){[(1-{[2-(trimethylsilyl)ethoxy]methyl}-1H-indazol-5-yl)carbamoyl]oxy}methyl]piperidine-1-carboxylate is obtained ((S,2S),(R,2R)). (M+H)⁺=649.2

### Ex 34b: (3,5-Dichlorophenyl)[piperid-2-yl]methyl 1H-indazol-5-ylcarbamate((S,2S),(R,2R))

In a round-bottomed flask equipped with a magnetic stirrer and placed under a nitrogen atmosphere, tert-butyl 2-[(3,5-dichlorophenyl){[(1-{[2-(trimethylsilyl)ethoxy]methyl}-1H-indazol-5-yl)carbamoyl]oxy}methyl]piperidine-1-carboxylate((S,2S),(R,2R)) (0.4 g, 0.6 mmol) is dissolved in dioxane (5 mL). A 4N solution of HCl in dioxane (5 mL, 20 mmol) and two drops of water are then added. The reaction mixture is stirred for 5 hours at RT and concentrated under vacuum. The residue is purified by chromatography on silica gel diluted with a DCM/MeOH/NH₄OH mixture (99/1/0.1 to 97/3/0.3). 0.06 g of oil is obtained, which is treated with a 0.2N solution of HCl in ethyl ether. After filtration, (3,5-dichlorophenyl)[piperid-2-yl]methyl 1H-indazol-5-ylcarbamate((S,2S),(R,2R)) is obtained in the form of the hydrochloride. (M+H)⁺=419.2, m.p.=220° C. (hydrochloride) ¹H NMR (DMSO, 200 MHz): δ (ppm) 9.91 (s, 1H), 9.05 (m, 1H), 7.95 (d, J=1 Hz, 1H), 7.82 (d, J=1 Hz, 1H), 7.65 (t, J=1.8 Hz, 1H), 7.51 (d, J=1.8 Hz, 2H), 7.49-7.41 (m, 1H), 7.38-7.30 (m, 1H), 5.67 (d, J=8.5 Hz, 1H), 3.29 (m, 1H), 2.88 (m, 1H), 1.85-1.28 (m, 6H).

### Ex 35: (S)-(3-Chloro-5-fluorophenyl)[(2S)-piperid-2-yl]methyl 1H-indazol-5-ylcarbamate

### Ex 35a: tert-Butyl (2S)-2-[(S)-(3-chloro-5-fluorophenyl){[(1-{[2-(trimethylsilyl)ethoxy]methyl}-1H-indazol-5-yl)carbamoyl]oxy}methyl]piperidine-1-carboxylate

The compound is prepared according to Ex. 34a, replacing the tert-butyl 2-[(3,5-dichlorophenyl)(hydroxy)methyl]piperidine-1-carboxylate with tert-butyl (2S)-2-[(S)(3-chloro-5-fluorophenyl)(hydroxy)methyl]piperidine-1-carboxylate (0.25 g, 0.73 mmol). (M+H)⁺=633.5.

### Ex 35b:(S)-(3-Chloro-5-fluorophenyl)[(2S)-piperid-2-yl]methyl 1H-indazol-5-ylcarbamate

The compound is prepared according to Ex. 34b using the compound prepared in Example 35a.

(M+H)⁺=403.2, m.p.=226° C. (hydrochloride). ¹H NMR (DMSO, 200 MHz): δ (ppm) 9.87 (s, 1H), 9.03 (m, 1H), 7.95 (d, J=1 Hz, 1H), 7.82 (d, J=1 Hz, 1H), 7.53-7.26 (m, 5H), 5.68 (d, J=8.2 Hz, 1H), 3.29 (m, 1H), 2.89 (m, 1H), 1.83-1.30 (m, 6H).

### Ex 36: (3-Chloro-5-fluorophenyl)(1-methylpiperid-2-yl)methyl 1H-indazol-5-ylcarbamate

In a round-bottomed flask equipped with a magnetic stirrer and placed under N₂, the carbamate of Ex. 35 (0.12 g, 0.3 mmol) is dissolved in methanol (2 mL). Formaldehyde (0.11 mL, 1.4 mmol), sodium cyanoborohydride (0.02 g, 0.3 mmol) and acetic acid (0.02 mL, 0.3 mmol) are then successively added. The reaction medium is stirred for 18 hours at RT and then concentrated under vacuum. The residue is purified by chromatography on silica gel eluting with a DCM/MeOH/NH₄OH mixture (98/2/0.2 and then 96/4/0.4). 0.08 g of oil is obtained, which is treated with a 0.2N solution of HCl in ethyl ether. After filtration, (3-chloro-5-fluorophenyl)(1-methylpiperid-2-yl)methyl 1H-indazol-5-ylcarbamate is obtained in the form of the hydrochloride. (M+H)⁺=415.15, m.p.=172° C. (hydrochloride). ¹H NMR (DMSO, 200 MHz): δ (ppm) 12.94 (s, 0.8H), 10.18-9.76 (m, 1.5H), 7.96 (s, 1H), 7.83 (s, 1H), 7.55-7.27 (m, 5H), 6.20-5.73 (m, 1H), 3.93 (m, 1H), 2.83 (s, 3H), 1.98-1.02 (m, 6H).

### Ex 37: (R)-(3,4-Dichlorophenyl)[(2R)-piperid-2-yl]methyl 1H-indazol-5-ylcarbamate

### Ex 37a: tert-Butyl (2R)-2-[(R)-(3,5-dichlorophenyl){[(1-{[2-(trimethylsilyl)ethoxy]methyl}-1H-indazol-5-yl)carbamoyl]oxy}methyl]piperidine-1-carboxylate

The compound is prepared according to Ex. 34a starting with (2R)-2-[(3,4-dichlorophenyl)-(hydroxy)methyl]piperidine-1-carboxylate. (M+H)⁺=649.7

### Ex 37b: (R)-(3,4-Dichlorophenyl)[(2R)-piperid-2-yl]methyl 1H-indazol-5-ylcarbamate

The compound is prepared according to Ex. 34b starting with tert-butyl ((2R)-2-[(R)-(3,5-dichlorophenyl){[(1-{[2-(trimethylsilyl)ethoxy]methyl}-1H-indazol-5-yl)carbamoyl]oxy}methyl]piperidine-1-carboxylate. (M+H)⁺=419.45, m.p.=210° C. (hydrochloride). ¹H NMR (DMSO, 200 MHz): δ (ppm) 9.87 (s, 1H), 9.03 (m, 2H), 7.95 (d, J=1 Hz, 1H), 7.81 (d, J=1 Hz, 1H), 7.71 (m, 2H), 7.48-7.38 (m, 2H), 7.33 (m, 2H), 5.67 (d, J=8.9 Hz, 1H), 3.35-3.23 (m, 1H), 2.89 (m, 1H), 1.82-1.28 (m, 6H).

### Ex 38: (S)-(3,4-Dichlorophenyl)[(2S)-piperid-2-yl]methyl 1H-indazol-5-ylcarbamate and (R)-(3,4-dichlorophenyl)[(2S)-piperid-2-yl]methyl 1H-indazol-5-ylcarbamate

### Ex 38a: tert-Butyl (2S)-2-[(S)-(3,4-dichlorophenyl){[(1-(tert-butoxycarbonyl)-1H-indazol-5-yl)carbamoyl]oxy}methyl]piperidine-1-carboxylate and tert-butyl (2S)-2-[(R)-(3,4-dichlorophenyl){[(1-(tert-butoxycarbonyl)-1H-indazol-5-yl)carbamoyl]oxy}methyl]piperidine-1-carboxylate

Into a three-necked flask equipped with a magnetic stirrer and placed under N₂ is introduced tert-butyl (2S)-2-[(3,4-dichlorophenyl)(hydroxy)methyl]piperidine-1-carboxylate (1.9 g, 5.2 mmol) dissolved in acetonitrile (30 mL). N,N′-Disuccinimyl carbamate (2.05 g, 8 mmol) and triethylamine (2.19 mL, 15.6 mmol) are then added and the reaction medium is stirred for 4 hours at RT. After concentrating the reaction medium by evaporation under RP, the residue thus obtained is taken up in saturated aqueous sodium hydrogen carbonate solution and the aqueous phase is extracted with EtOAc (3×30 mL). The organic phase is washed with aqueous NaCl solution, dried over MgSO₄ and concentrated by evaporation under RP. The residue obtained is diluted in DCM (15 mL). This solution is then added dropwise to solution, prepared beforehand and placed in a one-necked flask, of 5-amino-N-tert-butoxycarbonyl-1H-indazole (1.45 g, 6.2 mmol), DCM (40 mL) and triethylamine (1.1 mL, 7.8 mmol). The reaction medium is stirred at RT overnight. 40 mL of DCM and 30 mL of saturated aqueous sodium hydrogen carbonate solution are then added. After separation of the phases by settling, the organic phase is washed with aqueous NaCl solution, dried over MgSO₄, filtered and concentrated by evaporation under RP. The residue is purified by chromatography on silica gel eluted with a 3/1 cyclohexane/EtOAc mixture. tert-Butyl (2S)-2-[(S)-(3,4-dichlorophenyl){[(1-(tert-butoxycarbonyl)-1H-indazol-5-yl)carbamoyl]oxy}methyl]piperidine-1-carboxylate (0.215 g) is thus obtained the form of a colourless lacquer and tert-butyl (2S)-2-[(R)-(3,4-dichlorophenyl){[(1-(tert-butoxycarbonyl)-1H-indazol-5-yl)carbamoyl]oxy}methyl]piperidine-1-carboxylate (0.434 g) is obtained in the form of a white foam. (M−H)⁻=617.

### Ex 38b: (S)-(3,4-Dichlorophenyl)[(2S)-piperid-2-yl]methyl 1H-indazol-5-ylcarbamate

Into a round-bottomed flask equipped with a magnetic stirrer are introduced tert-butyl (2S)-2-[(S)-(3,4-dichlorophenyl){[(1-(tert-butoxycarbonyl)-1H-indazol-5-yl)carbamoyl]oxy}methyl]piperidine-1-carboxylate (0.269 g) and a 4N solution of HCl in dioxane (4 mL, 15.9 mmol). The reaction mixture is stirred at RT overnight and then concentrated by evaporation under RP. The residue thus obtained is taken up in diisopropyl ether (10 mL) and the resulting solid is filtered through a sinter funnel, washed with diisopropyl ether (2×5 mL) and dried under RP. 0.169 g of (S)-(3,4-dichlorophenyl)[(2S)-piperid-2-yl]methyl 1H-indazol-5-ylcarbamate is thus obtained in the form of the hydrochloride. (M−H)⁺=419, m.p.=194° C. (hydrochloride). [α]_{D}^{20° C.}=+46.5°±1.0 (MeOH) ¹H NMR (DMSO, 400 MHz): δ (ppm) from 1.35 to 1.82 (m, 6H); 2.91 (m, 1H); from 3.25 to 3.75 (partially masked m, 2H); 5.70 (d, J=9.0 Hz, 1H); 7.37 (dd, J=2.0 and 9.0 Hz, 1H); 7.46 (m, 2H); 7.74 (m, 2H); 7.83 (broad s, 1H); 7.99 (s, 1H); from 8.92 to 9.20 (m, 2H); 9.92 (broad s, 1H).

### Ex 38c: (R)-(3,4-Dichlorophenyl)[(2S)-piperid-2-yl]methyl 1H-indazol-5-ylcarbamate

The compound is prepared according to Example 38b starting with (2S)-2-[(R)-(3,4-dichlorophenyl){[(1-(tert-butoxycarbonyl)-1H-indazol-5-yl)carbamoyl]oxy}methyl]piperidine-1-carboxylate. (M−H)⁺=419, m.p.=264° C. (hydrochloride). [α]_{D}^{20° C.}=−4.0°±0.6 (MeOH). ¹H NMR (DMSO, 400 MHz): δ (ppm) from 1.30 to 1.82 (m, 6H); 2.91 (m, 1H); from 3.25 to 3.75 (partially masked m, 2H); 6.06 (d, J=4.0 Hz, 1H); 7.41 (dd, J=2.0 and 8.5 Hz, 2H); 7.50 (d, J=8.5 Hz, 1H); 7.68 (broad s, 1H); 7.73 (d, J=8.5 Hz, 1H); 7.89 (broad s, 1H); 8.00 (s, 1H); 8.80 (m, 1H); 9.20 (m, 1H); 10.05 (broad s, 1H).

### Ex 39: (S)-(3-Chloro-5-fluorophenyl)[(2R)-piperid-2-yl]methyl 1H-indazol-5-ylcarbamate and (R)-(3-chloro-5-fluorophenyl)[(2R)-piperid-2-yl]methyl 1H-indazol-5-ylcarbamate

### Ex 39a: tert-Butyl (2R)-2-[(S)-(3-chloro-5-fluorophenyl){[(1-(tert-butoxycarbonyl)-1H-indazol-5-yl)carbamoyl]oxy}methyl]piperidine-1-carboxylate

The compound is obtained as described in Ex. 38a starting with tert-butyl (2R)-2-[(S)-(3-chloro-5-fluorophenyl)(hydroxy)methyl]piperidine-1-carboxylate (0.6 g, 1.74 mmol). (M−H)⁻=601

### Ex 39b: tert-Butyl (2R)-2-[(R)-(3-chloro-5-fluorophenyl){[(1-(tert-butoxycarbonyl)-1H-indazol-5-yl)carbamoyl]oxy}methyl]piperidine-1-carboxylate

Obtained as described in Ex. 38a starting with tert-butyl (2R)-2-[(R)-(3-chloro-5-fluorophenyl)-(hydroxy)methyl]piperidine-1-carboxylate (0.6 g, 1.74 mmol). (M−H)⁻=601

### Ex 39c: (S)-(3-Chloro-5-fluorophenyl)[(2R)-piperid-2-yl]methyl 1H-indazol-5-ylcarbamate

The compound is obtained as described in Ex. 38b starting with tert-butyl (2R)-2-[(S)-(3-chloro-5-fluorophenyl){[(1-(tert-butoxycarbonyl)-1H-indazol-5-yl)carbamoyl]oxy}methyl]piperidine-1-carboxylate (0.5 g, 0.83 mmol) prepared in Ex. 39a. (M−H)⁺=403, m.p. ˜240° C. (hydrochloride), [α]_{D}^{20° C.}=−8.0°±0.6 (MeOH) ¹H NMR (DMSO, 400 MHz): δ (ppm) from 1.30 to 1.82 (m, 6H); 2.92 (m, 1H); 3.30 (m, 1H); 3.63 (m, 1H); 6.09 (d, J=3.5 Hz, 1H); 7.27 (broad d, J=9.0 Hz, 1H); 7.37 (broad s, 1H); 7.42 (dd, J=2.0 and 8.5 Hz, 1H); 7.48 (partially masked m, 1H); 7.50 (d, J=8.5 Hz, 1H); 7.89 (broad s, 1H); 8.00 (s, 1H); 8.80 (m, 1H); 9.23 (m, 1H); 10.05 (broad s, 1H); 13.0 (broad m, 1H).

### Ex 39d: (R)-(3-Chloro-5-fluorophenyl)[(2R)-piperid-2-yl]methyl 1H-indazol-5-ylcarbamate

The compound is obtained as described in Ex. 38b starting with tert-butyl (2R)-2-[(R)-(3-chloro-5-fluorophenyl){[(1-(tert-butoxycarbonyl)-1H-indazol-5-yl)carbamoyl]oxy}methyl]piperidine-1-carboxylate (0.144 g, 0.238 mmol) prepared in Ex.39b. (M−H)⁺=403, m.p. ˜200° C. (hydrochloride), [α]_{D}^{20° C.}=−57.7°±1.0 (MeOH) ¹H NMR (DMSO, 400 MHz): δ (ppm) from 1.36 to 1.85 (m, 6H); 2.90 (m, 1H); from 3.20 to 3.73 (partially masked m, 2H); 5.72 (d, J=9.0 Hz, 1H); 7.35 (m, 2H); 7.42 (broad s, 1H); 7.50 (m, 2H); 7.84 (broad s, 1H); 8.00 (s, 1H); from 8.80 to 9.10 (m, 2H); 9.86 (broad s, 1H); 12.8 (broad m, 1H).

### Ex 40: (R)-(3-Chloro-5-fluorophenyl)[(2S)-piperid-2-yl]methyl 1H-indazol-5-ylcarbamate

### Ex 40a: tert-Butyl (2S)-2-[(R)-(3-chloro-5-fluorophenyl){[(1-(tert-butoxycarbonyl)-1H-indazol-5-yl)carbamoyl]oxy}methyl]piperidine-1-carboxylate

Obtained as described in Ex. 38a starting with tert-butyl (2S)-2-[(R)-(3-chloro-5-fluorophenyl)-(hydroxy)methyl]piperidine-1-carboxylate (0.2 g, 0.58 mmol). (M−H)⁺=601.

### Ex 40 b: (R)-(3-Chloro-5-fluorophenyl)[(2S)-piperid-2-yl]methyl 1H-indazol-5-ylcarbamate

The compound is obtained as described in Ex. 38b starting with tert-butyl (2S)-2-[(R)-(3-chloro-5-fluorophenyl){[(1-(tert-butoxycarbonyl)-1H-indazol-5-yl)carbamoyl]oxy}methyl]piperidine-1-carboxylate (0.130 g, 0.215 mmol) prepared in Ex. 40a. (M−H)⁺=403, m.p. ˜220° C. (hydrochloride), [α]_{D}^{20° C.}=+14.0° (MeOH). ¹H NMR (DMSO, 400 MHz): δ (ppm) from 1.30 to 1.85 (m, 6H); 2.93 (m, 1H); 3.31 (m, 1H); 3.65 (m, 1H); 6.09 (d, J=4.0 Hz, 1H); 7.27 (broad d, J=9.0 Hz, 1H); 7.35 (broad s, 1H); 7.42 (dd, J=1.5 and 8.5 Hz, 1H); 7.50 (m, 2H); 7.90 (broad s, 1H); 8.00 (s, 1H); 8.71 (m, 1H); 9.15 (m, 1H); 10.05 (broad s, 1H); 13.0 (broad m, 1H).

### Ex 41: Ethyl 3-{[(1H-Indazol-5-ylcarbamoyl)oxy][(2S)-piperid-2-yl]methyl}benzoate

### Ex41a: tert-Butyl 5-[({[(2S)-1-(tert-butoxycarbonyl)piperid-2-yl][3(ethoxycarbonyl)phenyl]methoxy}carbonyl)amino]-1H-indazole-1-carboxylate

Into a one-necked flask equipped with a magnetic stirrer is introduced tert-butyl (2S)-2-{[3-(ethoxycarbonyl)phenyl](hydroxy)methyl}piperidine-1-carboxylate (4.7 g, 12.8 mmol) dissolved in acetonitrile (85 mL) with N,N′-disuccinimidyl carbonate (13.1 g, 51 mmol). Triethylamine (8.95 mL, 64 mmol) is then added and the reaction medium is stirred for 4 hours at a temperature in the region of 20° C. The reaction medium is concentrated to dryness and the evaporation residue is taken up in saturated aqueous sodium hydrogen carbonate solution (50 mL) and extracted with twice 40 mL of EtOAc. A persistent insoluble material is removed by filtration of the organic phases through a sinter funnel. The filtrate is dried over MgSO₄, filtered and concentrated to dryness under RP to give the activated intermediate. Into a second one-necked flask equipped with a magnetic stirrer is introduced tert-butyl 5-amino-indazole-1-carboxylate (3 g, 12.8 mmol) with DCM (125 mL) and triethylamine (2.7 mL, 19.1 mmol). Into this solution is poured the activated intermediate dissolved in DCM (40 mL) over about 10 minutes. The reaction medium is stirred in the region of 20° C. for 16 hours. The medium is hydrolysed with saturated aqueous sodium hydrogen carbonate solution (80 mL), the phases are separated by settling and the aqueous phase is re-extracted with DCM (30 mL). The combined organic extracts are dried over MgSO₄, filtered and concentrated to dryness under RP. The garnet-coloured oil isolated is chromatographed on 420 g of silica gel 60, particle size 15-40 μm, contained in a column 5 cm in diameter, eluting with a 7/3v/v cyclohexane/EtOAc mixture, under an excess pressure of 0.6 bar of argon. The evaporation of the fractions gives 1.53 g of tert-butyl 5-[({[(2S)-1-(tert-butoxycarbonyl)piperid-2-yl][3-(ethoxycarbonyl)phenyl]methoxy}carbonyl)amino]-1H-indazole-1-carboxylate in the form of a white-coloured foam. (M−H)⁻=621. ¹H NMR (DMSO, 400 MHz): 70%-30% mixture of isomers, δ (ppm) from 0.98 to 2.00 (m, 27H); 2.98 (m, 1H); 3.90 (broad m, 1H); 4.33 (q, J=7.5 Hz, 2H); 4.50 (broad m, 1H); 6.09 (d, J=10.0 Hz, 0.7H); 6.23 (broad d, J=9.0 Hz, 0.3H); 7.50 (t, J=7.5 Hz, 0.7H); 7.58 (m, 1.3H); 7.68 (broad d, J=7.5 Hz, 0.7H); 7.75 (broad d, J=7.5 Hz, 0.3H); from 7.85 to 8.00 (m, 3H); 8.04 (broad s, 0.7H); 8.08 (broad s, 0.3H); 8.32 (s, 0.7H); 8.34 (s, 0.3H); 9.82 (broad m, 0.3H); 10.1 (s, 0.7H).

### Ex 41b: Ethyl 3-{[(1H-Indazol-5-ylcarbamoyl)oxy][(2S)-piperid-2-yl]methyl}benzoate

The compound is obtained as described in Ex. 38b starting with tert-butyl 5-[({[(2S)-1-(tert-butoxycarbonyl)piperid-2-yl][3-(ethoxycarbonyl)phenyl]methoxy}carbonyl)amino]-1H-indazole-1-carboxylate prepared in Ex. 41a and stirring for 4 hours instead of overnight. (M−H)⁺=423, m.p. ˜174° C. (hydrochloride). ¹H NMR (DMSO, 400 MHz): δ (ppm) from 1.20 to 1.80 (m, 9H); 2.96 (m, 1H); 3.34 (m, 1H); 3.68 (m, 1H); 4.35 (q, J=7.5 Hz, 2H); 5.74 (d, J=9.0 Hz, 1H); 7.38 (dd, J=2.5 and 8.5 Hz, 1H); 7.47 (d, J=8.5 Hz, 1H); 7.62 (t, J=7.5 Hz, 1H); 7.72 (broad d, J=7.5 Hz, 1H); 7.83 (broad s, 1H); from 7.94 to 8.05 (m, 3H); 9.13 (broad m, 2H); 9.92 (broad s, 1H); 13.1 (broad m, 1H).

### Ex 42: {3-[(4-tert-Butylphenyl)carbamoyl]phenyl}[(2S)-piperid-2-yl]methyl 1H-Indazol-5-yl carbamate

### Ex 42a: tert-Butyl (2S)-2-{[(1H-indazol-5-ylcarbamoyl)oxy][3-(methoxycarbonyl)phenyl]methyl}piperidine-1-carboxylate

Into a one-necked flask equipped with a magnetic stirrer is introduced tert-butyl 5-[({[(2S)-1-(tert-butoxycarbonyl)piperid-2-yl][3-(ethoxycarbonyl)phenyl]methoxy}carbonyl)amino]-1H-indazole-1-carboxylate (1.52 g, 2.4 mmol) prepared in Ex. 41a with lithium hydroxide monohydrate (151 mg, 3.6 mmol), methanol (25 mL) and THF (25 mL). The reaction medium is stirred in the region of 20° C. for 64 hours, after which it is concentrated to dryness under RP. The evaporation residue is taken up in distilled water (30 mL) and the whole is brought to a pH in the region of 5 with 1N HCl (3.6 mL). The solid present is concreted and isolated by filtration, washed with 3×15 mL of distilled water and dried under RP. 1.14 g of tert-butyl (2S)-2-{[(1H-indazol-5-ylcarbamoyl)oxy][3-(methoxycarbonyl)phenyl]methyl}piperidine-1-carboxylate are thus obtained in the form of a white solid. (M+H)⁺=508. ¹H NMR (DMSO, 400 MHz): 70%-30% mixture of isomers, δ (ppm) from 1.05 to 2.00 (m, 15H); 2.98 (m, 1H); 3.86 (s, 2.1H); 3.87 (broad m, 1H); 3.88 (s, 0.9H); 4.48 (broad m, 1H); 6.08 (d, J=10.0 Hz, 0.7H); 6.21 (broad d, J=9.0 Hz, 0.3H); from 7.30 to 8.10 (m, 8H); 9.50 (broad m, 0.3H); 9.82 (broad m, 0.7H); 12.9 (broad m, 1H).

### Ex 42b: 3-{[(2S)-1-(tert-Butoxycarbonyl)piperid-2-yl][(1H-indazol-5-ylcarbamoyl)oxy]methyl}benzoic acid

Into a one-necked flask equipped with a magnetic stirrer is introduced tert-butyl (2S)-2-{[(1H-indazol-5-ylcarbamoyl)oxy][3-(methoxycarbonyl)phenyl]methyl}piperidine-1-carboxylate (1.14 g, 2.24 mmol) with lithium hydroxide monohydrate (293 mg, 6.9 mmol), methanol (5 mL), THF (5 mL), and water (5 mL). The mixture is stirred at about 20° C. for 16 hours, after which it is concentrated to dryness under RP and the residue is taken up in distilled water (30 mL) and then brought to a pH in the region of 4-5 with 1N HCl (6.9 mL). The precipitate formed is then isolated by filtration, washed with distilled water (until the washing waters are neutral), suction-filtered and dried under RP to give 1.14 g of 3-{[(2S)-1-(tert-butoxycarbonyl)piperid-2-yl][(1H-indazol-5-ylcarbamoyl)oxy]methyl}benzoic acid in the form of a white solid. (M+H)⁺=495, m.p. ˜178° C. ¹H NMR (DMSO, 400 MHz): 70%-30% mixture of isomers: from 1.00 to 2.00 (m, 15H); 2.98 (m, 1H); 3.90 (broad m, 1H); 4.47 (broad m, 1H); 6.07 (d, J=10.0 Hz, 0.7H); 6.20 (broad d, J=9.0 Hz, 0.3H); from 7.30 to 7.67 (m, 4H); from 7.80 to 8.10 (m, 4H); 9.52 (broad m, 0.3H); 9.80 (broad m, 0.7H); 12.9 (broad m, 1H).

### Ex 42c: tert-Butyl (2S)-2-({3-[(4-tert-butylphenyl)carbamoyl]phenyl}[(1H-indazol-5-ylcarbamoyl)oxy]methyl)piperidine-1-carboxylate

Into a one-necked flask equipped with a magnetic stirrer are successively introduced 3-{[(2S)-1-(tert-butoxycarbonyl)piperid-2-yl][(1H-indazol-5-ylcarbamoyl)oxy]methyl}benzoic acid ester (580 mg, 1.17 mmol), dimethylformamide (25 mL), 4-tert-butylaniline (187 μL, 1.17 mmol), hydroxybenzotriazole monohydrate (174 mg, 1.28 mmol) and N-methylmorpholine (326 μL, 2.56 mmol). The yellow solution is stirred for 10 minutes at about 20° C., 1-ethyl-3-[3-(dimethylamino)propyl]carbodiimide hydrochloride (247 mg, 1.28 mmol) is then added and the reaction medium is stirred for 16 hours at about 20° C. After concentrating to dryness under RP, the residue is taken up in a mixture of distilled water (30 mL) and EtOAc (20 mL). After separation, the aqueous phase is extracted with twice 15 mL of EtOAc. The combined organic extracts are washed with distilled water (20 mL), saturated aqueous sodium hydrogen carbonate solution (15 mL) and then saturated aqueous NaCl solution (15 mL), dried over MgSO₄, filtered and concentrated to dryness under RP. The isolated residue is chromatographed on 45 g of silica gel 60, particle size 15-40 μm, contained in a column 2 cm in diameter, eluting with a 3/2v/v EtOAc/cyclohexane mixture, under a positive pressure of 0.6 bar of argon. Evaporation of the fractions gives 518 mg of tert-butyl (2S)-2-({3-[(4-tert-butylphenyl)carbamoyl]phenyl}[(1H-indazol-5-ylcarbamoyl)oxy]methyl)piperidine-1-carboxylate in the form of a white foam. (M+H)⁺=626. ¹H NMR (DMSO, 400 MHz): 70%-30% mixture of isomers, δ (ppm) from 1.05 to 2.00 (m, 24H); 2.99 (m, 1H); 3.90 (broad m, 1H); 4.50 (broad m, 0.7H); 4.70 (broad m, 0.3H); 6.10 (d, J=10.0 Hz, 0.7H); 6.24 (broad d, J=9.0 Hz, 0.3H); from 7.30 to 7.70 (m, 4H); 7.38 (d, J=9.0 Hz, 2H); 7.68 (d, J=9.0 Hz, 2H); from 7.80 to 8.10 (m, 4H); 9.58 (broad m, 0.3H); 9.82 (broad m, 0.7H); 10.15 (broad s, 0.7H); 10.25 (broad s, 0.3H); 12.95 (broad m, 1H).

### Ex 42d: {3-[(4-tert-Butylphenyl)carbamoyl]phenyl}[(2S)-piperid-2-yl]methyl 1H-Indazol-5-ylcarbamate

Into a one-necked flask equipped with a magnetic stirrer is introduced tert-butyl (2S)-2-({3-[(4-tert-butylphenyl)carbamoyl]phenyl}[(1H-indazol-5-ylcarbamoyl)oxy]methyl)piperidine-1-carboxylate (500 mg, 0.8 mmol) with 4M hydrochloric acid dissolved in dioxane (7.2 mL, 28.8 mmol). After stirring for 5 hours at about 20° C., the reaction medium is concentrated to dryness under RP and the solid residue is taken up and suspended in isopropyl ether (15 mL). The solid is separated out by filtration, washed with isopropyl ether, suction-filtered and dried under RP. 515 mg of {3-[(4-tert-butylphenyl)carbamoyl]phenyl}[(2S)-piperid-2-yl]methyl 1H-indazol-5-ylcarbamate are thus obtained in the form of a cream-coloured solid. (M+H)⁺=526, m.p. ˜214° C. (dihydrochloride). ¹H NMR (DMSO, 400 MHz): 70%-30% mixture of isomers, δ (ppm) from 1.20 to 1.85 (m, 15H); 2.99 (m, 1H); from 3.25 to 3.80 (partially masked m, 2H); 5.78 (d, J=10.0 Hz, 0.3H); 6.16 (broad s, 0.7H); from 7.35 to 7.68 (m, 4H); 7.37 (d, J=9.0 Hz, 2H); 7.70 (d, J=9.0 Hz, 2H); 7.87 (broad s, 0.3H); 7.91 (broad s, 0.7H); from 7.93 to 8.08 (m, 3H); from 8.63 to 9.25 (m, 2H); 9.91 (broad s, 0.3H); 10.1 (broad s, 0.7H); 10.4 (s, 0.3H); 10.45 (s, 0.7H); 13.0 (broad m, 1H).

### Ex 43: [3-(Anilinocarbonyl)phenyl][(2S)-piperid-2-yl]methyl 1H-Indazol-5-ylcarbamate

### Ex43a: tert-Butyl (2S)-2-{[3-(anilinocarbonyl)phenyl][(1H-indazol-5-ylcarbamoyl)oxy]-methyl}piperidine-1-carboxylate

Into a one-necked flask equipped with a magnetic stirrer are successively introduced 3-{[(2S)-1-(tert-butoxycarbonyl)piperid-2-yl][(1H-indazol-5-ylcarbamoyl)oxy]methyl}benzoic acid (300 mg, 0.6 mmol), dimethylformamide (15 mL), aniline (56 μL, 0.6 mmol), hydroxybenzotriazole monohydrate (89 mg, 0.66 mmol) and N-methylmorpholine (168 μL, 1.32 mmol). The yellow solution is stirred for 10 minutes at about 20° C., 1-ethyl-3-[3-(dimethylamino)propyl]carbodiimide hydrochloride (127 mg, 0.66 mmol) is then added and the reaction medium is stirred for 16 hours at about 20° C. After concentrating to dryness under RP, the residue is taken up in a mixture of distilled water (30 mL) and EtOAc (20 mL). After separation, the aqueous phase is extracted with twice 15 mL of EtOAc. The combined organic extracts are washed with distilled water (20 mL), saturated aqueous sodium hydrogen carbonate solution (15 mL) and then saturated aqueous NaCl solution (15 mL), dried over MgSO₄, filtered and concentrated to dryness under RP. The isolated residue is chromatographed on 30 g of silica gel 60, particle size 15-40 μm, contained in a column 2 cm in diameter, eluting with a 1/1v/v cyclohexane/EtOAc mixture, under a positive pressure of 0.6 bar of argon. Evaporation of the fractions gives 226 mg of tert-butyl (2S)-2-{[3-(anilinocarbonyl)phenyl][(1H-indazol-5-ylcarbamoyl)oxy]methyl}piperidine-1-carboxylate in the form of a colourless lacquer. (M+H)⁺=570. ¹H NMR (DMSO, 400 MHz): 70%-30% mixture of isomers, δ (ppm) from 1.10 to 2.00 (m, 15H); 2.99 (m, 1H); 3.90 (broad m, 1H); 4.50 (broad m, 1H); 6.10 (d, J=10.0 Hz, 0.7H); 6.23 (broad d, J=9.0 Hz, 0.3H); 7.11 (m, 1H); from 7.30 to 8.10 (m, 12H); 9.50 (broad m, 0.3H); 9.80 (broad m, 0.7H); 10.2 (broad s, 0.7H); 10.3 (broad s, 0.3H); 12.9 (broad m, 1H).

### Ex 43b: [3-(Anilinocarbonyl)phenyl][(2S)-piperid-2-yl]methyl 1H-Indazol-5-ylcarbamate

Into a one-necked flask equipped with a magnetic stirrer is introduced tert-butyl (2S)-2-{[3-(anilinocarbonyl)phenyl][(1H-indazol-5-ylcarbamoyl)oxy]methyl}piperidine-1-carboxylate (345 mg, 0.6 mmol) with 4M hydrochloric acid dissolved in dioxane (5.5 mL, 21.8 mmol). After stirring for 5 hours at about 20° C., the mixture is concentrated to dryness under RP and the solid residue is taken up and suspended in isopropyl ether (15 mL). The solid is separated out by filtration, washed with isopropyl ether, suction-filtered and dried under RP. 340 mg of [3-(anilinocarbonyl)phenyl][(2S)-piperid-2-yl]methyl 1H-indazol-5-ylcarbamate are thus obtained in the form of a cream-coloured solid. (M+H)⁺=470, m.p. ˜150° C. (dihydrochloride). ¹H NMR (DMSO, 400 MHz): 70%-30% mixture of isomers, δ (ppm) from 1.20 to 1.85 (m, 6H); 3.00 (m, 1H); from 3.30 to 4.35 (partially masked m, 2H); 5.78 (d, J=10.0 Hz, 0.3H); 6.16 (broad s, 0.7H); 7.11 (t, J=7.5 Hz, 1H); from 7.33 to 7.69 (m, 4H); 7.37 (t, J=7.5 Hz, 2H); 7.80 (d, J=7.5 Hz, 2H); 7.89 (broad s, 0.3H); 7.91 (broad s, 0.7H); from 7.93 to 8.08 (m, 3H); from 8.60 to 9.30 (m, 2H); 9.92 (broad s, 0.3H); 10.1 (broad s, 0.7H); 10.4 (s, 0.3H); 10.45 (s, 0.7H); 13.0 (broad m, 1H).

### Ex 44: (S)-(3-Trifluoromethyl phenyl)[(2S)-piperid-2-yl]methyl 1H-indazol-5-ylcarbamate

### Ex 44a: tert-Butyl (2S)-2-[(S)-(3-trifluoromethyl phenyl){[(1-(tert-butoxycarbonyl)-1H-indazol-5-yl)carbamoyl]oxy}methyl]piperidine-1-carboxylate

The compound is obtained as described in Ex. 38a starting with tert-butyl (2S)-2-[(S)-(3-trifluoromethylphenyl)(hydroxy)methyl]piperidine-1-carboxylate (2 g, 5.56 mmol). (M+H)⁺=619.

### Ex 44 b: (S)-(3-Trifluoromethyl phenyl)[(2S)-piperid-2-yl]methyl 1H-indazol-5-ylcarbamate

The compound is obtained as described in Ex. 38b starting with tert-butyl (2S)-2-[(S)-(3-trifluoromethylphenyl){[(1-(tert-butoxycarbonyl)-1H-indazol-5-yl)carbamoyl]oxy}methyl]piperidine-1-carboxylate (1.2 g, 1.94 mmol) prepared in Ex. 44a. (M+H)⁺=419, m.p. ˜190° C. (hydrochloride). ¹H NMR (DMSO, 400 MHz): δ (ppm) 1.28-1.55 (m, 3H) 1.58-1.81 (m, 3H) 2.94 (m, 1H) 3.34 (m. 1H) 3.61 (partially masked m, 1H) 5.77 (d, J=8.8 Hz, 1H) 7.37 (dd, J=8.8, 2.0 Hz, 1H) 7.47 (d, J=8.8 Hz, 1H) 7.71 (t, J=7.6 Hz, 1H) 7.75-7.86 (m, 4H) 7.98 (s, 1H) 9.01-9.39 (broad m, 2H) 9.99 (broad s, 1H) 12.99 (broad m, 1H).

### Ex 45: (S)-(3-Iodophenyl)[(2S)-piperid-2-yl]methyl 1H-indazol-5-ylcarbamate

### Ex 45a: tert-Butyl (2S)-2-[(S)-(3-Iodophenyl){[(1-(tert-butoxycarbonyl)-1H-indazol-5-yl)carbamoyl]oxy}methyl]piperidine-1-carboxylate

The compound is obtained as described in Ex. 38a starting with tert-butyl (2S)-2-[(S)-(3-iodophenyl)(hydroxy)methyl]piperidine-1-carboxylate (0.85 g, 2.04 mmol). (M+H)⁺=677.

### Ex 45b: (S)-(3-Iodophenyl)[(2S)-piperid-2-yl]methyl 1H-indazol-5-ylcarbamate

The compound is obtained as described in Ex. 38b starting with tert-butyl (2S)-2-[(S)-(3-iodophenyl){[(1-(tert-butoxycarbonyl)-1H-indazol-5-yl)carbamoyl]oxy}methyl]piperidine-1-carboxylate (0.57 g, 0.84 mmol) prepared in Ex. 45a. (M+H)⁺=477, m.p. ˜126° C. (hydrochloride), [α]_{D}^{20° C.}=+54.2°±0.9 (MeOH). ¹H NMR (DMSO, 400 MHz): δ (ppm) 0.97-1.33 (m, 4H) 1.48 (m, 1H) 1.69 (m, 1H) 2.26 (broad m, 1H) 2.47 (partially masked m, 1H) 2.78 (m, 1H) 2.99 (m, 1H) 5.35 (d, J=7.9 Hz, 1H) 7.18 (t, J=7.8 Hz, 1H) 7.34 (dd, J=8.8, 2.0 Hz, 1H) 7.40 (d, J=7.8 Hz, 1H) 7.44 (d, J=8.8 Hz, 1H) 7.67 (d, J=7.8 Hz, 1H) 7.73 (s, 1H) 7.84 (s, 1H) 7.95 (s, 1H) 9.72 (broad s, 1H) 12.91 (broad s, 1H).

### Ex 46: (S)-(3-Bromophenyl[(2S)-piperidyl]methyl 1H-indazolylcarbamate

### Ex 46a: tert-Butyl (S)-2-[(S)-(3-bromophenyl)-(1H-indazol-5-ylcarbamoyloxy)-methyl]piperidine-1-carboxylate

The compound is prepared according to Ex. 38a starting with tert-butyl (S)-2-[(S)-(3-bromophenyl)hydroxymethyl]piperidine-1-carboxylate (0.34 g, 0.92 mmol). (M+H)⁺=629.

### Ex 46b: (S)-(3-Bromophenyl[(2S)-piperidyl]methyl 1H-indazolylcarbamate

The compound is prepared according to Ex. 38b starting with tert-butyl (S)-2-[(S)-(3-bromophenyl)-(1H-indazol-5-ylcarbamoyloxy)methyl]piperidine-1-carboxylate (0.248 g, 0.39 mmol). (M+H)⁺=429, m.p. ˜182° C. (hydrochloride), [α]_{D}^{20° C.}=+77.7°±1.4 (MeOH). ¹H NMR (DMSO, 400 MHz): δ (ppm) 1.29-1.48 (m, 3H) 1.56-1.80 (m, 3H) 2.91 (m, 1H) 3.33 (m. 1H) 3.60 (partially masked m, 1H) 5.68 (d, J=9.2 Hz, 1H) 7.37 (dd, J=8.8, 2.0 Hz, 1H) 7.40-7.50 (m, 3H) 7.63 (dt, J=7.4, 1.5 Hz, 1H) 7.67 (broad s, 1H) 7.85 (broad s, 1H) 7.98 (s, 1H) 8.81-9.12 (broad m, 2H) 9.86 (broad s, 1H) 12.94 (broad m, 1H).

### Ex 47: (S)-(3-Cyclohexylcarbamoylphenyl)-(S)-piperid-2-ylmethyl (1H-Indazol-5-yl)carbamate

### Ex 47a: tert-Butyl (S)-2-[(S)-(3-cyclohexylcarbamoyl phenyl)(1H-indazol-5-ylcarbamoyloxy)-methyl]piperidine-1-carboxylate

The compound is prepared according to Ex. 44a starting with 3-{[(2S)-1-(tert-butoxycarbonyl)piperidyl][(1H-indazol-5-ylcarbamoyl)oxy]methyl}benzoic acid (0.3 g, 0.6 mmol) and cyclohexylamine (70 μL, 0.6 mmol). tert-Butyl (S)-2-[(S)-(3-cyclohexylcarbamoyl-phenyl)-(1H-indazol-5-ylcarbamoyloxy)methyl]piperidine-1-carboxylate (310 mg) is obtained in the form of a white solid. (M+H)⁺=576

### Ex 47b: (S)-(3-Cyclohexylcarbamoylphenyl)-(S)-piperid-2-ylmethyl (1H-Indazol-5-yl)carbamate

The compound is prepared according to Ex. 44b starting with tert-butyl (S)-2-[(S)-(3-cyclohexylcarbamoyl-phenyl)-(1H-indazol-5-ylcarbamoyloxy)methyl]piperidine-1-carboxylate (0.31 g, 0.54 mmol). (S)-(3-Cyclohexylcarbamoylphenyl)-(S)-piperid-2-ylmethyl (1H-indazol-5-yl)carbamate (304 mg) is thus obtained in the form of the hydrochloride. (M+H)⁺=476; ¹H NMR (DMSO, 400 MHz): δ (ppm) 1.13 (td, J=12.0, 8.8 Hz, 1H) 1.22-1.52 (m, 7H) 1.56-1.89 (m, 8H) 2.95 (m, 1H) 3.35 (m, 1H) 3.47-3.83 (partially masked m, 2H) 5.70 (d, J=9.1 Hz, 1H) 7.37 (dd, J=8.8, 2.0 Hz, 1H) 7.46 (d, J=8.8 Hz, 1H) 7.52 (d, J=7.6 Hz, 1H) 7.57 (broad d, J=7.6 Hz, 1H) 7.84 (broad s, 1H) 7.89 (dt, J=7.6, 1.7 Hz, 1H) 7.93 (broad s, 1H) 7.97 (s, 1H) 8.33 (d, J=7.9 Hz, 1H) 9.00-9.18 (broad m, 2H) 9.92 (broad s, 1H) 12.73 (broad m, 1H).

### Ex 48: (7-Fluoro-1H-indazol-5-yl)-(S)-(3,4-dichlorophenyl)-(S)-piperid-2-ylmethyl carbamate

### Ex 48a: 7-Fluoro-5-nitro-1H-indazole

in a three-necked flask equipped with magnetic bar, 7-fluoro-1H-indazole (5.15 g, 37.8 mmol) is dissolved in 50 mL of concentrated sulfuric acid and the mixture is cooled to about 0° C. Potassium nitrate (3.95 g, 39 mmol) is added portionwise. After stirring for 4 hours at 0° C., the mixture is poured onto 600 g of ice. After warming to RT, the precipitate thus formed is filtered and washed with water. The isolated solid is then taken up in aqueous sodium hydrogen carbonate solution and extracted with EtOAc (3×100 mL). The organic phase is washed with water, dried over MgSO₄, filtered and concentrated to dryness. The evaporation residue is purified by chromatography, under a pressure of argon, on silica gel (eluent: 80/20 EtOAc/cyclohexane) to give 1 g of 7-fluoro-5-nitro-1H-indazole. (M+H)⁺=182.

### Ex 48b: 5-Amino-7-fluoro-1H-indazole

7-Fluoro-5-nitro-1H-indazole (1 g, 5.5 mmol) is introduced into a Parr flask in the presence of 400 mg of Pd-on-charcoal (10%) and 80 mL of methanol. The mixture is placed under a 1 bar pressure of H₂ and at 25° C. until the theoretical consumption of the hydrogen. The catalyst is then removed by filtration of the reaction medium through a bed of Clarcel, rinsed with methanol. The filtrate, concentrated to dryness, gives 800 mg of 5-amino-7-fluoro-1H-indazole in the form of a dark red oil, which is used without further purification. (M+H)⁺=152.

### Ex 48c: tert-Butyl 5-amino-7-fluoroindazole-1-carboxylate

The compound is prepared according to Ex. 18b starting with 5-amino-7-fluoro-1H-indazole (800 mg, 5.2 mmol). (M+H)⁺=252.

### Ex 48d: tert-Butyl (S)-2-[(S)-(3,4-dichlorophenyl)(7-fluoro-1H-indazol-5-ylcarbamoyloxy)-methyl]piperidine-1-carboxylate

The compound is prepared according to Ex. 38a starting with tert-butyl 5-amino-7-fluoroindazole-1-carboxylate (0.12 g, 0.48 mmol) and tert-butyl (2S)-2-[(3,4-dichlorophenyl)(hydroxy)methyl]piperidine-1-carboxylate (215 mg, 0.6 mmol). (M+H)⁺=537.

### Ex 48e: (7-Fluoro-1H-indazol-5-yl)-(S)-(3,4-dichlorophenyl)-(S)-piperid-2-ylmethyl carbamate

Into a round-bottomed flask equipped with magnetic stirrer is introduced tert-butyl (S)-2-[(S)-(3,4-dichlorophenyl)(7-fluoro-1H-indazol-5-ylcarbamoyloxy)methyl]piperidine-1-carboxylate (16 mg, 0.03 mmol), with a 1M solution of HCl in ethyl ether (100 μL, 0.11 mmol) and dioxane (1 mL). The mixture is stirred at RT overnight and then concentrated by evaporation under RP. The residue thus isolated is taken up in ethyl ether (3×3 mL), removing the supernatant at each uptake. The solid obtained is then dried in a fume cupboard. (7-Fluoro-1H-indazol-5-yl)-(S)-(3,4-dichlorophenyl)-(S)-piperid-2-ylmethyl carbamate is thus obtained in the form of the hydrochloride. (M+H)⁺=437. ¹H NMR (DMSO, 400 MHz): δ (ppm) 1.37-1.48 (m, 3H) 1.53-1.82 (m, 3H) 2.92 (m, 1H) 3.33 (m, 1H) 3.68 (m, 1H) 5.73 (d, J=9.1 Hz, 1H) 7.31 (dd, J=12.3, 1.0 Hz, 1H) 7.45 (dd, J=8.3, 2.0 Hz, 1H) 7.65 (d, J=1.0 Hz, 1H) 7.72-7.78 (m, 2H) 8.12 (d, J=3.4 Hz, 1H) 8.76-9.04 (broad m, 2H) 9.99 (broad s, 1H) 13.60 (broad m, 1H).

### Ex 49: (7-Fluoro-1H-indazol-5-yl)-(S)-(3-chloro-5-fluorophenyl)-(S)-piperid-2-ylmethyl carbamate

### Ex 49a: tert-Butyl (S)-2-[(S)-(3-chloro-5-fluorophenyl)(7-fluoro-1H-indazol-5-ylcarbamoyloxy)-methyl]piperidine-1-carboxylate

Prepared according to Ex. 38a starting with 5-amino-7-fluoro-1H-indazole prepared in Example 48b (0.45 g, 2 mmol) and tert-butyl (2S)-2-[(3-chloro-5-fluorophenyl)(hydroxy)methyl]piperidine-1-carboxylate (0.89 g, 2 mmol). (M−H)⁻=519

### Ex 49b: (7-Fluoro-1H-indazol-5-yl)-(S)-(3-chloro-5-fluorophenyl)-(S)-piperid-2-ylmethyl carbamate

The compound is prepared according to Ex. 48b starting with tert-butyl (S)-2-[(S)-(3-chloro-5-fluorophenyl)-(7-fluoro-1H-indazol-5-ylcarbamoyloxy)methyl]piperidine-1-carboxylate (0.22 g, 0.42 mmol). (7-Fluoro-1H-indazol-5-yl)-(S)-(3-chloro-5-fluorophenyl)-(S)-piperid-2-ylmethyl carbamate is thus obtained in the form of the hydrochloride. (M+H)⁺=421. ¹H NMR (DMSO, 400 MHz): δ (ppm) 1.38-1.52 (m, 3H) 1.56-1.81 (m, 3H) 2.91 (m, 1H) 3.33 (m. 1H) 3.68 (partially masked m, 1H) 5.72 (d, J=8.3 Hz, 1H) 7.26-7.37 (m, 2H) 7.42 (broad s, 1H) 7.51 (dt, J=8.6, 2.3 Hz, 1H) 7.66 (d, J=1.3 Hz, 1H) 8.12 (d, J=3.4 Hz, 1H) 8.78-9.31 (broad m, 2H) 10.10 (s, 1H) 14.29 (broad m, 1H).

### Ex 50: (6-Fluoro-1H-indazol-5-yl)-(S)-(3-chloro-5-fluorophenyl)-(S)-piperid-2-ylmethyl carbamate

### Ex 50a: tert-Butyl 5-amino-6-fluoro-1H-indazole-1-carboxylate

The compound is prepared according to Ex. 18b starting with 5-amino-7-fluoro-1H-indazole (prepared as described by A. Takami et al., Bioorganic & Medicinal Chemistry 2004, 12(9), 2115-2137). (M+H)⁺=252

### Ex 50 b: tert-Butyl (S)-2-[(S)-(3-chloro-5-fluorophenyl)-(6-fluoro-1H-indazol-5-ylcarbamoyloxy)-methyl]piperidine-1-carboxylate

Prepared according to Ex. 38a starting with tert-butyl 5-amino-6-fluoro-1H-indazole-1-carboxylate (0.73 g, 2.9 mmol) and tert-butyl (2S)-2-[(3-chloro-5-fluorophenyl)(hydroxy)methyl]piperidine-1-carboxylate (1 g, 2.9 mmol). (M+H)⁺=621

### Ex 50c: (6-Fluoro-1H-indazol-5-yl)-(S)-(3-chloro-5-fluorophenyl)-(S)-piperid-2-ylmethyl carbamate

The compound is prepared according to Ex.38b starting with tert-butyl (S)-2-[(S)-(3-chloro-5-fluorophenyl)-(6-fluoro-1H-indazol-5-ylcarbamoyloxy)methyl]piperidine-1-carboxylate (0.58 g, 0.93 mmol). (M+H)⁺=421, m.p. ˜220° C. (hydrochloride). ¹H NMR (DMSO, 400 MHz): δ (ppm) 1.34-1.53 (m, 3H) 1.62 (m, 1H) 1.75 (m, 2H) 2.92 (m, 1H) 3.31 (m, 1H) 3.66 (m, 1H) 5.69 (d, J=8.4 Hz, 1H) 7.35 (dd, J=8.3, 2.2 Hz, 1H) 7.39-7.45 (m, 2H) 7.51 (dt, J=8.4, 2.0 Hz, 1H) 8.00 (d, J=7.5 Hz, 1H) 8.04 (s, 1H) 9.07 (broad m, 2H) 9.35 (s, 1H) 13.00 (broad m, 1H).

### Determination of the IC50 for AKT1

The inhibitory potential of the compounds is evaluated by HTRF, a time-resolved fluorescence technique based on non-radiative energy transfer (FRET). The protein AKT1 used in these studies does not contain the Pleckstrin homology domain (PH domain). It contains an aspartic acid in place of the serine residue in position 473, and amino acids of the hydrophobic domain. The protein AKT1 is phosphorylated with PDK1 on the residue Threonine 308, and amino acids of the kinase domain. This phosphorylation allows the kinase activity of AKT1 to be activated.

Starting with a 10 mM solution of inhibitors, 3 μl are taken and placed in a 96-well polypropylene plate containing 27 μl of 100% DMSO. The various test compounds are diluted in cascade by taking 15 μl of compound, to which are added 30 μl of 100% DMSO. Finally, 3 μl of each of the concentrations are transferred into a new plate containing 97 μl of reaction buffer (Hepes 50 mM, pH 7.5, MgCl₂ 10 mM (reference Prolabo 25.108.238), triton-X100 0.015% (reference USB 22686), glycerol 2.5% (reference Prolabo 24388-295) and DTT 10 mM (reference Sigma ultra D5545) in 3% DMSO.

The various reaction components are added in the following manner:

First, 10 μl of the AKT1 protein solution are placed in a black 96-well polypropylene plate (reference Costar 3694). Next, 10 μl of the inhibitor solution are added. Finally, the reaction is initiated by adding 10 μl of a solution containing both 100 μM of ATP and 10 μM of biotinylated peptide. The sequence of this peptide is as follows: biot-AAAGGGGGRPRAATFAE (reference Neosystem SP000560). The plate is then covered with a plastic film, agitated and finally incubated at RT for 30 minutes. The reaction is finally stopped by adding 50 μl of a mixture of 16.7 nM of streptavidin labelled with allophycocyanin XL665 (reference 611SAXLA from cisbio-international) and 0.998 nM of europium cryptate-coupled anti-phospho-threonine antibody (61PTRKAZ cisbiointernational) in revelation buffer (Hepes-NaOH 100 mM, EDTA 133 mM, KF 400 mM, BSA, 0.1% at pH 7.0). After incubation overnight at 4° C., the plate is read using an Ultra evolution spectrophotometer from Tecan. The machine settings are as follows: excitation at 320 nm, emission at 620 and 665 nm, lag time of 150 μs, integration time of 500 μs. All the tests are performed in duplicate and the mean of the two tests is calculated.

For each point, a delta F is calculated in the following manner:

R(Ratio)=counts_{665 nm}/counts_{620 nm}

ΔR=Rₛₐₘₚₗₑ−R_{blank}

ΔF%=(ΔR/R_{blank})×100

ΔF % is calculated by the Tecan Ultra Evolution machine.

The IC50 values are calculated using equation 205 of the XLFit4 software.

### Determination of the IC50 for S6K1

The inhibitory potential of the compounds for S6K1 is evaluated in an enzymatic test of phosphorylation of substrate in HTRF (Homogenous Time-Resolved Fluorescence) format. Human S6K1 (24-421) T412E protein is expressed in sf21 cells and purified by chromatography on a nickel chelate column. It is activated by PDK1.

The test comprises two steps, both performed at RT. The phosphorylation reaction takes place during the first step and the revelation step is performed during the second. After a preincubation period of 30 minutes between the enzyme and the inhibitor (14.3 μM of inhibitor or a range of concentrations in the reaction medium ranging from 14.3 μM to 0.00024 μM or 42.9 μM to 0.00073 μM (in steps of 3) added in 5 μl in DMSO-ED 30% (vol/vol) and 2.9 nM of enzyme (or kinase buffer for the blank) added in 30 μl in kinase buffer (HEPES/NaOH 50 mM, MgCl₂ 20 mM, DTT 1 mM, glycerol 5%, Tween 20 0.0025%, pH 7.0), the kinase reaction is triggered by addition of 15 μl of a mixture of two substrates (biot-A-A-A-R-A-R-T-S-S-F-A-E-P-G, referred to as GSK3, ref. SP041404E Neosystem, for a final concentration of 0.4 μM, and ATP, prepared in kinase buffer, for a final concentration of 30 μM). The final concentrations of compounds thus obtained during the incubation period range from 10 μM to 0.17 nM and the concentration of enzyme is 2 nM. After incubation for 20 minutes, the reaction is stopped and the revelation initiated by addition of 30 μl of a mixture of streptavidin XL 665 Xlent, ref. 611SAXLB Cis bio international, and a europium cryptate-coupled anti-phospho-GSK3 antibody, ref. 64CUSKAZ Cis bio international, dissolved in the revelation buffer (HEPES/NaOH 166.7 mM, EDTA 221.7 mM, KF 667.7 mM, BSA 0.167%, pH 7.0). The reaction takes place in a black half-well plate ref. 3694 Costar.

A mixture of the reaction medium after each addition of reagent is obtained by agitation for a few minutes at 700-1000 rpm on a Heidolph Titramax 100 machine. The reading is taken on a TECAN Ultra Evolution machine after at least one night and up to 3 nights at 4° C. The reading settings are as follows: excitation wavelength 320 nm, emission wavelengths 620 and 665 nm, lag time 150 μs, integration time 500 μs.

For each point, the ΔF % is calculated as follows:

R(Ratio)=counts_{665 nm}/counts_{620 nm}

ΔR=Rsample−Rblank

ΔF%=(ΔR/Rblank)×100

The activity of the compound is evaluated by means of the percentage of inhibition of the enzymatic activity obtained in the presence of 10 μM of that during the incubation period (14.3 μM for the preincubation). The active compounds (')/0 of inhibition greater than 50% at 10 μM) are next re-evaluated by determining their concentration capable of inhibiting the enzymatic activity by 50% (IC50). The IC50 values are calculated by means of the XLfit4 205 equation.

### Determination of the Antiproliferative Activity

a. Study of the IGF1-Dependent Proliferation of MEF-IGF1 Murine Fibroblast Cells Measured via Incorporation of [14C]-Thymidine

This test is based on the incorporation of [¹⁴C]-thymidine into the DNA of the cells for the S phase of the cell cycle during cell division. The cell line used in this test is the MEF/3T3 Tet-Off Clone 18 line obtained by stable transfection of the human receptor IGF1R in murine MEF/3T3 Tet-Off fibroblasts. To measure the IGF1-dependent cell proliferation, the cells were deprived of serum and cultured for 3 days in the serum-free culture medium in the presence of the growth factor IGF1 which stimulates the proliferation of MEF-IGF1 cells.

Test of Incorporation of [¹⁴C]-Thymidine into MEF-IGF1 Cells

On day 1, the cells were inoculated with 7500 cells per well in Cytostar 96-well microplates (Amersham (GE) RPNQ0162) in 200 μl of EMEM medium (EMEM, Biowhittaker, #BE12-662F) containing 10% foetal calf serum (FCS, TET-BD Biosciences Tet System Approved FBS US-Sourced, #8630-1) and 1% PSG (Penicillin-Streptomycin-Glutamine (PSG), Gibco #10378-016), and incubated at 37° C., 5% CO₂, for 24 hours. On day 2, the cells were washed in the FCS-free EMEM culture medium containing 1% PSG, and incubated in 170 μl of this serum-free culture medium at 37° C., 5% CO₂, for 24 hours. On day 3, the cells were incubated with 10 μl of IGF1 (2 μg/ml of final concentration; recombinant human IGF-1, R & D Systems, #291-G1), 10 μl (0.1 μCi) of [¹⁴C]-Thymidine (NEN NEC-568) plus 10 μl of increasing concentrations of the test molecules, diluted in dimethyl sulfoxide (DMSO, Sigma D2650). The molecules were added in a volume of 10 μl of a 20-fold concentrated solution in a final volume of 200 μl, the final percentage of DMSO being 0.1%. The treated cells were incubated at 37° C., 5% CO₂, for 72 hours. The incorporation of [¹⁴C]-Thymidine was quantified in cpm units (counts per minute) by counting the radioactivity 72 hours after the start of the treatment, using a Micro-Beta radioactivity counter (Perkin-Elmer). The tests were performed in duplicate.

Analysis of the Results

i) the mean±s.e.m. of each series of well replicates was calculated;

ii) the background noise is calculated by preparing cell-free, IGF1-free and untreated control wells; this control was performed in 4 replicates. The background noise was removed at each measurement;

iii) the maximum response is given by positive control wells containing cells in the presence of IGF1 but untreated; this control was performed in 4 replicates;

iv) the minimum response is given by negative control wells containing IGF1-free and untreated cells; this control was performed in 4 replicates;

v) using these maximum (100%) and minimum (0%) response values, respectively, the data were normalized to give a percentage of inhibition of the proliferation induced by IGF1;

vi) a dose-response curve was represented and the IC50 value (concentration of drug that induces 50% inhibition of incorporation of [¹⁴C]-thymidine) was calculated for each molecule. The IC50 values were calculated by linear regression with the XLfit software (IDBS, UK) using formula 205.

b. Study of the Proliferation of Human Tumoral MIA PaCa-2, C-433, LnCaP and MCF7 Cells via Assay of the Intracellular ATP Using the Celltiter-Glo™ Test

The kit CellTiter-Glo™ for measuring cell viability via luminescence (ProMedia) is a homogeneous method for determining the number of viable cells in culture, based on quantification of the ATP present in the cells, and which indicates the presence of metabolically active cells.

The cell lines used in this test were the following: MIA PaCa-2 cells (human pancreatic carcinoma cells, ATCC, CRL-1420), C-433 cells (human Ewing sarcoma cells, DSMZ, ACC 268), LNCaP clone FGC cells (human prostate carcinoma cells, ATCC, CRL-1740), MCF7 cells (human breast carcinoma cells, ECACC, #86012803). The cells MIAPaCa-2 and LNCaP were cultured in D-MEM culture medium (Invitrogen Gibco, #419656-039) containing 10% foetal calf serum (FCS, Invitrogen Gibco, #10500-064) and 2 mM L-Glutamine (Invitrogen Gibco, #25030-024); the C-433 cells were cultured in MCCoy's 5A culture medium (Invitrogen Gibco, #26600-023)/RPMI 1640 (Invitrogen Gibco, #31870-025) (50/50) containing 10% FCS and 2 mM L-glutamine; the MCF7 cells were cultured in EMEM culture medium (EMEM, Biowhittaker, Lonza, #BE12-662F) containing 10% FCS and 2 mM L-Glutamine.

Evaluation of the Cell Viability via Luminescence Measurement: Celltiter-Glo™ Test

On day 1, the cells were inoculated at 1000 (C-433), 2500 (MCF7) and 10000 (LNCaP and MIA PaCa-2) cells per well in black-bottomed 96-well microplates (NUNC fluoronunc, Fisherbioblock 2311K) in 135 μl of whole culture medium and incubated at 37° C., 5% CO₂, for 3 to 6 hours. The cells were then incubated with increasing concentrations of molecules diluted in dimethyl sulfoxide (DMSO, Sigma D2650). The molecules were added in a volume of 15 μl of a 10-fold concentrated solution in a final volume of 150 μl, the final percentage of DMSO being 0.1%. The cells were incubated at 37° C., 5% CO₂, for 96 hours. After 4 days of treatment with the molecules, the CellTiter-Glo™ was performed by following the manufacturer's instructions (Kit Celltiter-Glo Luminescent, PROMEGA #G7571). Briefly, the cell plates were equilibrated for about 30 minutes at RT and 100 μl per well of Celltiter-Glo reagent were added. The cells were incubated for 1 hour at RT for lysis of the cells and stabilization of the signal. The intracellular ATP was quantified by measuring the luminescence in rlu units (relative luminescence units), 96 hours after initiation of the treatment, using a luminescence counter (Wallac). The tests were performed in six replicates.

Analysis of the Results

i) the mean±s.e.m. of each series of well replicates was calculated;

ii) the maximum response is given by positive control wells containing untreated cells; this control was performed in 12 replicates;

iii) the minimum response is given by cell-free and untreated negative control wells; this control was performed in 6 replicates;

iv) using these maximum (100%) and minimum (0%) response values, respectively, the data were normalized to give a percentage relative to the maximum response;

v) a dose-response curve was represented and the IC50 (concentration of drug inducing 50% inhibition of incorporation of [¹⁴C]-thymidine) was calculated for each molecule. The IC50 values were calculated by linear regression with the XLfit software (IDBS, UK) using formula 205.

Table I gives the biochemical activities, i.e. the IC 50 values on AKT1 and S6K1; in Table II, the antiproliferative activities of some of the compounds are given for certain cell lines. It is observed that the test compounds generally have an IC50 value of less than 10000 nM depending on the cell line.

TABLE I
[A: IC50 <100 nM; B: IC50 between 100 and 500 nM and C: IC50 between 500 and 2000 nM]

| Ex. | R₁ | R₂ | R₃ | R₄ | E | stereochemistry | AKT1 | S6K1 |
|---|---|---|---|---|---|---|---|---|
| 1 | H | Me | H | H | 5-NH—CO—NH— | (S, 2S), (R, 2R) | B | |
| 2 | H | Me | H | H | 6-NH—CO—NH— | (S, 2S), (R, 2R) | B | |
| 3 | 3-Cl, 5-F | H | H | H | 5-NH—CO—NH— | (S, 2S) | A | B |
| 4 | 3,4-Cl₂ | H | H | H | 5-NH—CO—NH— | (S, 2S) | A | B |
| 5 | 3-Cl, 5-F | H | H | H | 5-NH—CO—NH— | (S, 2S), (R, 2R), | A | B |
| | | | | | | (S, 2R), (R, 2S) | | |
| 6 | 3,4-Cl₂ | H | H | H | 5-NH—CO—NH— | (R, 2R) | A | B |
| 7 | 3,5-Cl₂ | H | H | H | 5-NH—CO—NH— | (S, 2S) | A | |
| 8 | H | H | H | H | 5-NH—CO—NH— | (S, 2S) | A | C |
| 9 | H | H | H | H | 5-NH—CO—NH— | (R, 2R) | B | C |
| 10 | H | CH₂—CH═CH₂ | H | H | 5-NH—CO—NH— | (S, 2S) | B | |
| 11 | 3-Cl, 5-F | Me | H | H | 5-NH—CO—NH— | (S, 2S), (R, 2R), | A | C |
| | | | | | | (S, 2R), (R, 2S) | | |
| 12 | H | Me | H | 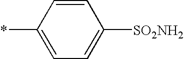 | 5-NH—CO—NH— | (S, 2S), (R, 2R) | A | |
| 13 | H | Me | H | 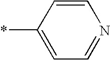 | 5-NH—CO—NH— | (S, 2S), (R, 2R) | B | C |
| 14 | H | Me | H | 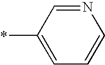 | 5-NH—CO—NH— | (S, 2S), (R, 2R) | A | |
| 15 | H | Me | H | 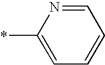 | 5-NH—CO—NH— | (S, 2S), (R, 2R) | B | |
| 16 | 3,4-Cl₂ | H | H | 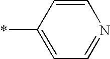 | 5-NH—CO—NH— | (S, 2S) | B | |
| 17 | H | Me | H | 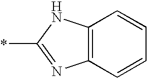 | 5-NH—CO—NH— | (S, 2S), (R, 2R) | A | |
| 18 | H | Me | H | Me | 5-NH—CO—NH— | (S, 2S), (R, 2R) | A | C |
| 19 | H | Me | 7-F | NH—CO—Ph | 5-NH—CO—NH— | (S, 2S), (R, 2R) | C | |
| 20 | H | Me | 7-F | NH₂ | 5-NH—CO—NH— | (S, 2S), (R, 2R) | B | |
| 21 | H | Me | 7-F | H | 5-NH—CO—NH— | (S, 2S), (R, 2R) | B | C |
| 22 | H | Me | H | 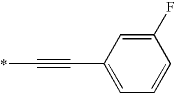 | 5-NH—CO—NH— | (S, 2S), (R, 2R) | B | |
| 23 | H | Me | H | 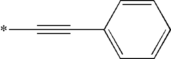 | 5-NH—CO—NH— | (S, 2S), (R, 2R) | A | C |
| 24 | H | Me | H | 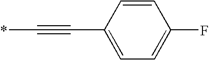 | 5-NH—CO—NH— | (S, 2S), (R, 2R) | A | B |
| 25 | H | Me | H | 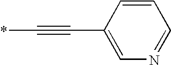 | 5-NH—CO—NH— | (S, 2S), (R, 2R) | A | B |
| 26 | 3,4-Cl₂ | H | H | 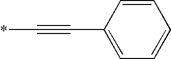 | 5-NH—CO—NH— | (S, 2S) | B | |
| 27 | H | Me | H | CH₂—CH₂—Ph | 5-NH—CO—NH— | (S, 2S), (R, 2R) | B | |
| 28 | H | Me | H | NH—CO—Ph | 5-NH—CO—NH— | (S, 2S), (R, 2R) | B | |
| 29 | 3,4-Cl₂ | H | H | NH₂ | 5-NH—CO—NH— | (S, 2S) | A | C |
| 30 | H | Me | H | H | 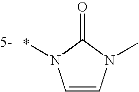 | (S, 2S), (R, 2R) | B | |
| 31 | 3,4-Cl₂ | H | H | H | 5-NH—CS—NH— | (S, 2S) | A | B |
| 32 | H | Me | H | H | 5-NH—CO—N(Me)— | (S, 2S), (R, 2R) | C | |
| 33 | H | Me | H | H | 5-N(Me)—CO—NH— | (S, 2S), (R, 2R) | C | |
| 34 | 3,5-Cl₂ | H | H | H | 5-NH—CO—O— | (S, 2S), (R, 2R) | A | C |
| 35 | 3-Cl, 5-F | H | H | H | 5-NH—CO—O— | (S, 2S) | A | B |
| 36 | 3-Cl, 5-F | Me | H | H | 5-NH—CO—O— | (S, 2S), (R, 2R), | A | C |
| | | | | | | (S, 2R), (R, 2S) | | |
| 37 | 3,4-Cl₂ | H | H | H | 5-NH—CO—O— | (R, 2R) | A | B |
| 38b | 3,4-Cl₂ | H | H | H | 5-NH—CO—O— | (S, 2S) | A | B |
| 38c | 3,4-Cl₂ | H | H | H | 5-NH—CO—O— | (R, 2S) | B | B |
| 39c | 3-Cl, 5-F | H | H | H | 5-NH—CO—O— | (S, 2R) | B | |
| 39d | 3-Cl, 5-F | H | H | H | 5-NH—CO—O— | (R, 2R) | A | |
| 40 | 3-Cl, 5-F | H | H | H | 5-NH—CO—O— | (R, 2S) | C | C |
| 41 | 3-COOEt | H | H | H | 5-NH—CO—O— | (S, 2S), (R, 2S) | C | |
| 42 | 3-CONH-(4-^{t}Bu—Ph) | H | H | H | 5-NH—CO—O— | (S, 2S), (R, 2S) | C | |
| 43 | 3-CONH—Ph | H | H | H | 5-NH—CO—O— | (S, 2S), (R, 2S) | B | |
| 44 | 3-CF₃ | H | H | H | 5-NH—CO—O— | (S, 2S) | A | |
| 45 | 3-I | H | H | H | 5-NH—CO—O— | (S, 2S) | A | |
| 46 | 3-Br | H | H | H | 5-NH—CO—O— | (S, 2S) | A | B |
| 47 | 3-CONH-c-C₆H₁₁ | H | H | H | 5-NH—CO—O— | (S, 2S) | A | B |
| 48 | 3,4-Cl₂ | H | 7-F | H | 5-NH—CO—O— | (S, 2S) | A | |
| 49 | 3-Cl, 5-F | H | 7-F | H | 5-NH—CO—O— | (S, 2S) | A | |
| 50 | 3-Cl, 5-F | H | 6-F | H | 5-NH—CO—O— | (S, 2S) | A | |

TABLE II

| | IC50 [nM] | | | | |
|---|---|---|---|---|---|
| Ex. The | MIA PaCa-2 | MEF-IGF | C-433 | LNCap | MCF7 |
| 3 | 10000 | 790 | 630 | 280 | 1100 |
| 4 | 3420 | 500 | 680 | 1200 | 2700 |
| 5 | 7100 | <100 | | | 1415 |
| 6 | 2900 | | | | |
| 7 | 9320 | 280 | | 5000 | |
| 11 | >10000 | 2922 | | | |
| 13 | 1450 | 5120 | | | |
| 15 | 2800 | | | | |
| 16 | 7790 | | | | |
| 17 | | 2230 | | 670 | |
| 18 | 6100 | 290 | | 1050 | 5800 |
| 20 | 10000 | | | | |
| 21 | | 2820 | | 861 | |
| 22 | 5900 | | | | |
| 23 | 930 | | | | |
| 24 | 1300 | 860 | | | |
| 25 | | 366 | 288 | 611 | 1290 |
| 27 | 1500-7000 | | | | |
| 31 | 6250 | 1270 | | 2940 | 4450 |
| 34 | 2910 | 600 | | | |
| 35 | 3100 | 560 | | 319 | 1050 |
| 36 | 10000 | 770 | | 2232 | |
| 37 | 2450 | 278 | | 2370 | |
| 38b | | 963 | 1786 | 1706 | 2403 |
| 46 | | 546 | 500 | 600 | 1300 |
| 48 | | 295 | 505 | 1350 | 1500 |
| 50 | | 3164 | | | |

What is claimed is:

1. A compound of formula (I): 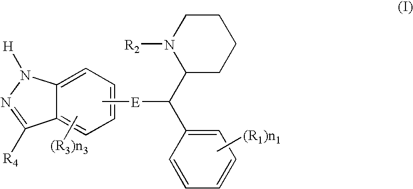
wherein:
E denotes a group:
(i) of formula —NT-CO—O— or —NT-CX-NT′- wherein X denotes ═O or ═S and T and T′, which may be identical or different, are independently chosen from H and an alkyl group or
(ii) forming a 5- or 6-membered ring of formula: 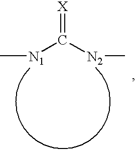
wherein E is attached in position 5 or 6 to the indazole nucleus via —NT- or via the nitrogen atom N₁;
R₁ represents one or more substituents, chosen independently from each other when there are several, from: a halogen atom, an alkyl, alkenyl, alkynyl, haloalkyl, haloalkoxy, aryl, heteroaryl, heterocycloalkyl, cycloalkyl, —CN, —NRR′, —OR, —NO₂, —COOR, —CONRR′, and —NRCOR′;
R₂ represents a hydrogen atom or an alkyl, alkenyl or alkynyl group;
R₃ represents one or more substituents, chosen independently from each other when there are several, from: a halogen atom, an alkyl, alkenyl, alkynyl, haloalkoxy, aryl, heteroaryl, cycloalkyl, heterocycloalkyl, —CN, —NRR′, —CF₃, —OR, —NO₂, —COOR, —CONRR′, and —NRCOR′;
R₄ denotes a hydrogen or halogen atom or an alkyl, alkenyl, alkynyl, aryl, heteroaryl, heterocycloalkyl, —NR—CO—R′, —COOR, —NRR′, —CHO or —CONR(OR′) group;
R and R′, which may be identical or different, denote, independently of each other, a hydrogen atom or an alkyl, aryl, heterocycloalkyl, cycloalkyl or heteroaryl group;
n₁ represents an integer ranging from 0 to 5; and n₃ represents an integer ranging from 0 to 3; or
a pharmaceutically acceptable salt thereof.

2. The compound according to claim 1 wherein E is chosen from —NH—CO—O—, —NH—CO—NH—, —NH—CS—NH—, —NH—CO—N-alkyl- and —N-alkyl-CO—NH—; or
a pharmaceutically acceptable salt thereof.

3. The compound according to claim 1 wherein E is chosen from: 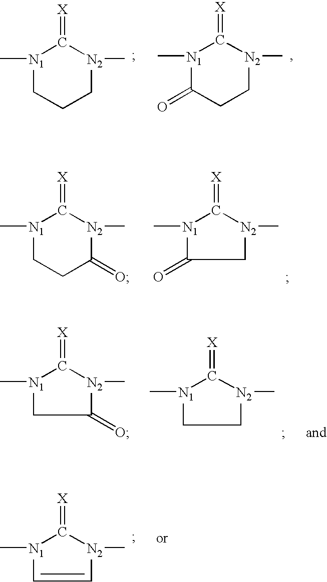
a pharmaceutically acceptable salt thereof.

4. The compound according to claim 1, wherein R₁ denotes a group selected from —CH₂NHR; —C≡C—R; a phenyl group Ph, optionally substituted with at least one substituent chosen from —CH₂OR, —NHCOR, —NHCH₂R; —COOH and —COOalkyl; —CONHPh; phenyl group optionally substituted in position 4 with ^{t}Bu; —CF₃; —OCF₃; and —CONH-c-C₆H₁₁; and wherein R is as defined in claim 1; or a pharmaceutically acceptable salt thereof.

5. The compound according to claim 1 wherein R₂ represents a methyl or allyl group —CH₂—CH═CH₂; or a pharmaceutically acceptable salt thereof.

6. The compound according to claim 1 wherein R₃ denotes a halogen atom or an alkyl group; or a pharmaceutically acceptable salt thereof.

7. The compound according to claim 6 wherein the halogen atom is fluorine and the alkyl group is a methyl group; or a pharmaceutically acceptable salt thereof.

8. The compound according to claim 1 wherein:
R₁ represents one or more substituents, chosen independently from each other when there are several, from a halogen atom, —COOR and —CONRR′;
R₂ represents a hydrogen atom or an alkyl or alkenyl group;
R₃ represents a halogen atom;
R₄ denotes a hydrogen, halogen, alkyl, aryl, heteroaryl, —NR—CO—R′ or —NRR′;
R and R′, which may be identical or different, denote, independently of each other, a hydrogen atom or an alkyl or aryl group;
n₁ represents an integer ranging from 0 to 5; and n₃ represents an integer ranging from 0 to 3; or a pharmaceutically acceptable salt thereof.

9. The compound according to claim 1 wherein R₄ denotes methyl; —CH₂CH₂Ph; a phenyl group, optionally substituted in position 4 with a group —SO₂NH₂; 2-, 3 or 4-pyridyl or benzimidazolyl; —NH—CO-Ph; or —NH₂, or a pharmaceutically acceptable salt thereof.

10. The compound according to claim 1 wherein R₄ denotes a group —C≡C—R wherein R denotes an aryl or heteroaryl group; or a pharmaceutically acceptable salt thereof.

11. The compound according to claim 10 wherein the aryl group is a phenyl group, optionally substituted in position 3 or 4 with a fluorine atom, and the heteroaryl group is 3-pyridyl; or a pharmaceutically acceptable salt thereof.

12. The compound according to claim 1 wherein n₁ represents 0, 1 or 2. and n₃ represents 0 or 1; or a pharmaceutically acceptable salt thereof.

13. The compound according to claim 1 wherein n₁ represents 0; R₂ represents a hydrogen atom or an alkyl or alkenyl group; n₃ represents 0; and R₄ represents a hydrogen atom or an alkyl group; or a pharmaceutically acceptable salt thereof.

14. The compound according to claim 13 wherein R₂ is an alkyl group, and R₄ is a hydrogen atom; or alternatively R₂ and R₄ are an alkyl group; or a pharmaceutically acceptable salt thereof.

15. The compound according to claim 1 wherein R₁ represents a halogen atom; R₂ represents a hydrogen atom or an alkyl group; n₃ represents 0; and R₄ represents a hydrogen atom; or a pharmaceutically acceptable salt thereof.

16. The compound according to claim 1 wherein n₁ represents 2; and R₁ is independently selected from fluorine and chlorine; or a pharmaceutically acceptable salt thereof.

17. The compound according to claim 1 wherein n₁ represents 0; R₂ represents a hydrogen atom or an alkyl group; n₃ represents 0; and R₄ represents an aryl or heteroaryl group; or a pharmaceutically acceptable salt thereof.

18. The compound according to claim 17 wherein R₄ is a phenyl group, optionally substituted with a group —SO₂NH₂ in position 4; or R₄ represents a 2-, 3- or 4-pyridyl group or a benzimidazolyl group; or a pharmaceutically acceptable salt thereof.

19. The compound according to claim 1 wherein n₁ represents 0; R₂ represents a hydrogen atom or an alkyl group; n₃ represents 0; and R₄ represents a group —C═C—R, wherein R denotes an aryl or heteroaryl group; or a pharmaceutically acceptable salt thereof.

20. The compound according to claim 19 wherein R is a phenyl group, optionally substituted with a fluorine atom in position 3 or 4, or R is a 2-pyridyl group; or a pharmaceutically acceptable salt thereof.

21. The compound according to claim 1 wherein n₁=0, R₂ represents a hydrogen atom or an alkyl group, R₃ represents a halogen atom or an alkyl group, and R₄ represents a hydrogen, —NRR′, or —NR—CO—R; or a pharmaceutically acceptable salt thereof.

22. The compound according to claim 21 wherein R₄ represents —NH₂ or —NH—CO-Ph; or a pharmaceutically acceptable salt thereof.

23. The compound according to claim 21 wherein n₃ represents 1; and R₃ is a fluorine atom; or a pharmaceutically acceptable salt thereof.

24. The compound according to claim 1 wherein R₁ is chosen from: an alkyl, alkenyl, alkynyl, haloalkyl, haloalkoxy, heteroaryl, heterocycloalkyl, cycloalkyl, CN, NRR′, OR, NO₂, COOR, CONRR′ or NRCOR′ group, R₂ represents a hydrogen atom or an alkyl group, n₃=0 and R₄ represents a hydrogen atom; or a pharmaceutically acceptable salt thereof.

25. The compound according to claim 24 wherein n₁ represents 1; and R₁ represents 3-COOEt, 3-CONH-(4^{t}Bu)Ph or 3-CONHPh; or a pharmaceutically acceptable salt thereof.

26. The compound according to claim 1 wherein n₁ represents 0, 1 or 2; R₁ represents a halogen atom; R₂ represents a hydrogen atom or an alkyl group; n₃ represents 0 or 1; R₃ represents a halogen atom; and R₄ represents —NH₂, —CH₂CH₂Ph, a hydrogen atom or an alkyl, aryl, heteroaryl or —C≡C—R group; wherein R denotes an aryl or heteroaryl group; or a pharmaceutically acceptable salt thereof.

27. The compound according to claim 26 wherein R₄ represents a phenyl group substituted with —SO₂NH₂ in position 4, or R₄ represents a 2, 3 or 4-pyridyl or benzimidazolyl group; or a pharmaceutically acceptable salt thereof.

28. The compound according to claim 26 wherein R₄ represents —C≡C—R, wherein R denotes a phenyl group, optionally substituted with a fluorine atom in position 3 or 4, or R denotes a 3-pyridyl group; or a pharmaceutically acceptable salt thereof.

29. The compound according to claim 1 wherein E denotes —NH—CO—O— or —NH—CO—NH—, attached via —NH— in position 5 to the indazole; or a pharmaceutically acceptable salt thereof.

30. A compound selected from the group consisting of:
1-(1H-Indazol-5-yl)-3-[(1-methylpiperid-2-yl)(phenyl)methyl]urea((S,2S),(R,2R));
1-(1H-Indazol-6-yl)-3-[(1-methylpiperid-2-yl)(phenyl)methyl]urea((S,2S),(R,2R));
1-{(S)-(3-Chloro-5-fluorophenyl)[(2S)-piperid-2-yl]methyl}-3-(1H-indazol-5-yl)urea;
1-{(S)-(3,4-Dichlorophenyl)[(2S)-piperid-2-yl]methyl}-3-(1H-indazol-5-yl)urea;
1-[(3-Chloro-5-fluorophenyl)(piperid-2-yl)methyl]-3-(1H-indazol-5-yl)urea;
1-{(R)-(3,4-Dichlorophenyl)[(2R)-piperid-2-yl]methyl}-3-(1H-indazol-5-yl)urea;
1-{(S)-(3,5-Dichlorophenyl)[(2S)-piperid-2-yl]methyl}-3-(1H-indazol-5-yl)urea;
1-{(S)-(Phenyl)[(2S)-piperid-2-yl]methyl}-3-(1H-indazol-5-yl)urea;
1-{(R)-(Phenyl)[(2R)-piperid-2-yl]methyl}-3-(1H-indazol-5-yl)urea;
1-{(S)-(Phenyl)[(2S)-1-allylpiperid-2-yl]methyl}-3-(1H-indazol-5-yl)urea;
1-[(3-Chloro-5-fluorophenyl)(1-methylpiperid-2-yl)methyl]-3-(1H-indazol-5-yl)urea;
4-[5-({[(1-Methylpiperid-2-yl)(phenyl)methyl]carbamoyl}amino)-1H-indazol-3-yl]benzenesulfonamide((S,2S),(R,2R));
1-[(1-Methylpiperid-2-yl)(phenyl)methyl]-3-(3-pyrid-4-yl-1H-indazol-5-yl)urea((S,2S),(R,2R));
1-[(1-Methylpiperid-2-yl)(phenyl)methyl]-3-(3-pyrid-3-yl-1H-indazol-5-yl)urea((S,2S),(R,2R));
1-[(1-Methylpiperid-2-yl)(phenyl)methyl]-3-(3-pyrid-2-yl-1H-indazol-5-yl)urea((S,2S),(R,2R));
1-{(S)-(3,4-Dichlorophenyl)[(2S)-piperid-2-yl]methyl}-3-(3-pyrid-4-yl-1H-indazol-5-yl)urea;
1-[3-(1H-Benzimidazol-2-yl)-1H-indazol-5-yl]-3-[(1-methylpiperid-2-yl)(phenyl)methyl]urea((S,2S),(R,2R));
3-Methyl-5-({[(1-methylpiperid-2-yl)(phenyl)methyl]carbamoyl}amino)-1H-indazole((S,2S),(R,2R));
1-(3-Amino-7-fluoro-1H-indazol-5-yl)-3-[(1-methylpiperid-2-yl)(phenyl)methyl]urea((S,2S),(R,2R));
1-(7-Fluoro-1H-indazol-5-yl)-3-[(1-methylpiperid-2-yl)(phenyl)methyl]urea((S,2S),(R,2R));
1-{3-[(3-Fluorophenyl)ethynyl]-1H-indazol-5-yl}-3-[(1-methylpiperid-2-yl)(phenyl)methyl]urea((S,2S),(R,2R));
1-[(1-Methylpiperid-2-yl)(phenyl)methyl]-3-[3-(phenylethynyl)-1H-indazol-5-yl]urea((S,2S),(R,2R));
1-{3-[(4-Fluorophenyl)ethynyl]-1H-indazol-5-yl}-3-[(1-methylpiperid-2-yl)(phenyl)methyl]urea((S,2S),(R,2R));
1-[(1-Methylpiperid-2-yl)(phenyl)methyl]-3-[3-(pyrid-3-ylethynyl)-1H-indazol-5-yl]urea((S,2S),(R,2R));
1-{(S)-(3,4-Dichlorophenyl)[(2S)-piperid-2-yl]methyl}-3-[3-(phenylethynyl)-1H-indazol-5-yl]urea;
1-[(1-Methylpiperid-2-yl)(phenyl)methyl]-3-[3-(2-phenylethyl)-1H-indazol-5-yl]urea((S,2S),(R,2R));
N-[5-({[(1-Methylpiperid-2-yl)(phenyl)methyl]carbamoyl}amino)-1H-indazol-3-yl]benzamide((S,2S),(R,2R));
1-(3-Amino-1H-indazol-5-yl)-3-{(S)-(3,4-dichlorophenyl)[(2S)-piperid-2-yl]methyl}urea;
1-(1H-Indazol-5-yl)-3-[(1-methylpiperid-2-yl)phenylmethyl]-1,3-dihydroimidazol-2-one((S,2S),(R,2R));
1-{(S)-(3,4-dichlorophenyl)[(2S)-piperid-2-yl]methyl}-3-(1H-indazol-5-yl)thiourea;
(3,5-Dichlorophenyl)[piperid-2-yl]methyl 1H-indazol-5-ylcarbamate((S,2S),(R,2R));
(S)-(3-Chloro-5-fluorophenyl)[(2S)-piperid-2-yl]methyl 1H-indazol-5-ylcarbamate;
(3-Chloro-5-fluorophenyl)(1-methylpiperid-2-yl)methyl 1H-indazol-5-yl carbamate;
(R)-(3,4-Dichlorophenyl)[(2R)-piperid-2-yl]methyl 1H-indazol-5-ylcarbamate;
(S)-(3,4-Dichlorophenyl)[(2S)-piperid-2-yl]methyl 1H-indazol-5-ylcarbamate;
(R)-(3,4-dichlorophenyl)[(2S)-piperid-2-yl]methyl 1H-indazol-5-ylcarbamate;
(S)-(3-Chloro-5-fluorophenyl)[(2R)-piperid-2-yl]methyl 1H-indazol-5-ylcarbamate;
(R)-(3-chloro-5-fluorophenyl)[(2R)-piperid-2-yl]methyl 1H-indazol-5-ylcarbamate;
[3-(anilinocarbonyl)phenyl][(2S)-piperid-2-yl]methyl 1H-Indazol-5-ylcarbamate;
(S)-(3-Trifluoromethylphenyl)[(2S)-piperid-2-yl]methyl 1H-indazol-5-ylcarbamate;
(S)-(3-Iodophenyl)[(2S)-piperid-2-yl]methyl 1H-indazol-5-ylcarbamate;
(S)-(3-Bromophenyl[(2S)-piperidyl]methyl 1H-indazolylcarbamate;
(S)-(3-cyclohexylcarbamoyl-phenyl)-(S)-piperid-2-ylmethyl (1H-Indazol-5-yl)carbamate;
(7-Fluoro-1H-indazol-5-yl)-(S)-(3,4-dichlorophenyl)-(S)-piperid-2-ylmethyl carbamate;
(7-Fluoro-1H-indazol-5-yl)-(S)-(3-chloro-5-fluorophenyl)-(S)-piperid-2-ylmethyl carbamate; and
(6-Fluoro-1H-indazol-5-yl)-(S)-(3-chloro-5-fluorophenyl)-(S)-piperid-2-ylmethyl carbamate;
or a pharmaceutically acceptable salt thereof.

31. A pharmaceutical composition comprising at least one compound according to claim 1, or a pharmaceutically acceptable salt thereof, and at least one pharmaceutically acceptable excipient.

32. A pharmaceutical composition comprising at least one compound according to claim 30, or a pharmaceutically acceptable salt thereof, and at least one pharmaceutically acceptable excipient.

33. A method for treating or preventing cancer comprising administering to a patient in need thereof an effective dose of a compound of formula (I) according to claim 1, or a pharmaceutically acceptable salt thereof.

34. A process for preparing a compound of formula (IA): 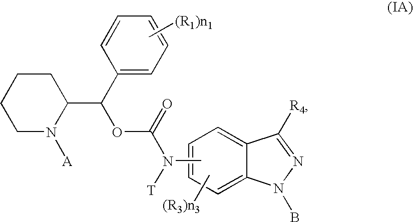
comprising coupling the compounds P₁ and P₂: 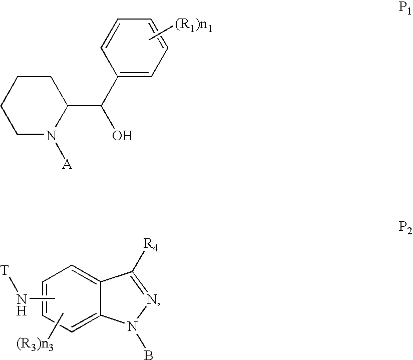
using an agent for introducing the unit C═O;
wherein for the compounds of formula (IA), P₁, and P₂, A represents R₂ as defined in claim 1, except for H, or a protecting group PG₁; B represents H or a protecting group PG₂; and R₁, R₃, R₄, n₁, n₃ and T are as defined in claim 1.

35. A process for preparing a compound of formula (IB): 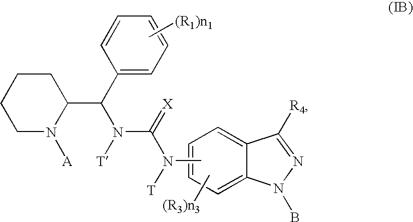
comprising coupling the compounds P₃ and P₂: 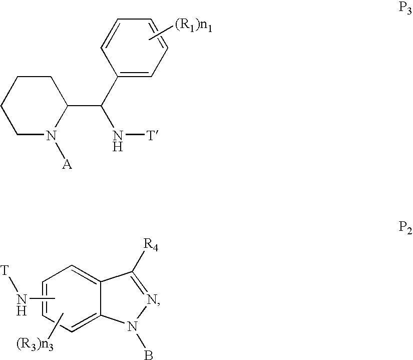
using an agent for introducing the unit C═O or C═S;
wherein for the compounds of formula (IB), P₂, and P₃, A represents R₂ as defined in claim 1, except for H, or A is a protecting group PG₁; B represents H or a protecting group PG₂; and R₁, R₃, R₄, n₁, n₃, X, T and T′ are as defined in claim 1.

36. A process for preparing a compound of formula (IB): 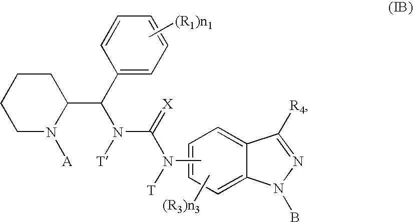
comprising coupling the compounds P₃ and P′₂: 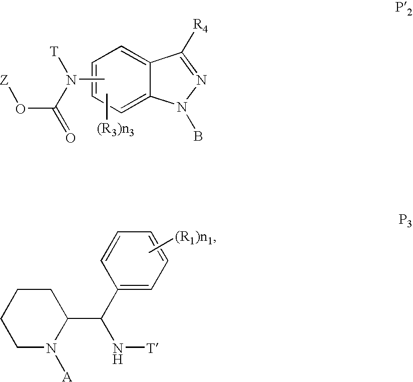
wherein for the compounds of formula (IB), P′₂, and P₃, A represents R₂ as defined in claim 1, except for H, or A is a protecting group PG₁; B represents H or a protecting group PG₂; and R₁, R₃, R₄, n₁, n₃, X, T and T′ are as defined in claim 1, and Z represents a phenyl group, optionally substituted with —NO₂.

37. A process for preparing a compound of formula (IB): 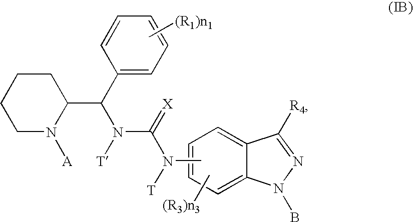
comprising coupling the compounds P₃ and P₄: 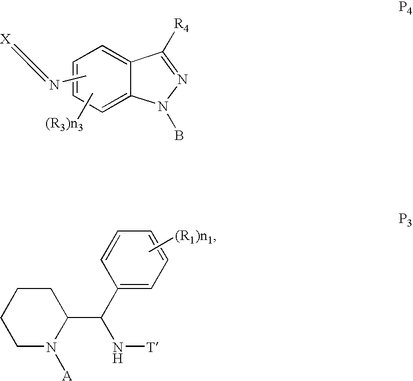
wherein for the compounds of formula (IB), P₄, and P₃, A represents R₂ as defined in claim 1, except for H, or A is a protecting group PG₁; B represents H or a protecting group PG₂; and R₁, R₃, R₄, n₁, n₃, X, T and T′ are as defined in claim 1.

38. A compound of formula (IA): 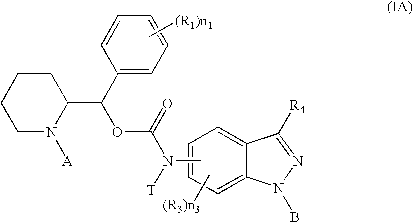
wherein A denotes R₂ as defined in claim 1, except for H, or A is a protecting group PG₁; B denotes H or a protecting group PG₂; and R₁, R₃, R₄, n₁, n₃, T are as defined in claim 1.

39. A compound of formula (IB): 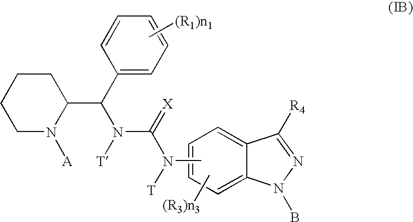
wherein A denotes R₂, except for H, or A is a protecting group PG₁; B denotes H or a protecting group PG₂; and R₁, R₃, R₄, n₁, n₃, X, T and T′ are as defined in claim 1.
